(19) United States
(12) Patent Application Publication
(10) Pub. No.: US 2022/0099659 A1
(43) Pub. Date: Mar. 31, 2022

(54) Human Genomic Construct Reporter Cells and Mouse Models to Screen Therapeutics against Microglia-expressed Disease Associated Genes

(71) Applicant: THE REGENTS OF THE UNIVERSITY OF CALIFORNIA, Oakland, CA (US)

(72) Inventors: Xiangdong William Yang, Los Angeles, CA (US); Xiaofeng Gu, North Hollywood, CA (US)

(21) Appl. No.: 17/425,580

(22) PCT Filed: Jan. 22, 2020
(86) PCT No.: PCT/US2020/014523

§ 371 Date: Jul. 23, 2021

Related U.S. Application Data
(60) Provisional application No. 62/795,657, filed on Jan. 23, 2019.

(51) Int. Cl. G01N 33/50; A01K 67/027

(52) U.S. Cl. CPC G01N 33/5058; A01K 67/0278; A01K 2267/0312; A01K 2217/054; A01K 2227/105; C12N 2800/107

(57) ABSTRACT

The present invention relates to microglial or myeloid expressed Alzheimer's disease associated (ME-AD) gene reporter constructs, cell lines and transgenic animals and their use for identifying agents that modulate the level or expression of ME-AD genes.

### CROSS REFERENCE TO RELATED APPLICATIONS

This application claims priority to U.S. Provisional Application No. 62/795,657, filed Jan. 23, 2019, which is hereby incorporated by reference herein in its entirety.

### BACKGROUND OF THE INVENTION

Microglia are resident innate immune cells in the brain derived from myeloid precursors (Graeber, 2010, Science, 330:783-788). In the healthy brain, resting microglia have ramified processes that constantly survey the microenvironment (Nimmerjahn et al., 2005, Science, 308:1314-1318), and contribute to synaptic plasticity and learning (Parkhurst et al., 2013). In response to injury or neurodegenerative disorders, reactive microglia mediate phagocytic uptake and secretion of inflammatory cytokines (Ransohoff, 2016, Science, 353:777-783). It is generally believed that certain aspects of microglia activation (especially at a short term) may promote tissue repair. However, chronic microglia activation, such as in the case of AD, may elicit neurotoxicity and contribute to disease pathogenesis.

Alzheimer's disease (AD) is the most common cause of dementia worldwide. AD neuropathology is characterized by amyloid plaques, neurofibrillary tangles, neuronal loss, and reactive gliosis and microgliosis. Such pathology affects brain regions critical for memory and cognition including the hippocampus, cerebral cortex, and basal forebrain. The genetic etiology of AD was revealed by studies of rare early-onset familial AD (fAD) patients as well as sporadic late-onset AD patients (LOAD; Karch et al., 2014, Neuron, 83:11-26). The pathogenic fAD-causing mutations in APP, PSEN1 and PSEN2 were found to elevate the generation of pathogenic β-amyloid (Aβ) species and amyloid deposition, hence supporting a pivotal etiological role for amyloid in AD pathogenesis (Hardy and Selkoe, 2002, Science, 297:353-356). By far, the most common and potent genetic risk factor for LOAD is the Apolipoprotein E ε4 allele (APOE4) (Liu et al., 2013, Nat. Rev. Neurol., 9:106-118). Recent genome-wide association studies (GWAS) of LOAD revealed more than 20 AD-associated loci (Karch et al., 2014, Neuron, 83:11-26), implicating multiple innate immunity genes with enriched expression in the microglia as well as peripheral myeloid cells in AD pathogenesis (Efthymiou and Goate, 2017, Mol. Neurodegener, 12:43; Gandy and Heppner, 2013, Neuron, 78:575-577).

Rare variants in the microglia-enriched gene TREM2 confer high risk (2-4.5 fold) for LOAD (Guerreiro et al., 2013, N. Engl. J. Med., 368:117-127; Jonsson et al., 2013, N. Engl. J. Med., 368:107-116; Sims et al., 2017, Nat. Genet., 49:1373-1384). TREM2 is a transmembrane protein selectively expressed in myeloid cells, including microglia (Ulrich et al., 2017, Neuron, 94:237-248; Yeh et al., 2017, Trends Mol. Med., 23:512-533). TREM2 signals through its binding partner DAP12 (TYROBP) to elicit responses including phagocytosis, suppression of proinflammatory response, and promoting cell survival (Poliani et al., 2015, J. Clin. Invest., 125:2161-2170; Takahashi et al., 2005, J. Exp. Med., 201:647-657). The loss-of-function mutations in TREM2 or DAP12 leads to Nasu-Hakola disease, a recessive disorder characterized by bone cysts and early dementia (Paloneva et al., 2002, Am. J. Hum. Genet., 71:656-662), highlighting a key role of TREM2 in microglia and related myeloid cells in age-dependent disease processes. Importantly, a subset of the loss-of-function TREM2 variants are found to predispose to a frontotemporal dementia-like syndrome without apparent bone involvement (Guerreiro et al., 2013, AMA Neurol., 70:78-84). Together, human genetics suggest that an in-depth understanding of TREM2 biology in microglia could provide insights into the pathogenesis of AD and related neurodegenerative disorders.

Recent studies have begun to unravel the functional roles of TREM2 in molecular, cellular and animal models that are informative to AD. Substantial effort has been devoted to identify TREM2 ligands. TREM2 has been shown to bind anionic and zwitterionic lipids found on damaged neurons (Wang et al., 2015, Cell, 160:1061-1071) and AD-associated proteins APOE and Clusterin (Atagi et al., 2015, J. Biol. Chem., 290:26043-26050; Bailey et al., 2015, J. Biol. Chem., 290:26033-26042; Yeh et al., 2016, Neuron, 91:328-340). In APP mouse models, Trem2 plays a role in clustering and activating microglia around Aβ plaques (Jay et al., 2015, J. Exp. Med., 212:287-295; Wang et al., 2015, Cell, 160:1061-1071). The impact of Trem2 deficiency on amyloid plaque formation is dynamic and complex. At an early disease stage, the plaque load is reduced in an AD model crossed to Trem2 knockout mouse (Jay et al., 2015, J. Exp. Med., 212:287-295), while in more advanced disease stages, the plaque load increased in multiple AD models (Jay et al., 2017, J. Neurosci., 37:637-647; Wang et al., 2015, Cell, 160:1061-1071). Moreover, Trem2 in plaque-associated microglia plays an important role in forming a barrier to surround and insulate the plaques, leading to plaque compaction and preventing the spread of neurotoxic fibrillary Aβ (Wang et al., 2016, J. Exp. Med., 213:667-675; Yuan et al., 2016, Neuron, 90:724-739; Condello et al., 2018, Biol Psychiatry, 83:377-387). Thus far, most Trem2 studies in disease models in vivo have used loss-of-function mutants, yet very little is known about the impact of increased Trem2 expression under its genomic regulation on normal brain function and in disease responses.

Recent studies showed TREM2 played a critical role in age-dependent microglial proliferation and survival in the mammalian brain (Poliani et al., 2015, J. Clin. Invest., 125:2161-2170; Krasemann et al., 2017, Immunity, 47:566-581; Ulland et al., 2015, Neuron, 94:237-248). Moreover, recent transcriptomic studies of microglia/myeloid cells at population (Wang et al., 2015, Cell, 160:1061-1071) or single-cell levels (Keren-Shaul et al, 2017, Cell, 169:1276-1290) revealed that TREM2 is essential in the activation of microglia in a variety of disease models, including but not limited to models of amyotrophic lateral sclerosis (ALS), multiple sclerosis (MS), and Alzheimer's disease (AD). These activated microglia glia express a transcriptomic signature that has been called, among other names, the disease-associated microglia (DAM; Deczkowska et al., 2018, Cell, 173:1073-1081). Moreover, the impact of TREM2 in brain disorders may not be limited to its role in microglia, as its expression in peripheral myeloid cells that could enter under certain disease conditions may also play a modifying role in these diseases (e.g. Jay et al., 2015, J. Exp. Med., 212:287-295). Finally, GWAS studies suggest certain pathogenic variants of TREM2 (e.g. R47H) is significantly associated with increased risk for other neurodegenerative disorders beyond AD, which include ALS, Parkinson's disease (PD), FTD, etc (Lill et al., 2015, Alzheimers Dement. 11:1407-1416). These results suggest that proper TREM2 function may be critical for age-dependent microglial survival and proper function of microglia and myeloid cells in the context of age-dependent brain disorders.

Thus, there remains a need in the art for mammalian animal and cell models to enable the identification of molecules that target microglia and myeloid cell function. The present invention fulfills these needs.

### SUMMARY OF THE INVENTION

In one embodiment, the invention relates to a composition comprising a microglial or myeloid expressed Alzheimer's disease associated (ME-AD) gene reporter construct.

In one embodiment, the construct comprises a genomic regulatory element of a ME-AD gene operably linked to at least one sequence encoding a reporter molecule. In one embodiment, the genomic regulatory element is a promoter, a transcriptional enhancer, a transcriptional repressor, a locus control region, a splicing regulatory element, a mRNA polyadenylation site, a trafficking element or a stability regulatory element.

In one embodiment, the construct comprises a ME-AD gene operably linked to at least one sequence encoding a reporter molecule. In one embodiment, the ME-AD gene is selected from the group consisting of TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN and MEF2C. In one embodiment, the ME-AD gene is TREM2.

In one embodiment, the reporter molecule is luciferase.

In one embodiment, the reporter construct is a bacterial artificial chromosome (BAC).

In one embodiment, the reporter construct is integrated into the genome of a cell.

In one embodiment, the invention relates to a cell comprising a ME-AD reporter construct.

In one embodiment, the invention relates to a germline-transmitted genome engineered animal (e.g. transgenic animal) comprising a ME-AD reporter construct.

In one embodiment, the invention relates to a method of screening for a modulator of a ME-AD gene, the method comprising: a) contacting a cell comprising at least one ME-AD reporter construct with an agent, b) measuring the expression level of at least one reporter molecule, and c) comparing the expression level of at least one reporter molecule to the level of a comparator control.

In one embodiment, the ME-AD reporter construct comprises a genomic regulatory element of a ME-AD gene operably linked to at least one sequence encoding a reporter molecule. In one embodiment, the genomic regulatory element is a promoter, a transcriptional enhancer, a transcriptional repressor, a locus control region, a splicing regulatory element, a mRNA polyadenylation site, a trafficking element or a stability regulatory element.

In one embodiment, the ME-AD reporter construct comprises a ME-AD gene operably linked to at least one sequence encoding a reporter molecule.

In one embodiment, the ME-AD gene is TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN or MEF2C. In one embodiment, the ME-AD gene is TREM2.

In one embodiment, the reporter molecule is luciferase.

In one embodiment, the reporter construct is a bacterial artificial chromosome (BAC).

In one embodiment, the reporter construct is integrated into the genome of a cell.

In one embodiment, the human genomic construct is integrated into the genome of a rodent cell and replacing the homologous genomic segment in the rodent genome.

In one embodiment, the comparator control is a positive control, a negative control, a historical control, a historical norm, or the level of a reference molecule in the biological sample.

In one embodiment, the agent is selected from the group consisting of a small interfering RNA (siRNA), a small guide RNA (gRNA), a microRNA, an antisense or sense nucleic acid, a ribozyme, an expression vector encoding a transdominant negative mutant, an antibody, a peptide, a chemical compound and a small molecule.

### BRIEF DESCRIPTION OF THE DRAWINGS

For the purposes of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1L, depicts the generation and characterization of BAC-TREM2 mice. FIG. 1A depicts a schematic representation of the modification of TREM2-BAC. Red crosses indicate the deleted exons in TREM-like genes in the BAC construct. FIG. 1B depicts a UCSC genome browser track showing read coverage at the human TREM2 locus in TREM2 transgenic and WT animals. FIGS. 1C-1K depict images of brain sections from 1.5- to 2-month-old BAC-TREM2-GFP mice, which were double stained with GFP and cell-specific markers for microglia (Iba+, FIG. 1C-1E), astrocytes (GFAP+, FIG. 1F-1H), or neurons (NeuN+, FIG. 1I-1K). Representative cortical images showed that BAC-TREM2-GFP colocalized with Iba1 (FIG. 1C-1E) but not with GFAP (FIG. 1F-1H) or NeuN (FIG. 1I-1K). Bar, 100 μm. FIG. 1L depicts the percentage of double-labeled cells having colocalization of GFP with cell-specific markers. The numbers below x axis indicates the number of cells counted.

FIG. 2, comprising FIG. 2A through FIG. 2D, depicts exemplary results demonstrating that the human- and mouse-specific reads for TREM2 and APP show the specificity of BAC-TREM2 expression and its effect on the expression of endogenous Trem2 and APP. FIGS. 2A and 2B depict the human and mouse TREM2 specific reads, respectively, from 2, 4, and 7 months old mice. n=6 per genotype. Statistical comparison within each age group was performed using one-way ANOVA with Tukey post-hoc analysis. **p<0.01, *p<0.05. FIGS. 2C and 2D depict the human and mouse APP specific reads, respectively, from 2, 4, and 7 months old mice. n=6 per genotype.

FIG. 3, comprising FIG. 3A through FIG. 3D, depicts exemplary results demonstrating that BAC-TREM2 mice showed specific microglial expression of the transgene and normal electrophysiological and behavioral properties. FIGS. 3A through 3C depict brain sections from 1.5-2 months old BAC-TREM2-GFP mice which were double stained for GFP and Iba1. Representative hippocampal images showing BAC-TREM2-GFP labeling in Iba1+ microglia. FIG. 3D depicts exemplary experimental results demonstrating that open-field exploratory behavior was recorded at 2 months of age. Total distance of ambulatory movement during 15 minutes of test are presented as mean±SEM. n=6 per genotype.

FIG. 4, comprising FIG. 4A through FIG. 4J, depicts exemplary results demonstrating that increased TREM2 gene dosage ameliorates amyloid pathology and remodels amyloid plaque types. FIGS. 4A through 4C depict matched brain sections from 7-month-old 5×FAD (FIG. 4A) and 5×FAD/TREM2 mice (FIG. 4B) were stained with ThioS and NeuN to visualize the amyloid plaques and neurons in the cortex, respectively. Z stack confocal images (20×) were utilized to measure total plaque area in the field using ImageJ. The results are presented as ThioS+ plaque area (μm2) per mm2 of the cortical area (FIG. 4C). n=7 per genotype, **p<0.01. Bar, 50 μm. FIGS. 4D through 4G depict the levels of soluble and insoluble Aβ42 (FIG. 4D and FIG. 4E) and Aβ40 (FIG. 4F and FIG. 4G) in the cortex of 4- and 7-month-old mice were measured by ELISA. n=6 per genotype, *p<0.05. FIGS. 4H through 4J depict matched brain sections from 7-month-old 5×FAD (FIG. 4H) and 5×FAD/TREM2 mice (FIG. 4I) were stained with ThioS and an anti-Aβ antibody (6E10). z stack confocal images (40×) were utilized to quantify 3 different forms of plaques using ImageJ (FIG. 4J). A total of 502 plaques were analyzed and are presented as mean±SEM, n=4 per genotype, **p<0.01, ***p<0.001. Bar, 50 μm.

FIG. 5, comprising FIG. 5A through FIG. 5B, depicts exemplary results demonstrating that increased TREM2 gene dosage reduces 6E10+ plaque burden. Matched brain section from 7 months old 5×FAD (FIG. 5A) and 5×FAD/TREM2 mice (FIG. 5B) were stained with ThioS and 6E10 to visualize the amyloid plaques in the cortex. Z-stack confocal images (20×) were utilized to measure total plaque area in the field using ImageJ. The results are presented as number of 6E10+ plaques per mm2 of the cortical area. n=7 per genotype, *p<0.05.

FIG. 6, comprising FIG. 6A through FIG. 6I, depicts the results of a principal component analysis and the numbers of differentially expressed genes for various genotype contrasts and genotype-gender interaction tests of RNA-seq data. FIG. 6A through FIG. 6C depict that the first two principal components of the data of 2, 4, and 7 months old mice were presented. Each dot represents the result from one mouse. FIGS. 6D through 6I depict the numbers of significantly (FDR<0.1) down- and upregulated genes, respectively. FIG. 6D through FIG. 6F indicate numbers of significantly differentially expressed genes in genotype comparisons in 2, 4, and 7 months old samples as indicated. FIG. 6G through FIG. 6I show the numbers of genes with significant interaction of genotype and gender, i.e. genes whose differential expression between genotypes is significantly different in male vs. female samples. This includes genes that show significant differential expression (DE) only in one of the genders or where the fold change in male samples has the opposite sign (direction) than in female samples. Blue/red bars represent significantly down-/up-regulated genes. For each genotype, n=6 samples (3 male and 3 female), except n=5 samples (2 female and 3 male) for 7-month 5×FAD.

FIG. 7, comprising FIG. 7A through FIG. 7E, depicts that a transcriptomic and coexpression network analyses reveal partial rescue of transcriptomic changes in 5×FAD mice with increased TREM2 gene dosage. FIG. 7A depicts the numbers of DE genes (FDR<0.1) in genotype contrasts. Bars represent significantly down-/upregulated genes. n=6 per genotype and time point except n=5 for 5×FAD at 7 months. FIG. 7B and FIG. 7C depict the transcriptome-wide rescuing effects of increased TREM2 gene dosage in mice 4 (FIG. 7B) and 7 (FIG. 7C) months old are presented as “rescue plots.” The plots show Z statistics for DE in 5×FAD versus 5×FAD/TREM2 (y axis) and 5×FAD versus WT (x axis) for all genes (each gene corresponds to one point). Rescued (concordant in this plot) and exacerbated (discordant) genes that pass the FDR threshold of 0.1 in both comparisons are shown in blue and red, respectively. Genome-wide correlations of Z statistics and the corresponding correlation p values (for which the n=15,809 genes are considered independent) are indicated on the top of each panel. FIG. 7D depicts the DE analysis of module eigengenes. Rows correspond to modules and columns to selected genotype contrasts. Numbers in the heatmap show the Z statistics and the corresponding p values of module eigengene association with the genotype. n=6 per genotype and time point except n=5 for 5×FAD at 7 months. FIG. 7E depicts the variation of module eigengene expression with age in WT, 5×FAD, and 5×FAD/TREM2 samples. Points represent means of eigengene values across samples at the same age. Error bars, SEM. Network of top 10 hub genes are presented on the right.

FIG. 8, comprising FIG. 8A through FIG. 8D, depicts scatterplots of differential expression statistics between male and female samples show significant concordant expression pattern between genders. FIG. 8A depicts a scatterplot of differential expression statistics between 5×FAD vs. WT at 4 months. FIG. 8B depicts a scatterplot of differential expression statistics between 5×FAD vs. WT at 7 months. FIG. 8C depicts a scatterplot of differential expression statistics between 5×FAD/TREM2 vs. 5×FAD at 4 months. FIG. 8D depicts a scatterplot of differential expression statistics between 5×FAD/TREM2 vs. 5×FAD at 7 months. Each panel shows differential expression significance Z statistics for one genotype contrast in female samples vs. male samples. Each dot represents a gene. The correlation of the Z statistics and the corresponding Student significance p value are shown on the top of each plot. For the significance calculation, each gene is considered independent (n=15809).

FIG. 9 depicts a differential expression analysis of module eigengenes. Rows correspond to modules and columns to selected genotype comparisons. Numbers in the heatmap show the Z statistic and the corresponding Student p-values of module eigengene association with the genotype. There are n=6 samples for each genotype/age except 5×FAD at 7-month, for which there are only 5 samples.

FIG. 10, comprising FIG. 10A through FIG. 10F, depicts increased TREM2 gene dosage reprogrammed disease-associated microglia gene expression. FIG. 10A depicts a heatmap representation of differential expression Z statistics for TD1-3 genes. Genes are divided into TD1, TD2, and TD3 (TD stands for TREM2 Dosage dependent). FIG. 10B and FIG. 10C depict fold changes of individual TD1 (FIG. 10B) and TD2 (FIG. 10C) genes in 5×FAD and 5×FAD/TREM2 versus WT mice at 7 months. Stars indicate FDR-corrected significance (***FDR<0.001, **FDR<0.01, *FDR<0.1). n=6 per genotype, except n=5 for 5×FAD. FIG. 10D through FIG. 10F depict Venn diagrams of overlaps of TD1 and TD2 genes with published gene sets.

FIG. 11 depicts real-time PCR analyses to validate transcripts of selected TD genes. Total RNA isolated from cortical tissues of 7 months old mice were reverse-transcribed to cDNA and followed by real-time PCR quantification with primers specific to selected TD1 (Ccl6, Tyrobp, Siglech, Ch25h, Mpeg1 and Gusb), TD2 (Lgals3, Atp6v0d2 and Spp1) and TD3 (Erbb2 and Zfp536) genes. Gapdh was used for loading control. The expression of transcripts was first normalized to internal Gapdh levels and then compared with WT. The levels of transcripts were presented as fold change over the WT controls. One-way ANOVA with Tukey post-hoc analysis was performed. The statistics are presented as comparing to WT controls unless specifically indicated in the graph. ***p<0.001, **p<0.01, *p<0.05.

FIG. 12, comprising FIG. 12A through FIG. 12H, depicts upregulation of TREM2 altered microglial response to the amyloid plaque. FIG. 12A through FIG. 12H depict representative images demonstrated the interaction between microglia (Iba1⁺) and the plaque (6E10⁺) in 7-month-old 5×FAD (FIG. 12A through FIG. 12D) and 5×FAD/TREM2 mice (FIG. 12E through FIG. 12H). Bar, 25 μm. The images showed the dramatic upregulation of Iba1 expression and formation of activated, ameboid morphology of plaque associated microglia in the 5×FAD cortices (FIG. 12A through FIG. 12D). However, in the 5×FAD/TREM2 cortices, the Iba1 expression level is only moderately elevated and the morphology is more ramified in the amyloid plaque associated microglia (FIG. 12E through FIG. 12H).

FIG. 13, comprising FIG. 13A through FIG. 13G, depicts increased TREM2 gene dosage upregulated expression of phagocytic markers and enhanced phagocytic activity in microglia. FIG. 13A through FIG. 13C depict matching cortical sections from 7-month-old 5×FAD (FIG. 13A) and 5×FAD/BAC-TREM2 mice (FIG. 13B) were stained with CD68, Iba1, and ThioS. FIG. 13C depicts the area of CD68⁺ labeling per plaque was measured on z stack confocal images. n=4 per genotypes, *p<0.05. Bar, 25 μm. FIG. 13D through FIG. 13F depict matching cortical sections from 7-month-old 5×FAD (FIG. 13D) and 5×FAD/BAC-TREM2 mice (FIG. 13E) stained with Lgals3 and Congo red. FIG. 13F depicts the number of Lgal3⁺cells per plaque were counted under a microscope by a blinded observer. n=3 per genotypes, ***p<0.001. Bar, 50 μm. FIG. 13G depicts and analysis of phagocytosis of Alexa-488-conjugated microspheres by primary microglia as measured by flow cytometry. Phagocytic microglia were detected with strong fluorescent signal in the cells. The graph shows pooled results from 4 independent experiments and presented as mean±SEM, n=3-6 per genotypes; **p<0.01, *p<0.05 compared to WT; ^{#}p<0.05 compared to Trem2^{+/-}; ^{§}p<0.05 compared to Trem2^{-/-}.

FIG. 14, comprising FIG. 14A through FIG. 14B, depicts increased TREM2 gene dosage upregulated expression of CD68 around the plaques. Matching cortical sections from 7 months old 5×FAD (FIG. 14A) and 5×FAD/BAC-TREM2 mice (FIG. 14B) were stained with CD68 and ThioS. Representative cortical images demonstrated elevated CD68 expression surrounding the plaques in 5×FAD/BAC-TREM2 mice.

FIG. 15 depicts the contextual memory function of mice from the cohort of BAC-TREM2×5×FAD (n=8-14 per genotype) was evaluated by contextual fear conditioning and is presented as percentage of time freezing. Power analysis was performed to ensure >80% confidence levels with the number of animals used. **p<0.01, *p<0.05.

FIG. 16, comprising FIG. 16A through FIG. 16H, depicts increased TREM2 gene dosage alters plaque-associated microglia morphology and ameliorates behavioral deficit in a second mouse model of AD (APPswe/PSEN1dE9 or APP/PS1). FIG. 16A through FIG. 16F depict representative confocal images from 11-month-old APP/PS1 (FIG. 16A-FIG. 16C) and APP/PS1; TREM2 (FIG. 16D-FIG. 16F) mice stained with anti-Iba1 and 6E10 antibodies. Bar, 25 μm. FIG. 16G depicts the contextual memory function was evaluated by contextual fear conditioning and is presented as percentage of time freezing. n=14-18 per genotype, ***p<0.001, **p<0.01.

FIG. 17, comprising FIG. 17A through FIG. 17B, depicts. Increased TREM2 gene dosage reduced plaque-associated microglia and Iba+ expression. FIG. 17A depicts matching brain section from 11 months old APP/PS1 and APP/PS1/TREM2 mice were immunostained with anti-Iba1 and 6E10 antibodies. Amyloid plaques in cortex were randomly selected and plaque-associated microglia were counted under 40× lens in a blinded manner. n for APP/PS1 and APP/PS1; TREM2=3 and 2, respectively. *p<0.05. FIG. 17B depicts random cortical fields with amyloid plaques were selected for confocal imaging with 40× lens. 6E10+ plaque area and the mean fluorescent intensity of Iba1+ immunostaining were measured with ImageJ. The data is expressed in mean fluorescent intensity per 10,000 μm2 of plaque area. n for APP/PS1 and APP/PS1; TREM2=3 and 2, respectively. **p<0.01.

FIG. 18 depicts a schematic of the methods used for generating human genomic regulatory models with engineering large human genomic transgenic (HGT) DNA construct (e.g. Bacterial Artificial Chromosome or BAC) to drive the expression of Microglia Expressed AD Genes (ME-AD) in mammalian animal and cell models. The ME-AD genes include nearly two dozen genes that are genome-wide significantly associated with LOAD and are also show enriched expression in the microglia in the brain (Yeh et al., 2017, Trends Mol Med, 23:512-533), and they may also include additional microglia-enriched genes that are significantly dysregulated in the brains of AD and other neurodegenerative disorders or models of these disorders (e.g. Deczkowska et al., 2018, Cell, 173:1073-1081). Human genomic constructs such as a BAC can be engineered to express different types of Reporters. (i). One version is fuse in-frame the Reporter with the N- or C-terminal coding sequence of ME-AD gene on the BAC, which is called HGTp. This version can express ME-AD protein fused with the Reporter protein from human genomic regulatory elements at transcriptional regulation (promoter, enhancers, suppressors, locus control regions, etc), RNA levels (e.g. splicing, RNA transport, RNA modifications, RNA stability, polyadenylation), and protein levels (protein synthesis, turnover, etc). (ii). Another version is to insert a Reporter coding gene (without its polyadenylation signals) in the 5′ untranslated region (UTR) of the ME-AD gene (e.g. within the exon 1) on the human genomic DNA fragment (e.g. BAC) hence the Reporter is expressed under the ME-AD gene regulation on the genomic DNA, e.g. transcriptional regulation (promoter, enhancers, suppressors, locus control regions, etc) RNA level regulation (e.g. splicing, RNA transport, RNA modifications, RNA stability, polyadenylation signals). These HGT models can be used for two purposes: (1). The mammalian genetic models can be used to screen for molecular therapeutics that regulate the ME-AD protein or RNA levels in vivo (e.g. microglia in the brain or myeloid cells in the non-central nervous system (CNS) tissues); and (2). Primary microglia or myeloid cells or cell lines can be either derived from the HGT mammalian models or directly generated using the HGT constructs in existing cell models (microglia- or myeloid-like). These cell models can be used to screen for molecular therapeutics that can alter the expression of ME-AD protein or RNA in the ex vivo cellular models.

FIG. 19 depicts a schematic of the methods used for generating Human Genomic Knockin (HGKI) cell models by gene targeting or genome editing to insert reporters into the endogenous ME-AD loci in human cells. Currently there are human cell models, including induced pluripotent stem cells (iPSCs) or myeloid cells that can be differentiated into microglia-like or myeloid-like cells. Moreover, there are immortalized human cell lines that exhibit molecular and phenotypic features of microglia and other myeloid cells, e.g. expressing ME-AD genes. Finally, there are other human cell lines, which may or may not be myeloid-like, but express substantial levels of certain ME-AD genes. Using homologous recombination based gene targeting or CRISPR/Cas based genome editing, reporters are introduced into the endogenous ME-AD locus in the aforementioned cell lines to develop the following two types of cell models. In one version, the Reporter is fused in-frame with the N- or C-terminal coding sequence of ME-AD gene in the endogenous loci, which is called HGKIp. This version expresses the ME-AD-Reporter fusion protein from human genomic regulatory elements at transcriptional regulation (promoter, enhancers, suppressors, locus control regions, etc) RNA levels (e.g. splicing, RNA transport, RNA modifications, RNA stability, polyadenylation), and protein levels (protein synthesis, turnover, etc). (ii). In another version the Reporter is inserted in the 5′ UTR region of the ME-AD gene in the endogenous loci of the cell model. In this version, the Reporter is expressed under the ME-AD gene regulation on the genomic DNA, e.g. transcriptional regulation (promoter, enhancers, suppressors, locus control regions, etc) RNA level regulation (e.g. splicing, RNA transport, RNA modifications, RNA stability, polyadenylation). The HGKI mammalian models can be derived from existing cell models (e.g. transformed microglia or myeloid-like cell lines, or microglia or myeloid-like cells derived from human embryonic stem cells, induced pluripotent stem cells, and other somatic cell types). These cell models can be used to screen for molecular therapeutics that can alter the expression of ME-AD protein or RNA in the ex vivo cellular models.

FIG. 20, comprising FIG. 20A and FIG. 20B, depicts the experimental design and validation of HGTpc-TREM2-GFP transgenic mice (also known as BAC-TREM2-GFP mice). FIG. 20A depicts a schematic diagram showing that the previously modified human TREM2 BAC, i.e. RP11-237K15 was further modified with deletion of key coding exons in TREML1, TREML2 and TREML4 (Lee et al., 2018, Neuron, 97:1032-1048) to insert in frame the coding sequence for Green Fluorescent Protein or GFP to the very last coding amino-acid of human TREM2 on the BAC (FIG. 18 and FIG. 19). FIG. 20A depicts exemplary experimental results demonstrating that the properly engineered BAC was used to generate BAC-TREM2-GFP transgenic mice, which showed selective expression of GFP reporter protein (fused with TREM2) only in a subset of microglia at the baseline (about 5.8% in the cortex and 8.7% in the hippocampus), but not in the neurons or astrocytes.

FIG. 21 depicts exemplary images depicting that the expression of the TREM2-GFP fusion protein is appropriately upregulated in disease-associated microglia (DAM; Deczkowska et al., 2018, Cell, 173:1073-1081) in 5×FAD/BAC-TREM2-GFP double transgenic mice at 5-month of age. 5×FAD mouse models show early accumulation (e.g. 3-4 month of age) of amyloid plaques and microglia activation as indicated by upregulation of Iba1 expression in the microglia. Prior RNA-seq studies showed murine Trem2 and human TREM2 are both upregulated in disease-associated microglia (DAM) in AD and other neurodegenerative disease models (Deczkowska et al., 2018, Cell, 173:1073-1081). Double transgenic mice carrying the 5×FAD and BAC-TREM2-GFP transgenes were generated, and at 5 month of age there is a proper upregulation of the TREM2-GFP fusion protein in the activated microglia (i.e. Iba1 high expressing microglia) of these mice, demonstrating the BAC-TREM2-GFP reporter can appropriately upregulate the GFP reporter in the DAM microglia.

FIG. 22, comprising FIG. 22A and FIG. 22B, depicts a schematic of the methods used for generation and initial characterization of HGTr-TREM2-NLuc BAC transgenic mice. FIG. 22A depicts a schematic demonstrating that the previously modified human TREM2 BAC (i.e. RP11-237K15 with deletion of key coding exons in TREML1, TREML2 and TREML4; Lee et al., 2018, Neuron, 97:1032-1048) was further modified to insert in the exon 1 5′ UTR region of TREM2 on the BAC the coding sequence for NLuc. Subsequently the BAC-TREM2-NLuc construct was injected into fertilized mouse embryos to generate BAC transgenic mouse lines expressing NLuc reporter protein from the human TREM2 genomic transgenes. The genomic construct will express NLuc (Hall et al., 2012, ACS Chem Biol, 7:1848-57) under the transcriptional regulatory elements of TREM2 on the BAC, and it also preserves all the splicing regions as well as 3′ UTR of TREM2, hence can report other RNA regulatory mechanisms for TREM2 (e.g. splicing, transport, stability). These mouse lines are called HGTr-TREM2-NLuc (or BAC-TREM2r-NLuc). FIG. 22B depicts exemplary experimental evidence demonstrating that brain extracts derived from the brain of two transgenic mouse lines exhibit high NLuc activities at P5, the age that TREM2 is expected to be expressed at high levels in the brain (Chertoff et al., 2013, PLoS One, 8:e72083).

### DETAILED DESCRIPTION OF THE INVENTION

The invention provides, in part, a novel set of strategies to screen for molecules that can regulate the levels of microglia or myeloid expressed genes, including disease-associated genes (ME-AD genes), in mammalian animal and cell models. The ME-AD genes include, but are not limited to, nearly two dozen genes that are genome-wide significantly associated with LOAD and show enriched expression in the microglia in the brain (Yeh et al., 2017, Trends Mol Med, 23:512-533), myeloid- or microglia-enriched genes that are significantly dysregulated in the brains of AD and other neurodegenerative disorders or are significantly dysregulated in models of these disorders (e.g. Deczkowska et al., 2018, Cell, 173:1073-1081) and genes expressed in microglia or other myeloid cells that are functionally linked (i.e. associated) with AD or other neurodegenerative disorders. ME-AD genes are implicated in the regulation of microglia function and in pathogenesis of AD and other related brain disorders.

In one embodiment, the invention relates to methods for identifying molecules that can affect the transcription, RNA splicing, trafficking and stability of ME-AD genes in normal conditions or in conditions of environmental or disease-related challenge.

In one embodiment, the invention relates to methods of screening of molecules that modulate (i.e., downregulate or upregulate) the expression levels of ME-AD genes in cell models, and molecules that may regulate the expression of ME-AD genes in vivo in intact animals, both in the CNS or in the peripheral tissues.

In one embodiment, the methods are applicable for identification of molecules that regulate ME-AD genes under the endogenous human DNA, RNA and protein regulatory context.

In one embodiment, the invention provides genetic tools that enable the screening of molecules regulating the levels of ME-AD genes both in cell models (e.g. primary microglia or transformed microglial/myeloid cell lines) and in vivo in transgenic animals.

### Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

An “allele” refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variants.” “polymorphisms,” or “mutations.”

As used herein, to “alleviate” a disease or disorder, such as AD, means reducing the frequency or severity of at least one sign or symptom of a disease or disorder.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

The term “derived from” is used herein to mean to originate from a specified source.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

A “fluorophore” is a component of a molecule which causes a molecule to be fluorescent. It is a functional group in a molecule which will absorb energy of a specific wavelength and re-emit energy at a specific wavelength. The amount and wavelength of the emitted energy depend on both the fluorophore and the chemical environment of the fluorophore. Fluorescein isothiocyanate (FITC), a reactive derivative of fluorescein, has been one of the most common fluorophores chemically attached to other, non-fluorescent molecules to create new fluorescent molecules for a variety of applications. Other historically common fluorophores are derivatives of rhodamine (TRITC), coumarin, and cyanine. Newer generations of fluorophores such as the CF dyes, the FluoProbes dyes, the DyLight Fluors, the Oyester dyes, the Atto dyes, the HiLyte Fluors, and the Alexa Fluors are also known in the art.

The term “gene” refers to a nucleic acid (e.g., DNA) sequence that includes coding sequences necessary for the production of a polypeptide, precursor, or RNA (e.g., mRNA). The polypeptide may be encoded by a full-length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 4 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

A “genome” is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

As used herein, the term “growth medium” is meant to refer to a culture medium that promotes growth of cells. A growth medium will often contain animal serum. In some instances, the growth medium may not contain animal serum.

As used herein, an “immunoassay” refers to any binding assay that uses an antibody capable of binding specifically to a target molecule to detect and quantify the target molecule.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated cell” refers to a cell which has been separated from other components and/or cells which naturally accompany the isolated cell in a tissue or mammal.

The term “modulate” or “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process.

A “nucleic acid” refers to a polynucleotide and includes poly-ribonucleotides and poly-deoxyribonucleotides. Nucleic acids according to the present invention may include any polymer or oligomer of pyrimidine and purine bases, e.g., cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, at least 8, at least 15 or at least 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”. As used herein, includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The term “reporter gene” or “reporter” is known in the art and as used in the present invention with respect to a DNA sequence means any DNA sequence encoding a peptide, a protein or a polypeptide or nucleic acid that can give rise to a signal that can be detected, traced, or measured. As used in the present invention with respect to a DNA sequence, “reporter” will generally means a cDNA sequence (although in some cases a reporter gene may have introns) that encodes a protein or polypeptide or nucleic acid that is used in the art to provide a measurable phenotype that can be distinguished over background signals. The product of said reporter gene may also be referred to a “reporter” and may be mRNA, a peptide, a polypetide, or protein, and may also be readily measured by any mRNA or protein quantification technique known in the art. “Reporter” may also refer to a tag or label that is affixed to a protein or peptide after it is expressed and may be any such tag or label known in the art. The reporter may, in a preferred embodiment, be a fluorophore.

The terms “patient,” “subject,” “individual,” and the like are used interchangeably herein, and refer to any animal, or cells thereof whether in vitro or in situ, amenable to the methods described herein. In certain embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “genetically modified” means an animal, the germ cells of which comprise an exogenous human nucleic acid or human nucleic acid sequence. By way of non-limiting examples a genetically modified animal can be a transgenic animal or a knock-in animal, so long as the animal comprises a human nucleic acid sequence.

As used herein, “knock-in” is meant a genetic modification that replaces the genetic information encoded at a chromosomal locus in a non-human animal with a different DNA sequence. As used herein, the terms “therapy” or “therapeutic regimen” refer to those activities taken to alleviate or alter a disorder or disease, such as AD, e.g., a course of treatment intended to reduce or eliminate at least one sign or symptom of a disease or disorder using pharmacological, surgical, dietary and/or other techniques. A therapeutic regimen may include a prescribed dosage of one or more drugs. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

To “treat” a disease as the term is used herein, means to reduce the frequency or severity of at least one sign or symptom of a disease or disorder, such as AD, experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention is based, in part on the identification of about two dozen AD GWAS significant genes with relatively enriched expression in microglia compared to many other brain cell types in human and mouse brains, and that these ME-AD genes could be key molecular targets to modify microglial function and to treat age-dependent neurodegenerative disorders including AD. Thus the invention provides ME-AD reporter systems, and methods of using the systems in reporter assays for screening for agents that modulate the expression or level of the ME-AD gene.

### Compositions

In one embodiment, the invention relates to constructs, cell lines and animal models for use in methods of identifying agents which alter the expression or activity of microglial expressed Alzheimer's disease associated (ME-AD) genes and gene products.

Microglial-expressing, Alzheimer's disease associated genes (ME-AD genes) include, but are not limited to, AD GWAS significant genes identified in Hansen et al., 2018, J Cell Biol, 217:459-472; Efthymiou and Goate, 2017, Mol Neurodegener, 12:43; and Wes et al., 2016, Glia, 64:1710-1732, the disclosures of which are incorporated herein in their entirety. Examples of such ME-AD genes include, but are not limited to, TREM2, DAP12 (TYROBP), CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN and MEF2C. APOE can also be considered one of the ME-AD genes as it is significantly upregulated in DAM microglia (Deczkowska et al., 2018, Cell, 173:1073-1081), and downregulation of APOE at certain stages in mouse models of AD could reduce plaque pathology (Huynh et al., 2017). Additional ME-AD genes may include those that are dysregulated in AD or other neurodegenerative disorders (e.g. DAM microglia genes; Deczkowska et al., 2018). An example of ME-AD gene in this category include Grn, a microglia-enriched gene linked to FTD (Baker et al., 2006, Nature, 442:916-919; Cruts et al., 2006, Nature, 442:920-924; van Swieten and Heutink, 2008, Lancet Neurol, 7:965-974), microglia function (Lui et al., 2016, Cell, 165:921-935), and protection against amyloid pathology in AD (Minami et al., 2014, Nat Med, 20:1157-1164).

In one embodiment, the invention relates to vectors and cells comprising a human genomic transgenic (HGT) reporter construct for use in methods of identifying agents which alter the expression or activity of microglial expressed AD genes. In one embodiment, an HGT reporter construct of the invention includes a nucleotide sequence encoding a ME-AD gene operably linked to one or more nucleotide sequences encoding a reporter molecule. In various embodiments, the reporter molecule may be operably linked to the N terminus or the C terminus of the ME-AD protein, or may internal within the ME-AD protein.

In one embodiment, the HGT construct of the invention is optimized for expression in a host cell. A promoter can be optimized based on the type of host cell or to optimize the signal to noise ratio for expression of the reporter.

In one embodiment, the HGT construct of the invention comprises a translational initiation sequence or enhancer, such as the so-called “Kozak sequence” (Kozak, J. Cell Biol. 108: 229-41 (1989)) or “Shine-Dalgarno” sequence.

In one embodiment, the invention relates cell lines and transgenic animals comprising a nucleotide sequence encoding a reporter sequence operably linked to a nucleotide sequence encoding a MD-AD protein for use in methods of identifying agents which alter the expression or activity of microglial expressed AD genes. In one embodiment, the sequence encoding the reporter molecule serves to label or tag a ME-AD gene. Therefore, in one embodiment, the invention provides ME-AD knockin cell lines and knockin animals comprising at least one ME-AD gene operably linked to one or more nucleotide sequences encoding a reporter molecule. In various embodiments, the reporter molecule may be operably linked to the N terminus or the C terminus of the ME-AD protein, or may internal within the ME-AD protein.

In one embodiment, the invention relates to cell lines and transgenic animals comprising a nucleotide sequence encoding a reporter molecule operably linked to promoter of an ME-AD gene for use in methods of identifying agents which alter the expression or activity of ME-AD genes. In one embodiment, the invention provides ME-AD cell lines and transgenic animals comprising at least one promoter region of a ME-AD gene operably linked to one or more nucleotide sequences encoding a reporter molecule. In various embodiments, the reporter molecule may be operably linked to the N terminus or the C terminus of the ME-AD protein, or may internal within the ME-AD protein.

A reporter molecule is a molecule, including polypeptide as well as polynucleotide, expression of which in a cell confers a detectable trait to the cell. In various embodiments, reporter markers include, but are not limited to, chloramphenicol-acetyl transferase (CAT), β-galactosyltransferase, horseradish peroxidase, luciferase, NanoLuc®, alkaline phosphatase, and fluorescent proteins including, but not limited to, green fluorescent proteins (e.g. GFP, TagGFP, T-Sapphire, Azami Green, Emerald, mWasabi, mClover3), red fluorescent proteins (e.g. mRFP1, JRed, HcRed1, AsRed2, AQ143, mCherry, mRuby3, mPlum), yellow fluorescent proteins (e.g. EYFP, mBanana, mCitrine, PhiYFP, TagYFP, Topaz, Venus), orange fluorescent proteins (e.g. DsRed, Tomato, Kusabria Orange, mOrange, mTangerine, TagRFP), cyan fluorescent proteins (e.g. CFP, mTFP1, Cerulean, CyPet, AmCyan1), blue fluorescent proteins (e.g. Azurite, mtagBFP2, EBFP, EBFP2, Y66H), near-infrared fluorescent proteins (e.g. iRFP670, iRFP682, iRFP702, iRFP713 and iRFP720), infrared fluorescent proteins (e.g. IFP1.4) and photoactivatable fluorescent proteins (e.g. Kaede, Eos, IrisFP, PS-CFP).

In one embodiment, two or more reporter markers are under control of the promoter sequence of the HGT construct or the ME-AD gene of the invention, to provide amplification of the signal. Therefore, in one embodiment, the invention provides HGT construct of the invention comprising a ME-AD gene sequence operably linked to nucleotide sequences encoding at least 1, at least 2, at least 3, at least 4, at least 5, or more than 5 reporter markers. In one embodiment, the invention provides cells or transgenic animals comprising a ME-AD gene sequence operably linked to nucleotide sequences encoding at least 1, at least 2, at least 3, at least 4, at least 5, or more than 5 reporter markers. In one embodiment, two or more tandem reporter markers are all the same (e.g. two tandem copies of luciferase.) Alternatively, two or more tandem reporter markers may be of different reporters.

HGT Constructs

The present invention includes a vector in which the ME-AD reporter construct of the present invention is inserted. The art is replete with suitable vectors that are useful in the present invention.

The expression of a ME-AD reporter construct is typically achieved by operably linking a nucleic acid sequence comprising a promoter to a nucleic acid sequence encoding a reporter molecule or portions thereof, and incorporating the construct into an expression vector. In one embodiment, the vectors to be used are suitable for replication and, optionally, integration in eukaryotic cells. Typical vectors contain transcription and translation terminators, initiation sequences, and other regulatory sequences useful for regulation of the expression of the desired nucleic acid sequence.

The ME-AD reporter construct of the invention can be cloned into a number of types of vectors. For example, the nucleic acid can be cloned into a vector including, but not limited to a BAC, a plasmid, a phagemid, a phage derivative, an animal virus, and a cosmid. Vectors of particular interest include expression vectors, replication vectors, BACs, and sequencing vectors.

A number of large-capacity viral based systems have been developed for gene transfer into mammalian cells. For example, adenoviral vectors have been developed that provide a platform for transgene delivery of up to 38 kB and herpes simplex virus (HSV) vectors can deliver up to 150 kb of transgenic DNA. A selected gene can be inserted into a vector and packaged in viral particles using techniques known in the art. The recombinant virus can then be isolated and delivered to cells of the subject either in vivo or ex vivo. Large capacity retroviral systems, including, but not limited to, foamy virus (FV) vectors, are also known in the art. For example, vectors derived from retroviruses such as the FV vectors are suitable tools to achieve long-term gene transfer since they allow long-term, stable integration of a transgene and its propagation in daughter cells.

In one embodiment, a ME-AD reporter vector comprises an origin of replication capable of initiating DNA synthesis in a suitable host cell. In one embodiment, the origin of replication is selected based on the type of host cell. For instance, it can be eukaryotic (e.g., yeast) or prokaryotic (e.g., bacterial) or a suitable viral origin of replication may be used.

In one embodiment, a ME-AD reporter vector comprises a selection marker gene to facilitate identification and selection of expressing cells from the population of cells sought to be transfected or infected through viral vectors. In other aspects, the selectable marker may be carried on a separate piece of DNA and used in a co-transfection procedure. Selectable marker genes may be flanked with appropriate regulatory sequences to enable expression in the host cells.

A selection marker sequence can be used to eliminate host cells in which the ME-AD reporter vector has not been properly transfected. A selection marker sequence can be a positive selection marker or negative selection marker. Positive selection markers permit the selection for cells in which the gene product of the marker is expressed. This generally comprises contacting cells with an appropriate agent that, but for the expression of the positive selection marker, kills or otherwise selects against the cells. For suitable positive and negative selection markers, see Table I in U.S. Pat. No. 5,464,764.

Examples of selection markers also include, but are not limited to, proteins conferring resistance to compounds such as antibiotics, proteins conferring the ability to grow on selected substrates, proteins that produce detectable signals such as luminescence, catalytic RNAs and antisense RNAs. A wide variety of such markers are known and available, including, for example, a Zeocin™ resistance marker, a blasticidin resistance marker, a neomycin resistance (neo) marker (Southern & Berg, J. Mol. Appl. Genet. 1: 327-41 (1982)), a puromycin (puro) resistance marker; a hygromycin resistance (hyg) marker (Te Riele et al., Nature 348:649-651 (1990)), thymidine kinase (tk), hypoxanthine phosphoribosyltransferase (hprt), and the bacterial guanine/xanthine phosphoribosyltransferase (gpt), which permits growth on MAX (mycophenolic acid, adenine, and xanthine) medium. See Song et al., Proc. Nat'l Acad. Sci. U.S.A. 84:6820-6824 (1987). Other selection markers include histidinol-dehydrogenase, chloramphenicol-acetyl transferase (CAT), dihydrofolate reductase (DHFR), β-galactosyltransferase and fluorescent proteins such as GFP.

Expression of a fluorescent protein can be detected using a fluorescent activated cell sorter (FACS). Expression of β-galactosyltransferase also can be sorted by FACS, coupled with staining of living cells with a suitable substrate for β-galactosidase. A selection marker also may be a cell-substrate adhesion molecule, such as integrins, which normally are not expressed by the host cell. In one embodiment, the cell selection marker is of mammalian origin, for example, thymidine kinase, aminoglycoside phosphotransferase, asparagine synthetase, adenosine deaminase or metallothionien. In one embodiment, the cell selection marker can be neomycin phosphotransferase, hygromycin phosphotransferase or puromycin phosphotransferase, which confer resistance to G418, hygromycin and puromycin, respectively.

Suitable prokaryotic and/or bacterial selection markers include proteins providing resistance to antibiotics, such as kanamycin, tetracycline, and ampicillin. In one embodiment, a bacterial selection marker includes a protein capable of conferring selectable traits to both a prokaryotic host cell and a mammalian target cell.

Negative selection markers permit the selection against cells in which the gene product of the marker is expressed. In some embodiments, the presence of appropriate agents causes cells that express “negative selection markers” to be killed or otherwise selected against. Alternatively, the expression of negative selection markers alone kills or selects against the cells.

Such negative selection markers include a polypeptide or a polynucleotide that, upon expression in a cell, allows for negative selection of the cell. Illustrative of suitable negative selection markers are (i) herpes simplex virusthymidine kinase (HSV-TK) marker, for negative selection in the presence of any of the nucleoside analogs acyclovir, gancyclovir, and 5-fluoroiodoamino-Uracil (FIAU), (ii) various toxin proteins such as the diphtheria toxin, the tetanus toxin, the cholera toxin and the pertussis toxin, (iii) hypoxanthine-guanine phosphoribosyl transferase (HPRT), for negative selection in the presence of 6-thioguanine, (iv) activators of apoptosis, or programmed cell death, such as the bc12-binding protein (BAX), (v) the cytidine deaminase (codA) gene of E. coli. and (vi) phosphotidyl choline phospholipase D. In one embodiment, the negative selection marker requires host genotype modification (e.g. ccdB, tolC, thyA, rpsl and thymidine kinases.)

In accordance with the present invention, the selection marker usually is selected based on the type of the cell undergoing selection. For instance, it can be eukaryotic (e.g., yeast), prokaryotic (e.g., bacterial) or viral. In such an embodiment, the selection marker sequence is operably linked to a promoter that is suited for that type of cell.

In one embodiment, an HGT construct of the invention comprises a transcription termination sequence. A typical transcriptional termination sequence includes a polyadenylation site (poly A site). In one embodiment, a poly A site is the SV40 poly A site. These sequences may be located in the ME-AD reporter construct 3′ to a reporter gene sequence or a selection marker sequence.

In one embodiment, an HGT construct of the invention comprises one or more termination/stop codon(s) in one or more reading frames at the 3′ end of a reporter marker sequence or selection marker sequence, such that translations of these sequences are terminated at the stop codon(s).

ME-AD Reporter Construct

In one embodiment, a ME-AD reporter construct of the invention provides a nucleic acid molecule comprising at least one promoter sequence of a ME-AD gene, operably linked to at least one reporter marker.

In one embodiment, a ME-AD gene promoter comprises a region 5′ to a ME-AD gene. In one embodiment, the gene is one of TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN or MEF2C.

In one embodiment, a nucleotide sequence encoding at least one reporter molecule in inserted in-frame downstream of the translational start codon of TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN orMEF2C.

### Genome Editing Compositions

In one embodiment, the invention provides for integration of the reporter system of the invention into a host cell through use of a genome editing system. A series of programmable nuclease-based genome editing technologies have developed (see for example, Hsu et al., Cell 157, Jun. 5, 2014 1262-1278), including, but not limited to, meganucleases, zinc finger nucleases (ZFNs), transcription activator-like effector-based nucleases (TALENs) and CRISPR-Cas systems (see e.g. Platt et al., Cell 159(2), 440-455 (2014); Shalem et al., Science 3 84-87 (2014); and Le Cong et al., Science 339, 819 (2013)) or alternative CRISPR systems. Genome editing systems have a wide variety of utilities including modifying (e.g., deleting, inserting, translocating, inactivating, activating, repressing, altering methylation, transferring specific moieties) a target polynucleotide in a multiplicity of cell types. In one embodiment, a CRISPR-Cas system is used to integrate the reporter constructs of the invention into a host genome. The CRISPR-Cas system can include at least one guide RNA (gRNA) targeted to a target nucleic acid sequence, and a CRISPR-associated (Cas) peptide form a complex to induce insertion of the reporter constructs at the targeted nucleic acid sequence.

In one embodiment, the target polynucleotide is a DNA molecule. DNA molecules include, but are not limited to, genomic DNA molecules, extrachromosomal DNA molecules, conjugative plasmids and exogenous DNA molecules.

In general, “CRISPR-Cas system” or “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system are derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In some embodiments, the site of reporter integration is determined by the CRISPR-Cas system guide RNA. In general, a “CRISPR-Cas guide RNA” or “guide RNA” refers to an RNA that directs sequence-specific binding of a CRISPR complex to the target sequence. Typically, a guide RNA comprises (i) a guide sequence that has sufficient complementarity with a target polynucleotide sequence to hybridize with the target sequence and (ii) a trans-activating cr (tracr) mate sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In the context of formation of a CRISPR complex, a “target sequence” or “a sequence of a target DNA” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides or DNA/RNA hybrid polynucleotides. In some embodiments, a target sequence is located in the nucleus or cytoplasm of a cell. In some embodiments, the target sequence may be within an organelle of a eukaryotic cell, for example, mitochondrion or chloroplast.

In some embodiments, the CRISPR-Cas domain comprises a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, orthologs thereof, or modified versions thereof. In some embodiments, the Cas protein has DNA or RNA cleavage activity. In some embodiments, the Cas protein directs cleavage of one or both strands of a nucleic acid molecule at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence.

Cells

In one embodiment, the invention relates to cells or cell lines containing a ME-AD reporter construct of the invention. Methods of introducing and expressing genes in a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). In one embodiment, the method of introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, the present invention provides a cell or population of cells modified to comprise a ME-AD reporter construct of the invention. In one embodiment, the cells are prokaryotic cells. In one embodiment, cells are eukaryotic cells. In one embodiment, a cell is a mammalian cell, such as a murine or human cell. The target cell may be a somatic cell or a germ cell. The germ cell may be a stem cell, such as embryonic stem cells (ES cells), including murine embryonic stem cells. The target cell may be an induced pluripotent stem cell (iPSC) or a myeloid cell that can be differentiated into microglia-like or myeloid-like cells.

The target cell may be chosen from commercially available mammalian cell lines. The target cell may be a primary cell isolated from a subject. A target cell may be any type of diseased cell, including cells with abnormal phenotypes that can be identified using biological or biochemical assays. In one embodiment, a cell may be an HEK293 cell. In one embodiment, a cell may be a myeloid cell line that expresses TREM2 and its signaling partner DAP12 (TYROBP) (Satoh et al., 2012, Cell Mol Neurobiol, 32:337-343), such as THP-1 cells.

The cells of the invention and cells derived therefrom can be derived from, inter alia, humans, primates, rodents and birds. In one embodiment, the cells of the invention are derived from mammals, especially mice, rats and humans. In one embodiment, cells may be either wild-type or genetically modified cells.

The cells of the present invention, whether grown in suspension or as adherent cell cultures, are grown in contact with culture media.

In one embodiment, culture media used in the present invention comprises a basal medium, optionally supplemented with additional components. Basal medium is a medium that supplies essential sources of carbon and/or vitamins and/or minerals for the cells. The basal medium is generally free of protein and incapable on its own of supporting self-renewal/symmetrical division of the cells. Media formulations that support the growth of cells include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's salt base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with nonessential amino acids), and the like.

It is further recognized that additional components may be added to the culture medium. Such components include, but are not limited to, antibiotics, antimycotics, albumin, growth factors, amino acids, and other components known to the art for the culture of cells. Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml. However, the invention should in no way be construed to be limited to any one medium for culturing the cells of the invention. Rather, any media capable of supporting the cells of the invention in tissue culture may be used.

Typical substrates for culture of the cells in all aspects of the invention are culture surfaces recognized in this field as useful for cell culture, and these include surfaces of plastics, metal, composites, though commonly a surface such as a plastic tissue culture plate, widely commercially available, is used. Such plates are often a few centimeters in diameter. For scale up, this type of plate can be used at much larger diameters and many repeat plate units used. For high throughput assays multi-well plates, having 6, 12, 24, 48, 96 or more wells can be used.

The culture surface may further comprise a cell adhesion protein, usually coated onto the surface. Receptors or other molecules present on the cells bind to the protein or other cell culture substrate and this promotes adhesion to the surface and promotes growth. In certain embodiments, the cultures of the invention are adherent cultures, i.e. the cells are attached to a substrate.

### Modulators of ME-AD Gene Expression

In one embodiment, the invention provides modulators of at least one ME-AD gene identified using the compositions and methods of the invention.

In various embodiments, to determine whether an agent results in the modulation (e.g., an increase or decrease in expression) of a ME-AD reporter construct, the level of expression of the reporter construct is compared with the level of at least one comparator control, such as a positive control, a negative control, a historical control, a historical norm, or the level of another reference molecule in the biological sample.

In various embodiments of the assays of the invention, the level of expression is determined to be elevated when the level of expression is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator control.

In various embodiments of the assays of the invention, the level of expression is determined to be elevated when the level of expression is increased by at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator control.

In various embodiments of the assays of the invention, the level of expression is determined to be decreased when the level of expression is decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator control.

In various embodiments of the assays of the invention, the level of expression is determined to be decreased when the level of expression is decreased by at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator control.

The ME-AD reporter constructs, cell lines and transgenic animals of the invention can be used for identifying agents that modulate expression of an ME-AD gene using any appropriate method for detecting the level of expression of a reporter marker. Appropriate methods include both high-throughput and low-throughput methods.

In various embodiments of the invention, methods of measuring the level of expression of a reporter marker include, but are not limited to, an immunochromatography assay, an immunodot assay, a luminescence assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

### Transgenic Animals

In one embodiment, the invention provides transgenic animals whose germ cells and somatic cells contain a transgene encoding one or more human ME-AD gene, with the transgene including all regulatory elements of the human ME-AD gene necessary for microglial expression of the transgene in the transgenic animal, and/or for human patterns of expression of the transgene in the transgenic animal.

In one embodiment, the transgenic animal of the invention is a transgenic mouse. The production of transgenic mice can be carried out in view of the disclosure provided herein and in light of techniques known to those skilled in the art, such as described in U.S. Pat. No. 5,767,337 to Roses et al.; U.S. Pat. No. 5,569,827 to Kessous-Elbaz et al.; and U.S. Pat. No. 5,569,824 to Donehower et al. (the disclosures of which are incorporated by reference herein in their entirety).

Mice of the invention are preferably characterized by exhibiting expression of the ME-AD reporter proteins in microglial cells and other myeloid cells thereof, and/or a human pattern of ME-AD reporter expression in neuronal and non-neuronal cells (i.e. microglia). By “human pattern” of expression is meant that the pattern of distribution of the ME-AD reporter proteins is expressed in mouse microglial cells thereof, and proper (i.e., similar to the endogenous gene expression) low level or lack of expression in other cell types in the brain, such as neurons, oligodendrocytes, astrocytes.

Thus, mice of the invention are useful for the study of the effects of test conditions and compounds on the expression or activity of ME-AD genes. The ability of a compound to modulate a ME-AD gene may be determined or screened by administering a test compound to an animal of the invention and then monitoring the animal for the expression of a ME-AD reporter marker.

Animals may be administered a test compound by any suitable means, such as by parenteral injection, oral administration, inhalation administration, transdermal administration, retroorbital injections, etc.

In one embodiment, transgenic mice of the invention contain a transgene of an entire human ME-AD gene operably linked to a reporter marker. In one embodiment, transgenic mice of the invention contain a reporter marker that operably linked to an endogenous mouse ME-AD gene or endogenous mouse ME-AD promoter.

In one embodiment, the “knockout” mice or “null” mice of the invention are mice whose germ and somatic cells contain an inactive mouse ME-AD gene or disrupted mouse ME-AD gene, wherein Exon 1 (or other suitable segment) of said mouse ME-AD gene is deleted and replaced with an expression cassette, said expression cassette including a heterologous gene (e.g., a gene encoding a marker such as a luciferase reporter) operably associated with a promoter (e.g., an inducible or constitutively active promoter). Thus, the mouse ME-AD gene is “disrupted” by the presence of the heterologous gene. This disrupted ME-AD gene is then unable to program the expression of functional mouse ME-AD proteins.

The transgene inserted into animals of the invention (transgenic animals) is, in general, one that encodes a human ME-AD gene. The transgene may be a genomic sequence (that is, one that includes both introns and exons) that encodes a human ME-AD protein. The gene may include one or more mutations to the sequence thereof.

In one embodiment, the entire human ME-AD gene is added to a mouse cell to make human-ME-AD transgenic mice. In one embodiment, these mice are mated to obtain a mouse that expresses only human ME-AD protein, along with the reporter marker.

### Methods

The invention is based in part on the development of a method for screening using the ME-AD reporter constructs, cell lines and transgenic animals of the invention and the use of the screening method to identify new therapeutic agents for AD or a disease or disorder associated with a ME-AD gene. In various embodiments, the invention relates to methods of using ME-AD reporter constructs or cells modified with ME-AD reporter constructs to identify compounds or treatments that affect a biological pathway or process. In one embodiment, the invention relates to methods of treating AD or a disease or disorder associated with a ME-AD gene through administration of a compound or treatment that modulates a ME-AD gene to a subject in need thereof.

### Methods of Screening

In one embodiment, the invention relates to a method of screening for the effect of an agent, condition or treatment on the expression or activity of an ME-AD gene.

In one embodiment, modified cells and transgenic animals of the invention can be used to screen for drugs or compounds that regulate (e.g., activate or inhibit) expression or activity of an ME-AD gene. A drug or compound library may be applied to a modified cell of the invention, in which the ME-AD reporter construct is inserted within an exon or intron of the ME-AD gene of interest and/or under control of the endogenous promoter and regulatory elements (transcription, splicing, RNA trafficking and stability) of a ME-AD gene of interest, to screen for candidates that may regulate the expression of the promoter (e.g. transcription) and/or affect the expression levels of the gene (e.g. mRNA levels).

In one embodiment, the invention relates to the use of ME-AD reporter cells or animals in methods of screening for down-regulators (i.e., repressors) of the ME-AD gene of interest, the method comprising contacting a population of ME-AD cells with an agent, measuring the level of expression of a reporter molecule, detecting an decrease in expression of the reporter molecule as compared to a comparator control, and identifying the agent as a repressor of the ME-AD gene expression levels based on the decrease in expression of the reporter molecule. In one embodiment, a comparator control is a population of ME-AD cells that has not been contacted with the agent.

In one embodiment, the invention relates to the use of ME-AD reporter cells or animals in methods of screening for up-regulators (i.e., activators) of the ME-AD gene of interest, the method comprising contacting a population of ME-AD cells with an agent, measuring the level of expression of a reporter molecule, detecting an increase in expression of the reporter molecule as compared to a comparator control, and identifying the agent as an activator of the ME-AD gene expression levels based on the increase in expression of the reporter molecule. In one embodiment, a comparator control is a population of ME-AD cells that has not been contacted with the agent.

In some embodiments, the modulator is one or more molecules selected from the group consisting of a small interfering RNA (siRNA), a small guide RNA (gRNA), a microRNA, an antisense or sense nucleic acid, a ribozyme, an expression vector encoding a transdominant negative mutant, an antibody, a peptide, a chemical compound and a small molecule. In some embodiments, the modulator is one or more compound selected from the group consisting of a chemical compound, a protein, a peptidomimetic, an antibody, a nucleic acid molecule.

### Methods of Modulating ME-AD Genes

In one embodiment, the invention relates to methods of modulating a ME-AD gene through administration of an activator or repressor identified by the method of screening as having an effect on the ME-AD gene. In one embodiment, the invention relates to methods of modulating one or more of TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN and MEF2C through administration of an activator or repressor of one or more of TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN and MEF2C.

### Methods of Treatment

The invention relates to methods of treating a disease or disorder associated with one or more ME-AD gene comprising administering to a subject in need thereof a modulator of one or more ME-AD gene identified by the method of screening as having an effect that is beneficial to the treatment of the disease or disorder. In one embodiment, the disease or disorder is AD. In one embodiment, an activator or repressor of an ME-AD gene is an activator or repressor of at least one of TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN and MEF2C.

Diseases or disorders associated with a ME-AD gene that can be treated by the disclosed methods and compositions include, but are not limited to, Alzheimer's disease (AD) and other dementias, Parkinson's disease (PD) and PD-related disorders, Frontotemporal Dementia (FTD) Amyotrophic lateral sclerosis (ALS), Huntington's disease (HD), spinocerebellar ataxias (SCAs), prion disease, spinal muscular atrophy (SMA), Lewy body dementia (LBD), multisystem atrophy, primary progressive aphasia, multiple sclerosis (MS), ischemic stroke, traumatic brain injury, HIV-associated dementia, and other neurodegenerative, neurological and psychiatric diseases or disorders that involve dysregulated or functionally altered ME-AD genes.

The disorder or disease associated with a ME-AD gene can be treated by administration of therapeutic agent comprising a modulator of at least one of TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN and MEF2C alone or in combination with another treatment or therapeutic agent. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing test systems which are well known to the art.

At least one suitable unit dosage form having the therapeutic agent(s) of the invention, can be administered by a variety of routes including parenteral, including by intravenous and intramuscular routes, as well as by direct injection into the subject.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the exemplary embodiments of the present invention, and are not to be construed as limiting in any way the remainder of the disclosure.

### Example 1

### Elevated TREM2 Gene Dosage Reprograms Microglia Responsivity and Ameliorates Pathological Phenotypes in Alzheimer's Disease Models

Recent studies have begun to unravel the functional roles of TREM2 in molecular, cellular, and animal models that are informative to AD. Substantial effort has been devoted to identify TREM2 ligands. TREM2 has been shown to bind anionic and zwitterionic lipids found on damaged neurons (Wang et al., 2015, Cell, 160:1061-1071) and AD-associated proteins APOE and Clusterin (Atagi et al., 2015, J. Biol. Chem., 290:26043-26050; Bailey et al., 2015, J. Biol. Chem., 290:26033-26042; Yeh et al., 2016, Neuron, 91:328-340). In APP mouse models, Trem2 plays a role in clustering and activating microglia around Aβ plaques (Jay et al., 2015, J. Exp. Med., 212:287-295, Wang et al., 2015, Cell, 160:1061-1071). The impact of Trem2 deficiency on amyloid plaque formation is dynamic and complex. At an early disease stage, the plaque load is reduced in an AD model crossed to Trem2 knockout mouse (Jay et al., 2015, J. Exp. Med., 212:287-295), while in more advanced disease stages, the plaque load increased in multiple AD models (Jay et al., 2017, J. Neurosci., 37:637-647, Wang et al., 2015, Cell, 160:1061-1071). Moreover, Trem2 in plaque-associated microglia plays an important role in plaque compaction and insulation to reduce the neuritic toxicity of fibrillary Aβ (Wang et al., 2016, J. Exp. Med., 213:667-675; Yuan et al., 2016, Neuron, 90:724-739). Thus far, most Trem2 studies in disease models in vivo have used loss-of-function mutants, yet very little is known about the impact of increased Trem2 expression under its genomic regulation on normal brain function and in disease responses.

In the current study, TREM2 function was interrogated in microglia at baseline and in AD disease mice through a gain-of-function genetic approach that mimics gene-dosage increase in the germline, which commonly occurs during evolution (Zarrei et al., 2015, Nat. Rev. Genet., 16:172-183). Although in vitro studies suggest that overexpression of TREM2 in microglia promotes DAP12 signaling, phagocytosis of dead neurons, and suppression of pro-inflammatory responses (Takahashi et al., 2005, J. Exp. Med., 201:647-657), currently there are no in vivo studies to assess the impact of increased TREM2 gene dosage on microglial function and AD pathogenesis. To directly test the effect of upregulating TREM2 in AD mouse models in vivo, bacterial artificial chromosome (BAC)-mediated transgenesis was used to insert extra copies of the human TREM2 genomic DNA segment into the mouse genome, resulting in elevated TREM2 expression selectively in microglia in the brain. Increase in TREM2 gene dosage was found to reprogram microglia responsivity and ameliorate disease phenotypes in multiple amyloid deposition mouse models of AD.

This study provides a rigorous and detailed analysis of the phenotypic impact of increased TREM2 gene dosage, through BAC-mediated transgenesis, on a variety of phenotypes in two AD mouse models. The experiments demonstrate that TREM2 BAC can selectively drive reporter transgene expression in microglia in the mouse brain, and elevate human TREM2 RNA and protein expression. In the context of AD mice, such elevated TREM2 expression led to reduction of the amyloid plaque load and a shift of plaque composition from the fibrillary toward more compact and inert types. Brain transcriptomic and network analyses revealed partial rescuing effects at the transcriptome-wide level in 5×FAD/BAC-TREM2 (referred to as 5×FAC/TREM2) mice. Detailed examination of cortical transcriptomes by RNA-sequencing revealed known disease-associated microglial genes showing an interesting reprogramming response in the 5×FAD/TREM2 mouse brains compared to that in 5×FAD; there is a selective downregulation of a subset of reactive microglial genes (i.e., TD1) and upregulation of a second subset (TD2). Such molecular reprogramming of microglia in the diseased brain is supported by evidence of the number, morphology, and phagocytic marker expression of the plaque-associated microglia in the 5×FAD/TREM2 mice. Functional studies in vitro showed TREM2 gene-dosage increase augmented microglia phagocytic activity, a phenotype opposite to that of Trem2 deficiency. The experiments also showed evidence for reduced neuritic pathology and improved memory task in the AD models with elevated TREM2 gene dosage. Together, this study reveals that elevated TREM2 gene dosage can mediate microglia reprogramming, reduced neuropathology, and improved cognitive performance in AD mouse models.

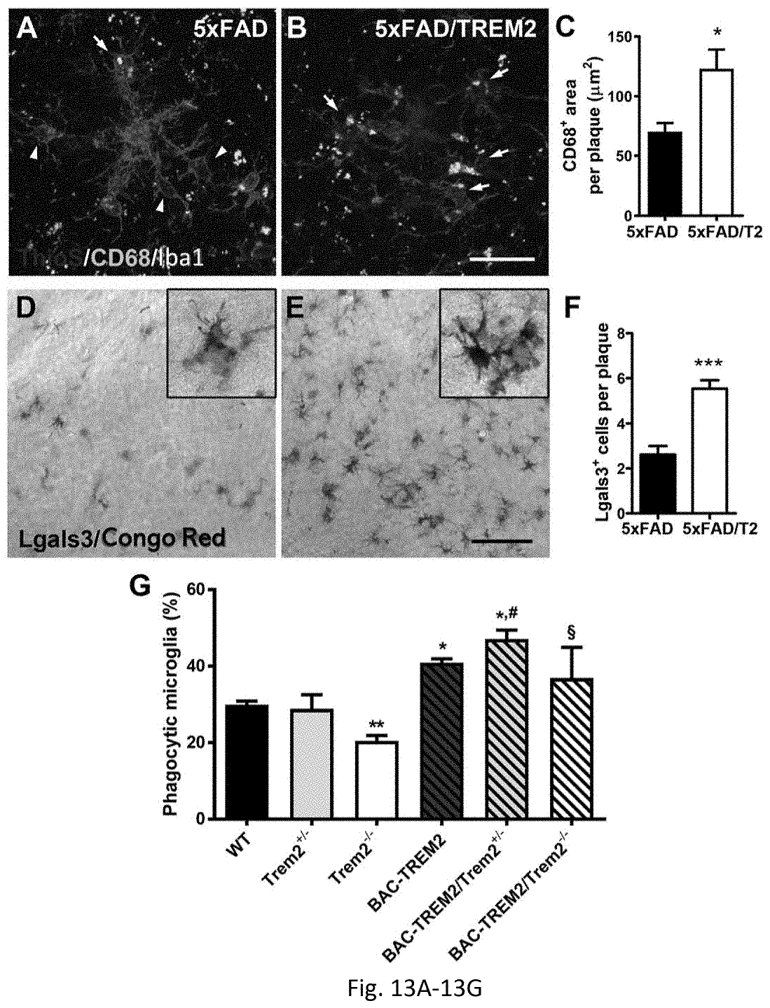

A key strength of this study is the mouse genetic construct design, which allows for precisely addressing the increase of TREM2 gene dosage on microglial function and disease-related phenotypes in vivo. BAC transgenes are known to drive more accurate, endogenous-like transgene expression (Gong et al., 2002, Genome Res., 12:1992-1998, Yang et al., 1997, Nat. Biotechnol., 15:859-865) and are suitable for studying the effects of gene-dosage increase in intact animals (Yang et al., 1999, Nat. Genet., 22:327-335). The use of human BAC transgene allows for the study of TREM2 function in the human TREM2 genomic DNA, RNA, and protein context, which could be relevant to investigating disease variants in vivo (Jordan et al., 2015, Nature, 524:225-229). Strong evidence was obtained that the BAC transgene is properly expressed in microglia cells using the novel BAC-TREM2-GFP reporter line and that TREM2 is functional in complementing the Trem2 deficiency in the in vitro phagocytosis assay (FIG. 13G). An important aspect of the BAC transgene design is the deletion of essential exons in three other TREM-like genes on the BAC, which are known to have important innate immunity function (Colonna, 2003, Nat. Rev. Immunol., 3:445-453; Ford and McVicar, 2009, Curr. Opin. Immunol., 21:38-46), and may play distinct roles in AD (Carrasquillo et al., 2017, Alzheimers Dement., 13:663-673). Thus, in order to draw strong conclusions on the role of TREM2 gene-dosage increase on the microglial and disease phenotypes in AD mouse models, one would have to genetically abolish the expression of TREM-like molecules from the BAC transgene, a strategy that is implemented in the BAC-TREM2 mice.

Another important question addressed in this study is whether increased TREM2 gene dosage, hence its expression levels under genomic regulation, is beneficial or harmful in the disease process. Trem2 deficiency induces dynamic changes, but an eventual increase in plaque and neuritic pathology occurs (Jay et al., 2017, J. Neurosci., 37:637-647; Wang et al., 2015, Cell, 160:1061-1071; Wang et al., 2016, J. Exp. Med., 213:667-675; Yuan et al., 2016, Neuron, 90:724-739). In addition to amyloid-induced pathology, two recent studies also demonstrated that Trem2 deficiency altered reactive microgliosis and proinflammantory responses of microglia in two tauopathy mouse models (Bemiller et al., 2017, Mol. Neurodegener., 12:74; Leyns et al., 2017, Proc. Natl. Acad. Sci. USA, 114:11524-11529), which are reminiscent of those observed in the APP models (Jay et al., 2015, J. Exp. Med., 212:287-295; Jay et al., 2017, J. Neurosci., 37:637-647; Wang et al., 2015, Cell, 160:1061-1071). However, its impact on tau-induced pathology remains discrepant in these models. Since deletion of Trem2 in mice blocks the proper activation of microglia in neurodegenerative diseases (Keren-Shaul et al, 2017, Cell, 169:1276-1290; Krasemann et al., 2017, Immunity, 47:566-581, Immunity, 47:566-581, Wang et al., 2015, Cell, 160:1061-1071), it is difficult to predict a priori whether TREM2gene-dosage increase could broadly or subtly alter the microglial transcriptome response, and what impact it would exert on the disease process. This study provides strong evidence for a partial, but significant, rescuing effect of TREM2gene-dosage increase on the transcriptomic phenotypes, amyloid pathology, and behavioral impairment in AD mouse models. The transcriptome-wide rescue/exacerbation analyses and WGCNA network analyses show unbiased evidence for partial normalization of the disease-associated gene expression in the 5×FAD/TREM2 mice, with reduction of expression of microglial and astrocyte module genes and upregulation of neuronal and synaptic genes.

In addition, this study sheds some light on the kinetics of TREM2 level increase that could be effective in exerting beneficial effects in AD mice. Prior studies (e.g., Keren-Shaul et al, 2017, Cell, 169:1276-1290; Krasemann et al., 2017, Immunity, 47:566-581) and the current one show elevated Trem2 expression occurs at relatively late disease stages. Moreover, an increase in secreted extracellular fragment of TREM2 appears to be a CSF biomarker of disease progression in AD (Suárez-Calvet et al., 2016, EMBO Mol. Med., 8:466-476). These findings raise the question of whether elevated TREM2 during disease pathogenesis in AD is beneficial in modulating disease progression. This study showed that BAC-TREM2 mice have elevated TREM2 levels as early as 2 months of age. Thus, without being bound by theory, it is believed that upregulation of TREM2 levels early in the AD mouse brains, prior to the upregulation of hundreds of other reactive microglial genes, may be more effective in reprogramming the microglial responsivity to ameliorate the disease.

This study provides tantalizing clues to the TREM2-mediated microglial reprogramming. For example, several dichotomous cellular and molecular phenotypes are demonstrated between BAC-TREM2 and Trem2-deficient microglia in mouse models of AD (Jay et al., 2017, J. Neurosci., 37:637-647; Ulrich et al., 2017, Neuron, 94:237-248; Yuan et al., 2016, Neuron, 90:724-739). These include the compaction of amyloid plaques, the ramification of plaque-associated microglial processes, the expression of certain activated microglial markers, and phagocytosis. These microglial phenotypes may help to pinpoint the molecular function that is rate limited by the levels of Trem2 across a broad dynamic range, hence could be more proximal to the direct molecular function of TREM2 in microglia in response to the brain disease environment.

The molecular analyses provide candidates that may underlie the phenotypic changes in microglia response and overall disease phenotypes in the AD mice crossed to BAC-TREM2. Although both downregulated (TD1) and upregulated microglial genes (TD2) could play a role in the TREM2 gene-dosage effect in the AD mice, without being bound by theory, it is believed that the upregulated microglial genes are prime candidates to investigate. A subset of TD2 genes show transcriptional changes that are bidirectional in Trem2 knockout and our BAC-TREM2 mice crossed to 5×FAD, which could be transcriptional targets that are positively regulated by TREM2 levels (or signaling), a hypothesis that can be tested. Moreover, multiple TD2 genes have known functions that are consistent with Trem2 effects in myeloid cells. Lgals3 is a critical regulator of microglia activation (Rotshenker, 2009, J. Mol. Neurosci., 39:99-103), and it facilitates phagocytosis (Sano et al., 2003, J. Clin. Invest., 112:389-397). Interestingly, Lgals3 is shown to be an “eat-me” signal linking phagocytic receptor Mertk to its cargo (Caberoy et al., 2012, J. Cell. Physiol., 227:401-407). Spp1 regulates cytokine expression and promotes microglia survival (Rabenstein et al., 2016, J. Neuroimmunol., 299:130-138), a function similar to that of Trem2 (Yeh et al., 2017, Trends Mol. Med., 23:512-533). Postn is shown to be required for alternative (less inflammatory) activation of microglia in brain tumors (Zhou et al., 2015, Nat. Cell Biol., 17 (2015), pp. 170-182). The latter finding is consistent with the enrichment in TD2 of genes for “negative regulators of T cell activation”. These molecular changes may help to explain the overall altered reactivity of microglia in BAC-TREM2-expressing AD mice. Future studies are needed to investigate whether and how the new molecular candidates as well as known TREM2 associated molecules could be tapped to reprogram microglial responsivity upon TREM2 gene-dosage increase.

This study provides strong genetic evidence that increased TREM2 gene dosage can modify microglial transcriptional programs and morphological and functional responses in the brain of AD mouse models. Such microglia molecular reprogramming led to reduced plaque load and enhanced plaque compaction, reduced dystrophic neurites, and improved behavioral outcomes. This study supports early boosting of TREM2 levels or signaling to prevent the onset or reduce the severity of pathological microglial response and overall disease phenotypes in neurodegenerative diseases including AD.

The materials and methods are now described

Generation of BAC Transgenic Mice

RP11-237K15 BAC contains the human TREM2 gene, as well as surrounding TREML1, TREML2, and TREML4 genes. The fidelity of the TREM2 gene was confirmed with Sanger sequencing of PCR products covering the entirety of the gene. TREML1, TREML2, and TREML4 genes were deleted with 4 sequential modification steps using RecA-based shuttle vector plasmids described previously (Yang et al., 1997, Nat. Biotechnol., 15:859-865; Gong et al., 2002, Genome Res., 12:1992-1998; Gong and Yang, 2005, Curr. Protoc. Neurosci., Chapter 5). Exons 1-3 with proximal promoter region were deleted from TREML4, excising a majority of the protein coding sequence. Due to concern for a downstream in-frame ATG site in TREML1, Exons 5-6 were deleted along with exons 1-2 and the proximal promoter region, abolishing 80% of TREML1's protein-coding sequences. For TREML2, exon 2-3 were deleted, resulting in a frameshift and early stop site in exon 4. All the BAC modification products were confirmed using established methods (e.g., PCR, restriction mapping, etc; Gong and Yang, 2005, Curr. Protoc. Neurosci., Chapter 5). TREM2-GFP BAC was modified from TREM2 BAC by introducing the EGFP sequence to the 3′ end of TREM2 before the stop codon with the methods described above. The modified BAC DNA was prepared according to our published protocols and microinjected into FvB fertilized oocytes. BAC-TREM2 and BAC-TREM2-GFP mice were maintained in the FvB/NJ background.

Animal Breeding and Husbandry

5×FAD and APPswe/PSEN1dE9 (APP/PS1) mice were purchased from the Jackson Laboratory (JAX) and crossed to BAC-TREM2 mice in FvB/NJ inbred background. Thus, all genotypes of mice used in the current study were generated and analyzed in the F1 hybrid background (C56BL6J; FvB/NJ F1), which is suitable for phenotypic study of genetically engineered mutant mice (Silva et al., 1997, Neuron, 19:755-759). Animals were housed in standard mouse cages under conventional laboratory conditions, with constant temperature and humidity, 12 h/12 h light/dark cycle and food and water ad libitum. All animal studies were carried out in strict accordance with National Institutes of Health guidelines and approved by the UCLA Institutional Animal Care and Use Committees. Matched number of mice in both genders were used in the study. Age and the number (n) of mice used are as indicated in the individual experiments and figures.

Tissue Collection and Sample Preparation

Mice were anesthetized with pentobarbital and perfused with ice-cold PBS. Brains were bisected. The right hemispheres were immediately submerged in ice-cold DEPC/PBS and cortices and hippocampi were carefully dissected out under a dissection microscope. Dissected tissues were snap frozen in dry ice and stored in −80° C. before further processing. The left hemispheres were fixed in 4% PFA/PBS overnight followed by submergence in 30% sucrose before freezing. Coronal sections (40 μm) were obtained using a cryostat and stored in cryopreserve solution at −20° C.

For preparing the samples for RNA sequencing and biochemistry, dissected brain tissues were homogenized and aliquoted as described previously (Cramer et al., 2012, Science, 335:1503-1506). In brief, cortical and hippocampal tissues from one hemisphere were homogenized in tissue homogenization buffer (250 mM sucrose, 20 mM Tris at pH 7.4, 1 mM EDTA, and 1 mM EGTA in DEPC-treated water) and centrifuged at 5000×g for 10 min at 4° C. Supernatants were aliquoted and stored at −80° C.

Aβ ELISA

Homogenates of cortical samples were subjected to sequential extraction using DEA (0.4% diethylamine in 100 mM NaCl) and FA (formic acid, >95%) solutions as described previously (Cramer et al., 2012, Science, 335:1503-1506). Concentration of soluble (DEA) and insoluble (FA) Aβ fractions were measured by ELISA using anti-Aβ₁₋₁₆ (6E10) as a capturing antibody. Specific Aβ species were detected by anti-Aβ₄₀-HRP and anti-Aβ₄₂-HRP antibodies with chromogenic substrate TMB (ThermoFisher). Absorbance at 650 nm was read on a Spectramax colorimetric plate reader (Molecular Devices).

Immunohistochemistry and Image Analysis

Coronal sections were blocked in the blocking buffer (3% BSA, 2% normal goat serum and 0.3% Triton X-100 in PBS) for 1 hour at room temperature and then incubated with primary antibodies at 4° C. overnight. Incubation in secondary antibodies was performed for 2 h at room temperature before mounting on slides with Prolong Diamond anti-fade mountant (ThermoFisher). Aβ plaques were visualized by ThioS and Congo Red staining or by immunostaining using anti-Aβ antibodies 6E10 and 4G8. For plaque number and categorization, 3 matched coronal sections/mouse spacing out across 1 mm (0.5 mm apart) were stained with 6E10 and ThioS. Z stack 20× images covering 30 μm thickness were taken on Zeiss LSM510 confocal microscopeand analyzed using ImageJ. Pixels with <1% of max intensity were discarded as background and were not counted as a part of the plaque. All images were preprocessed using the same threshold setting prior to analysis. For microglial morphology, Z stack 63× images of 50-55 overlapping optical slices aligned along the center of the plaques were collected from matched cortical regions. More than 12 plaques per genotype from 3 gender matched animals were taken. Morphology of all plaque-associated microglia in the images was analyzed using the FilamentTracer feature in Imaris 9.0 (Bitplane). The image acquisition and quantification described above were performed in a blinded manner.

RNA Purification and mRNA Sequencing

Total RNA was extracted using RNeasy kit (QIAGEN). Library preparation and RNA sequencing were performed by the UCLA Neuroscience Genomics Core (UNGC). Libraries were prepared using the Illumina TruSeq RNA Library Prep Kit v2 and sequenced on an Illumina HiSeq4000 sequencer using strand-specific, paired-end, 69-mer sequencing protocol to a minimum read depth of 30 million reads per sample. Reads were aligned to mouse genome mm10 using the STAR aligner (Dobin et al., 2013, Bioinformatics, 29:15-21) with default settings. Read counts for individual genes were obtained using HTSeq.

Human-specific TREM2 reads were obtained by aligning to the human reference genome (build GRCh38) reads that failed to align to the mouse genome (build mm10). Mouse-specific Trem2 reads were obtained in similar way. Mapped reads were quantified by the htseq-count tool (Anders et al., 2015, Bioinformatics, 31:166-169). TREM2 counts were divided by the library size per million to determine the counts per million (CPM) TREM2 level. Homer (Heinz et al., 2010, Mol. Cell, 38:576-589) makeTagDirectory (parameters: -format sam -flip -sspe) and makeUCSCfile (parameters: -fragLength given -o auto -raw) functions, bedtools (Quinlan and Hall, 2010, Bioinformatics, 26:841-842) and bedGraphToBigWig tools were used to create CPM bigwig tracks for visualization onto the UCSC genome browser.

Differential Expression Analysis

For outlier removal and for network analysis using WGCNA, mRNA profiles were retained whose observed counts are 5 or more in at least one-quarter of the samples and raw counts were transformed using variance stabilization. Outlier samples were removed as described (Oldham et al., 2012, BMC Syst. Biol., 6:63) using the Euclidean distance-based sample connectivity Z.k threshold of −6. This procedure resulted in the removal of a single sample (7 month old, female 5×FAD).

For DE testing and network analysis, individual observation weights were constructed as follows. Tukey bi-square-like weights λ are calculated for each (variance-stabilized) observation x, as

λ=(1−u2)2,

where u=min(1,|x−m|/(9MAD)), and m and MAD are median and median absolute deviation of the observations of the gene.

For each gene, MAD is adjusted such that (1) 10^{th} percentile of the weights λ is at least 0.1 (that is, the proportion of observations with coefficients<0.1 is less than 10%) (Langfelder and Horvath 2012, J. Stat. Softw., 46) and (2) for each individual time point and genotype, 40^{th} percentile of the weights λ is at least 0.9 (that is, at least 40% of the observation have a high coefficient of at least 0.9).

DE testing was carried out in R using package DESeq2 (Love et al., 2014, Genome Biol., 15:550) version 1.16.1.

DESeq2 models observed counts using Negative Binomial General Linear Models with dispersion estimated from data. Wald test was used to for significance calculations, and independent filtering was disabled. For differential expression testing between genotypes, sex was used as a covariate. Genotype-sex interactions were tested using models with genotype×sex terms (with genotype and sex turned into binary indicator variables).

For each genotype contrast or interaction, DE tests result in gene-wise Z statistics (fold changes divided by their standard errors). A “rescue/exacerbation” plot assesses overall similarity of genome-wide effects of two genotype contrasts using a scatterplot of their gene-wise DE Z statistics. A positive linear trend (correlation) indicates that the effects of the two genotype contrasts are broadly similar, whereas a negative correlation indicates broadly opposing effects. Although other measures are possible, the correlation value can be used as a measure of similarity.

Consensus Weighted Gene Co-Expression Network Analysis

Consensus Weighted Gene Co-expression Network Analysis (WGCNA) was carried out essentially as described previously (Langfelder and Horvath, 2007, BMC Syst. Biol, 1:54, Langfelder and Horvath, 2008, BMC Bioinformatics, 9:559). Since the experimental design contains two variables of interest (age and genotype) with strong effects on expression, a consensus network analysis (Langfelder and Horvath, 2007, BMC Syst. Biol, 1:54) of two datasets was carried out: data from WT, 5×FAD and 5×FAD/TREM2 genotypes at 2, 4, and 7 months, and data from the same genotypes at 4 and 7 months adjusted for age. The rationale is that a consensus analysis identifies modules that group together genes correlated in both datasets, i.e., both with respect to time point as well as genotype. The TREM2 genotype was left out of the network analysis since it is overall not different from WT. Rather, the network analysis was focused on the effects of BAC-TREM2 in the 5×FAD background (5×FAD versus 5×FAD/TREM2). Weighted correlation was used with individual sample weights determined as described above and the “signed hybrid” network in which negatively correlated genes are considered unconnected.

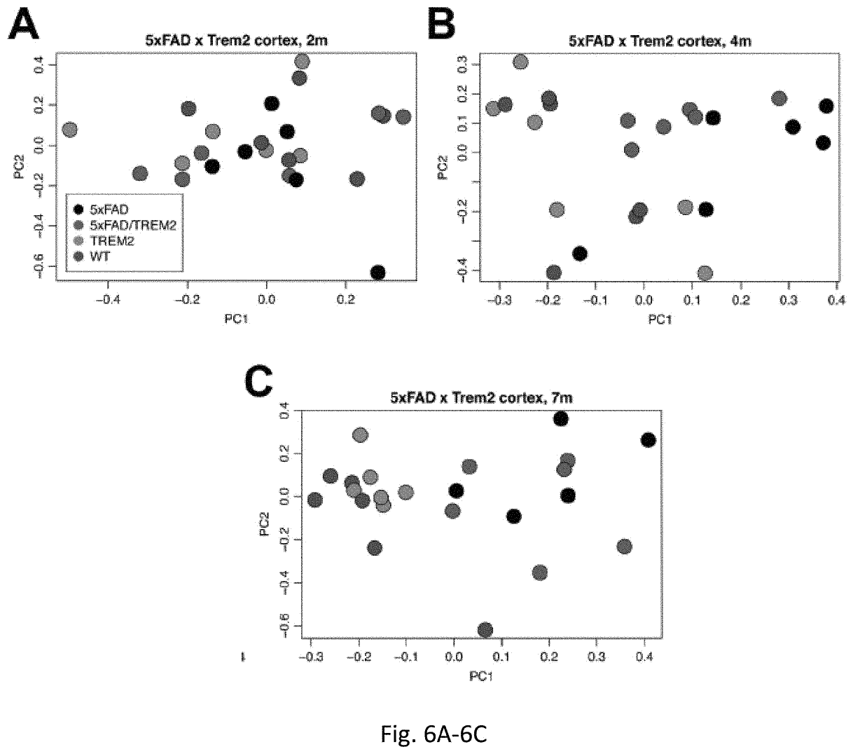

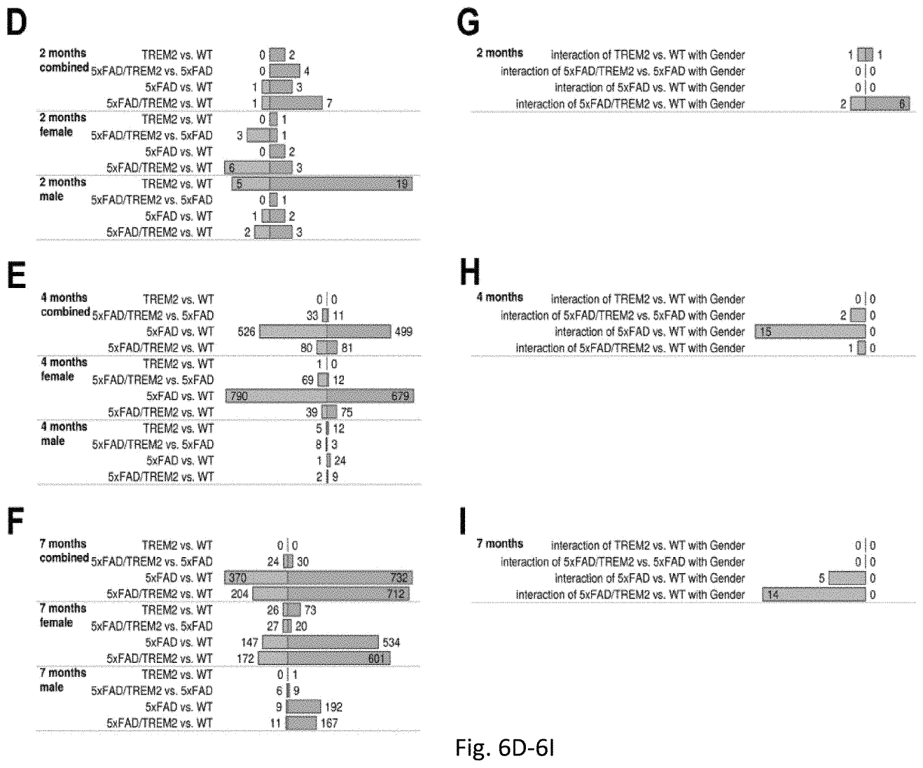

This analysis identified 28 co-expression modules ranging from 52 to 1767 genes per module (FIG. 6). Since genes in each module are co-expressed, it is advantageous to represent each module by a single representative expression profile (i.e., the module eigengene, which explains most of the variance of the module genes) (Horvath and Dong, 2008, PLoS Comput. Biol., 4:e1000117). Eigengenes were tested for DE between genotypes using standard linear models with sex as a covariate. Module eigengenes allow one to define a continuous (“fuzzy”) measure of membership of all genes in all modules (Horvath and Dong, 2008, PLoS Comput. Biol., 4:e1000117, Langfelder et al., 2016, Nat. Neurosci., 19:623-633). Genes with high fuzzy module membership in a module are called intramodular hub genes for the module.

Gene Set Enrichment Calculations

The R package anRichment was used to calculate the enrichment of DE genes and WGCNA modules in a large collection of reference gene sets that includes Gene Ontology (GO) terms, KEGG pathways, literature gene sets collected in the userListEnrichment R function (Miller et al., 2011, BMC Bioinformatics, 12:322), Molecular Signatures Database gene sets (Subramanian et al., 2005, Proc. Natl. Acad. Sci. USA, 102:15545-15550), aging gene sets from Enrichr (Chen et al., 2013, BMC Bioinformatics, 14:128) and other gene sets. In particular, microglia-relevant gene sets were collected from several recent articles (Butovsky et al., 2014, Nat. Neurosci., 17:131-143; Wang et al., 2015, Cell, 160:1061-1071; Gokce et al., 2016, Cell Rep., 16:1126-1137; Galatro et al., 2017, Nat. Neurosci., 20:1162-1171; Krasemann et al., 2017, Immunity, 47:566-581, Keren-Shaul et al, 2017, Cell, 169:1276-1290). Fisher exact test was used to evaluate overlap significance.

Primary Microglial Culture and Phagocytosis Assay

Primary microglia were isolated from the brains of neonatal mice at postnatal days 2-3 using a mild trypsinization protocol as previously described (Lee et al., 2012, J. Biol. Chem., 287:2032-2044). For phagocytosis assay, purified microglia were maintained in DMEM/F-12 (ThermoFisher) containing 2% heat-inactivated fetal bovine serum(FBS) and 1% penicillin/streptomycin plated at a density of 250,000 cells/well in 24-well plates for 3-5 days before further experiments. The culture media was replenished with serum-free DMEM/F12 overnight. Cells were incubated with BSA (0.5 mg/ml in PBS)-preblocked microsphere (1 μm, Alexa 488-conjugated; ThermoFisher) for 30 min, followed by extensive washing with PBS and fixation with 4% paraformaldedyde. After fixation, cells were washed with PBS and collected for analysis using an LSR II flow cytometer (BD Biosciences).

Behavioral Tests

Open-field exploration tests were performed for WT and BAC-TREM2 mice at 10 months of age (n=11 per genotype, with matched gender ratio) using our established protocols (Wang et al., 2014, Nat. Med., 20:536-541). Open-field testing was performed during the dark phase of the 12 h/12 h light-dark cycle.

Contextual fear conditioning test was performed with minor adjustment as describe previously (Curzon et al., 2009, J. J. Buccafusco (Ed.), Methods of Behavior Analysis in Neuroscience, CRC Press) and conducted in the Behavioral Testing Core (BTC) at UCLA. In brief, mice were handled daily for a week prior to the behavior test. In the training phase, mice were placed individually in the conditioning chamber to explore the environment freely for 2 minutes before the first unconditioned stimulus (US: 0.75 mA, 2 seconds) was delivered. The animals were exposed to 2 US's with an intertrial interval of 3 minutes. After the last shock, the mice were left in the chamber for another 1 minute and then placed back in their home cages. Retention tests were performed 24 hours later. Each mouse was returned to the same chamber for measuring the percent of time frozen and number of freezes. No shocks are given during the test session. Both training and testing procedures were videotaped and the freezing behavior was measured by an automated tracking system (Med Associates).

Statistical Analyses

Statistics for transcriptomic analyses were described as above. Other quantitative results, unless otherwise specified, were analyzed using one-way ANOVA with Tukey's post-hoc analysis or unpaired t test to determine the pvalue. Morphological and behavioral studies were subjected to Power analysis to determine the biological replicates (n) needed to reach >80% confidence level. n for individual experiments could be found in the results and figure legends.

The results of the experiments are now described

Generation of BAC-TREM2 Mice to Increase TREM2 Gene Dosage Under Endogenous Human Regulatory Elements

To increase Trem2 gene dosage, a BAC transgenic approach was used, which can increase the expression of genes on the BAC under genomic regulation (Yang et al., 1997, Nat. Biotechnol., 15:859-865; Yang et al., 1999, Nat. Genet., 22:327-335). A human TREM2 BAC was used for several reasons: first, human TREM2 and murine Trem2 are highly homologous (77% protein homology) and hence should have evolutionarily conserved function. Second, human TREM2 BAC is likely to preserve regulation of the human TREM2 gene expression at baseline and in disease state (Wilson et al., 2008, Science, 322:434-438). Finally, the human TREM2 protein may contain residues distinct from murine Trem2 that could be relevant to the future studies of disease variants (Jordan et al., 2015, Nature, 524:225-229).

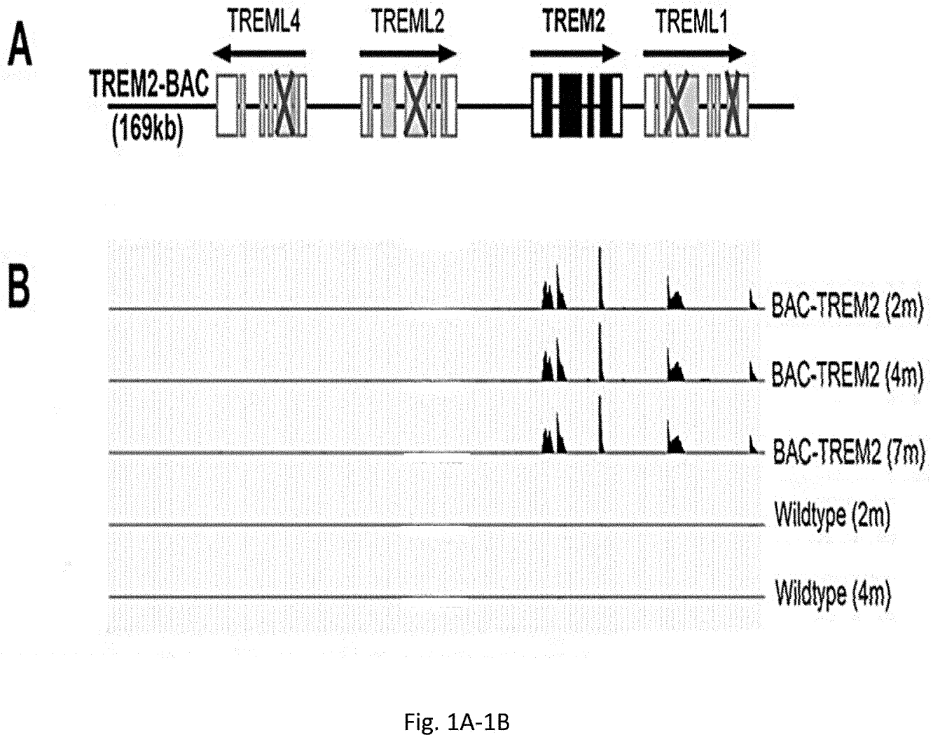

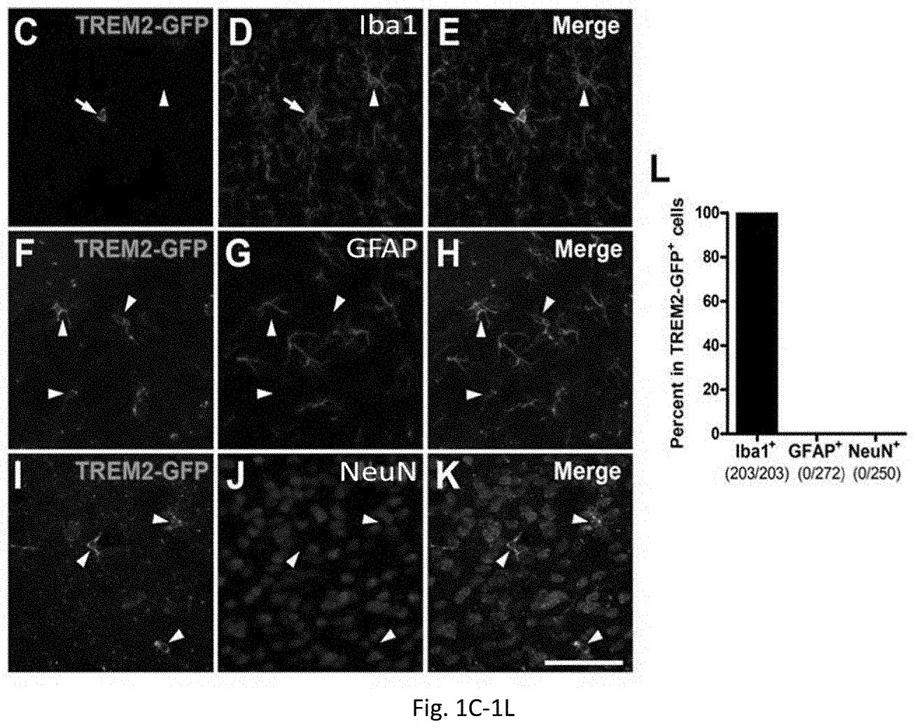

To generate human TREM2 BAC transgenic mice, BAC (RP11-237K15) was chosen, which encompasses the TREM2 coding region as well as surrounding genomic regions (>50 kb on each side) with conserved gene regulatory elements (Gong et al., 2003, Nature, 425:917-925). Since this BAC contains three other TREM-like genes, TREML1, TREML2, and TREML4, which likely serve critical innate immune functions (Colonna, 2003, Nat. Rev. Immunol., 3:445-453), their overexpression may confound the interpretation of TREM2 gene-dosage studies in vivo. To ensure the BAC only overexpresses TREM2, sequential BAC modification steps were used to delete key coding exons of these TREM-like genes on the BAC (FIG. 1A). The properly engineered BAC was used to generate BAC TREM2 transgenic founders in the FvB/NJ inbred background. Two independent BAC TREM2 founders (A and B) gave germline transmission of their transgenes, and genomic qPCR was used to estimate the transgene copy number to be 1-2 copies.

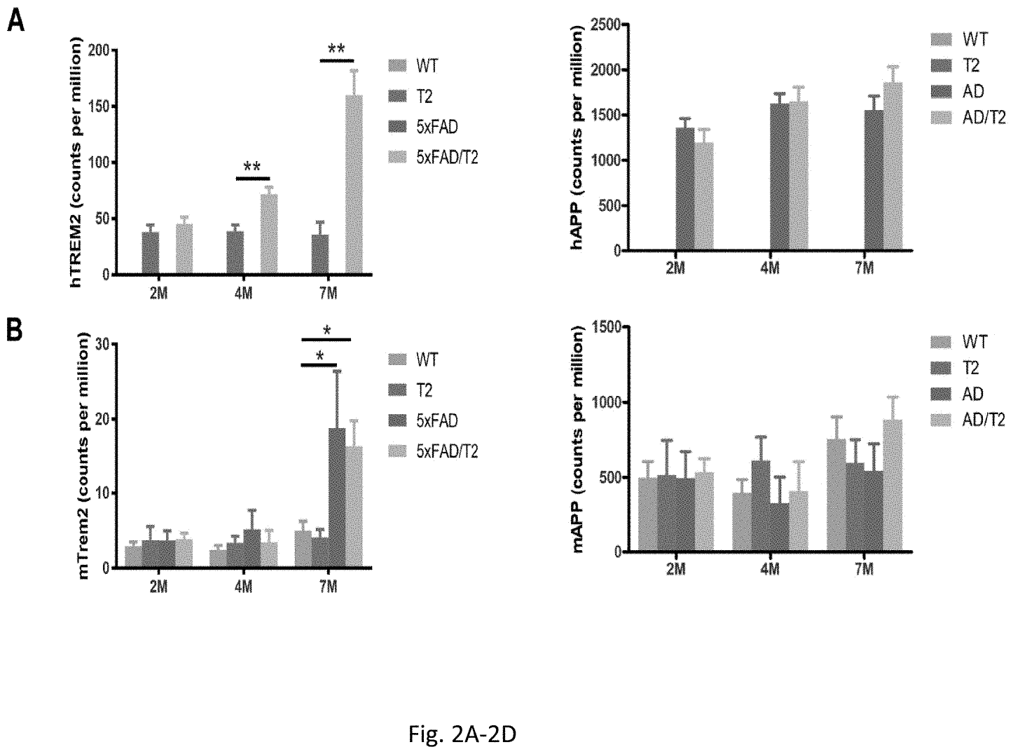

Next, the BAC TREM2 line A (referred to as BAC-TREM2) was selected to confirm the proper expression of human TREM2 RNA and protein. RNA sequencing (RNA-seq) of cortices from BAC-TREM2 and wild-type (WT) mice was used to identify unique reads of the mouse and human genomes covering murine Trem2 or human TREM2 genes (FIG. 1B). The human TREM2transcripts are only found in BAC-TREM2 mouse brains but not in WT brains at all ages tested. Importantly, consistent with the genetic design, reads mapping to human TREML1, TREML2, and TREML4 were not observed among the BAC-TREM2 transcripts (FIG. 1B). BAC-TREM2 mice expressed murine Trem2 transcripts at levels comparable to WT mice (FIG. 2).

To verify the cell-type-specific expression of the TREM2 transgene, available human TREM2 antibodies were used for immunostaining but did not generate robust signals. This might have been due to low baseline of TREM2 expression or to poor antibody specificity, as reported before (Jay et al., 2017, J. Neurosci., 37:637-647). Thus a BAC-TREM2-GFP reporter mouse line was engineered and created, which used the same TREM2 BAC as BAC-TREM2 but express TREM2 protein with a C-terminal GFP fusion. Double immunostaining of brain sections from BAC-TREM2-GFP mice demonstrated that the TREM2-GFP transgene was exclusively expressed in the Iba1+ microglia, but not in astrocytes or neurons (FIGS. 1CE-1KN). Only a subset of Iba1+ cells were GFP+ (about 5.8% in the cortex and 8.7% in the hippocampus) (FIGS. 1CE-1LG and 3A-3C). This is consistent with the low levels of TREM2 expression in the homeostatic microglia in the healthy brain (Keren-Shaul et al, 2017, Cell, 169:1276-1290; Krasemann et al., 2017, Immunity, 47:566-581). In summary, the novel BAC-TREM2-GFP reporter line demonstrates that the genomic regulatory elements on the human TREM2 BAC confer microglia-specific TREM2 expression in vivo.

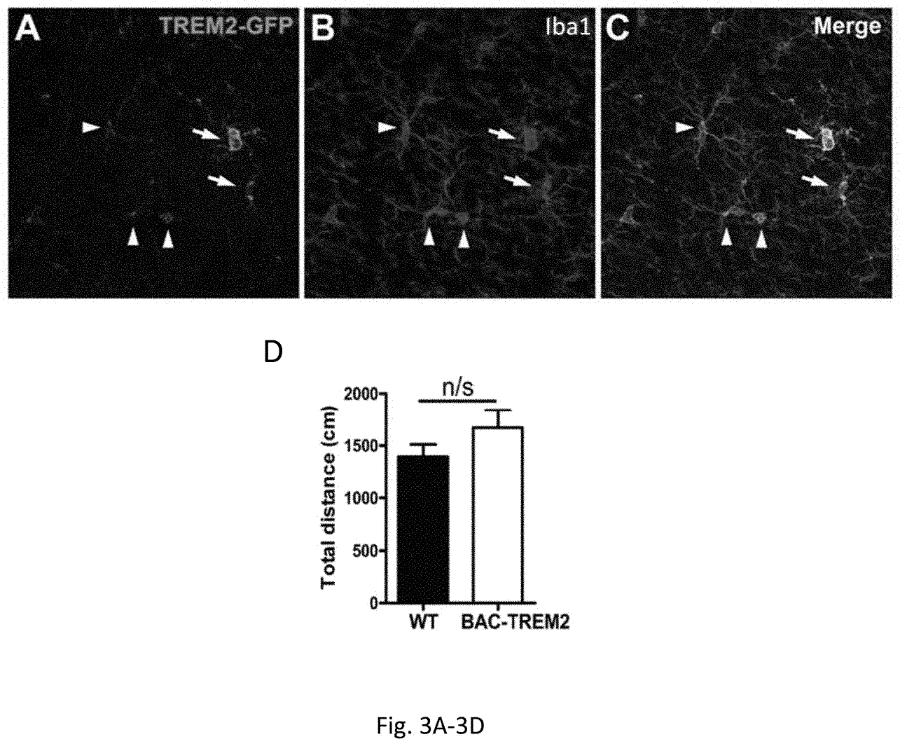

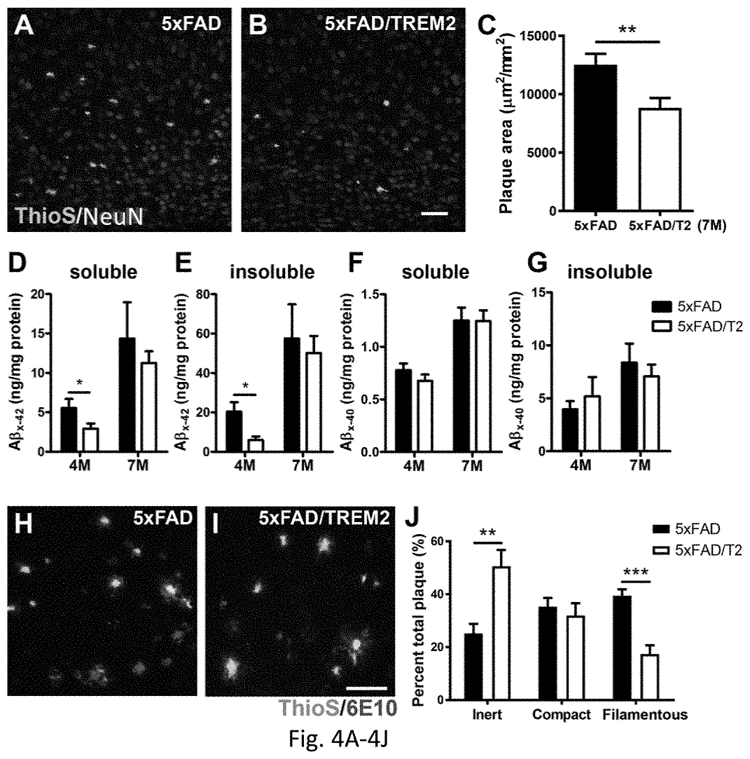

Microglia are known to have important functions in the brain, such as synaptic pruning in the hippocampus (Stephan et al., 2012, Annu. Rev. Neurosci., 35:369-389). However, BAC-TREM2 mice did not exhibit any detectable locomotion deficits (FIG. 3D). Together, these results suggested that BAC-mediated increase in TREM2 expression does not elicit overt brain functional deficits in mice.

Increased TREM2 Gene Dosage Reduces Amyloid Pathology in AD Mice

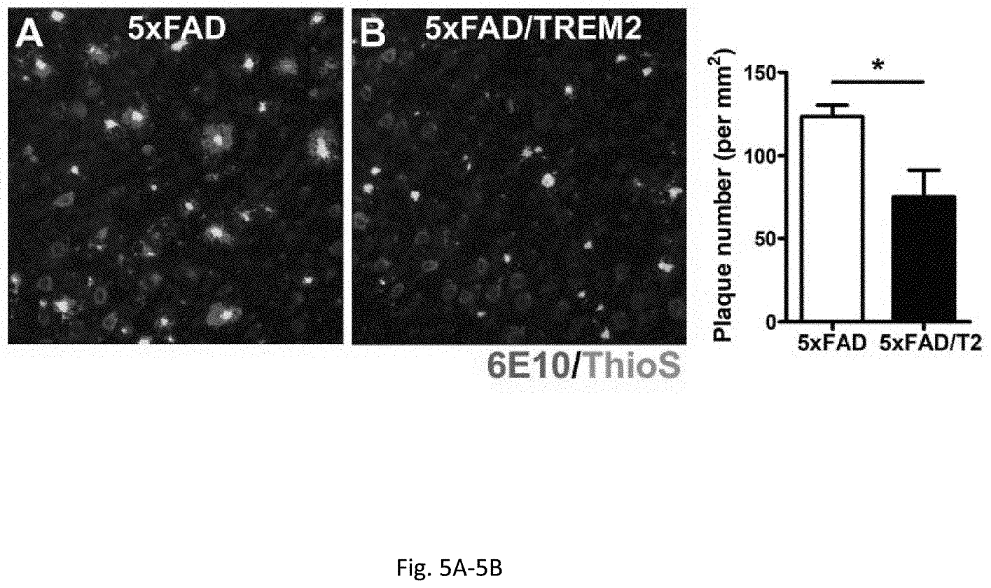

To address whether increased TREM2 gene dosage could alter disease-associated microglial function and other AD-related phenotypes, BAC-TREM2 were bred to 5×FAD mice (carrying 5 familial APP and PSEN1 mutations), which is an aggressive mouse model of amyloid deposition in AD (Oakley et al., 2006, J. Neurosci., 26:10129-10140). Staining of amyloid plaques with Thioflavin S (ThioS) on cortical sections from 7-month-old 5×FAD and 5×FAD/BAC-TREM2 (termed 5×FAD/TREM2 or 5×FAD/T2) mice revealed a significant reduction of amyloid plaque load in 5×FAD/TREM2 mice (FIGS. 4A-4C and 5). Next, the levels of soluble and insoluble Aβ₄₀ and Aβ₄₂ was measured in cortical lysates using ELISA and both the soluble and insoluble Aβ₄₂ were significantly decreased in 5×FAD/TREM2 mice at 4 months of age (FIGS. 4D and 4E). However, the difference in Aβ levels was less pronounced and no longer statistically significant at 7 months of age. No significant differences of Aβ₄₀ levels, the minor Aβ species, were detected in this AD mouse model, at both ages (FIGS. 4F and 4G). In order to examine whether the reduction of amyloid load resulted from alteration of APP expression, the human-specific APP transcript reads were examined in the RNA sequencing and it was found that the transgene was expressed at comparable levels in 5×FAD and 5×FAD/TREM2 cortices (FIG. 2C). Thus, the decreased plaque burden is likely due to TREM2-mediated changes in microglia-plaque interactions.

Recent studies have reported that Trem2 deficiency may disrupt the microglial barrier function in limiting the diffusion of fibrillary Aβ deposits (Wang et al., 2016, J. Exp. Med., 213:667-675; Yuan et al., 2016, Neuron, 90:724-739). To examine whether increased TREM2 expression alters the plaque property, distinct forms of plaques in the cortex (Yuan et al., 2016, Neuron, 90:724-739) were categorized and quantified. Aβ plaques in 5×FAD/TREM2 mice significantly shifted in composition toward the more inert form (strong ThioS⁺ with minor 6E10 staining) and less filamentous form (diffused 6E10⁺ staining with filamentous or missing ThioS⁺ labeling) compared to 5×FAD mice (FIGS. 4H-4J), a pattern that is opposite of that found in the Trem2-deficient mice crossed to 5×FAD (Yuan et al., 2016, Neuron, 90:724-739). Together, this study demonstrates that augmenting TREM2 expression can ameliorate Aβ pathology in 5×FAD mice.

Impact of Increased TREM2 Gene Dosage on Age-Dependent Transcriptional Profiles in 5×FAD Mice

To evaluate the impact of increasing TREM2 gene dosage on the molecular pathogenesis in 5×FAD mice, transcriptional profiling of the cortical samples from WT, BAC-TREM2, 5×FAD, and 5×FAD/TREM2 mice was performed at 2, 4, and 7 months of age. First, the transcripts specifically mapping to murine Trem2 or human TREM2 (FIGS. 2A and 2B) were examined. A low baseline level of murine Trem2 reads was observed that are significantly increased in 5×FAD and 5×FAD/TREM2 cortices at an advanced (7 months) but not at early (4 months) disease stage. Interestingly, the human TREM2 reads in 5×FAD/TREM2 mice significantly increased compared to those in BAC-TREM2 mice at 4 and 7 months, but not at 2 months. Together, the results suggest the BAC-TREM2 transgene drives early low level overexpression of human TREM2 in 5×FAD mouse brains, and there is a surge in the TREM2 transgene expression as disease progresses, recapitulating the disease-associated upregulation of murine Trem2 in this model.

Next, principal component (PC) analyses including all the RNA-seq samples at the three ages (FIGS. 6A-6C) was performed. A more defined separation was observed, based on 5×FAD genotypes at 4 and 7 months only, but a clear separation based on BAC-TREM2 genotype was not detected. These findings suggest the impact of the transgene on gene expression in WT or 5×FAD background is not robust enough to be detectable with this analysis.

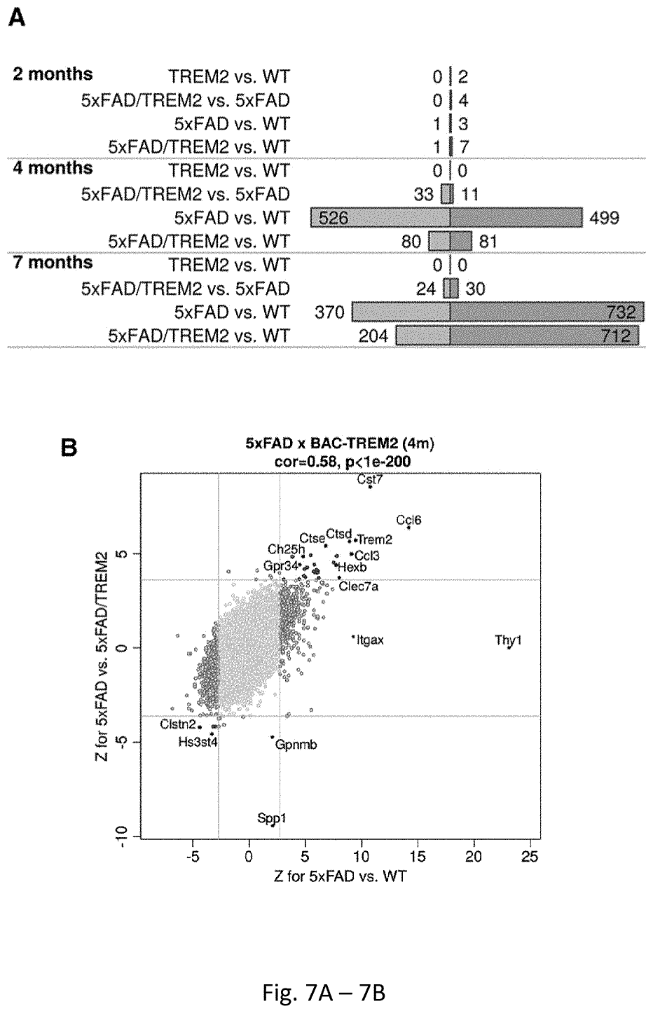

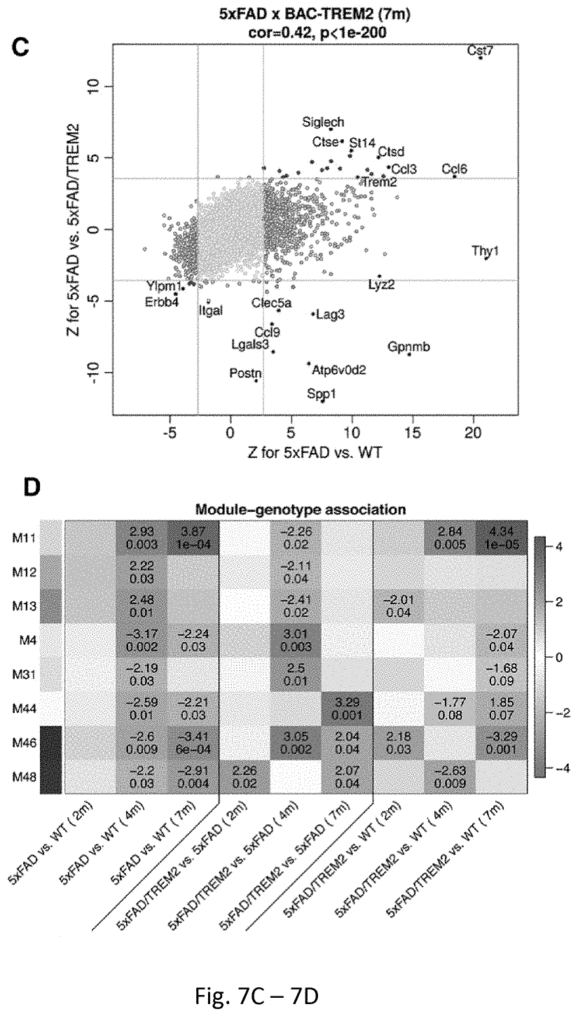

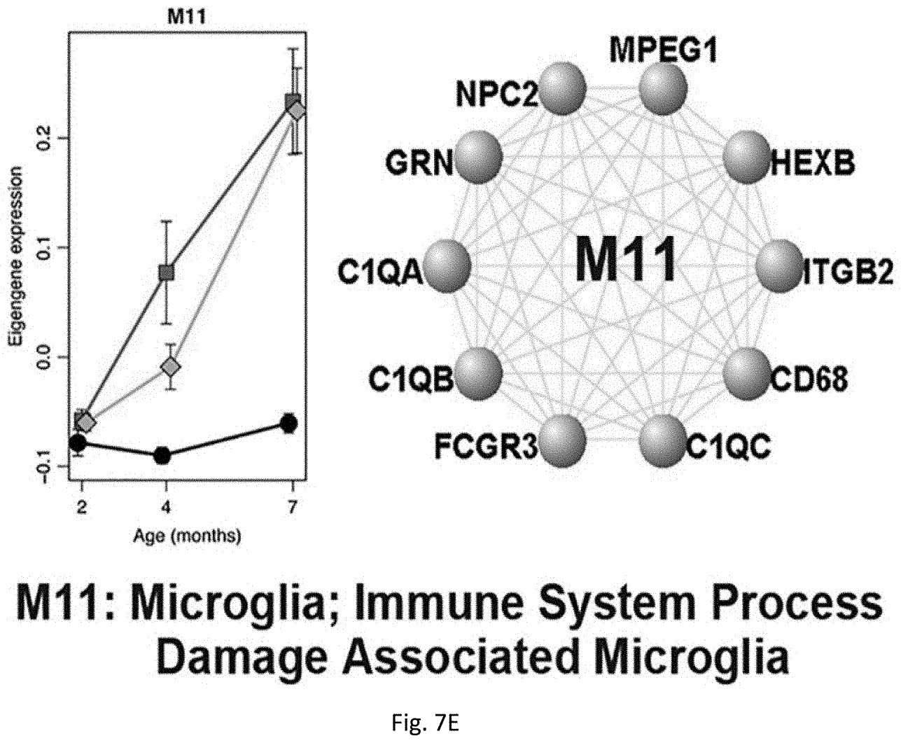

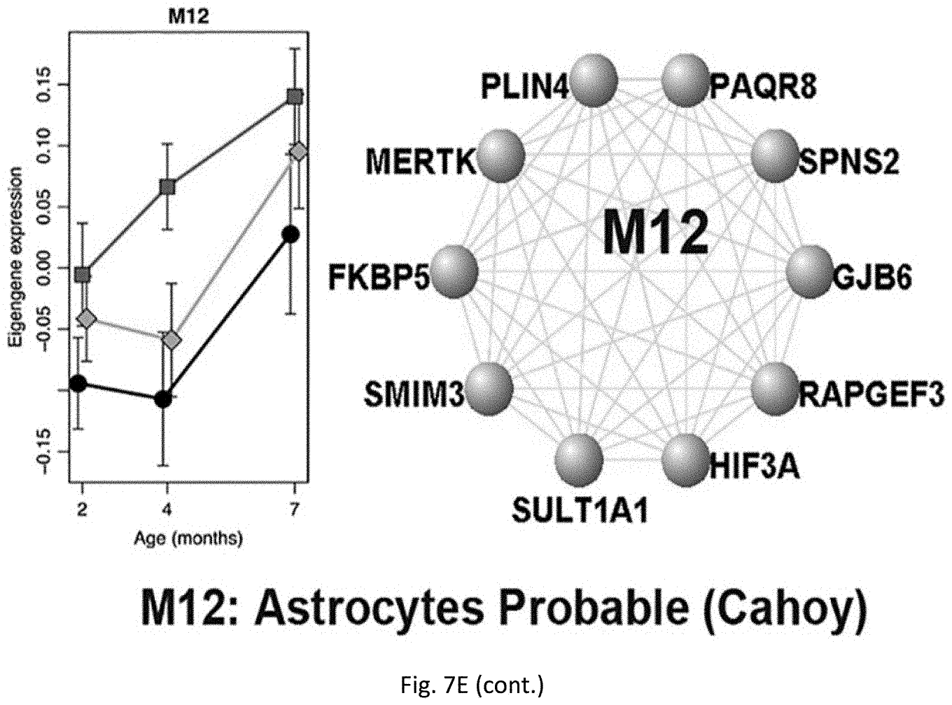

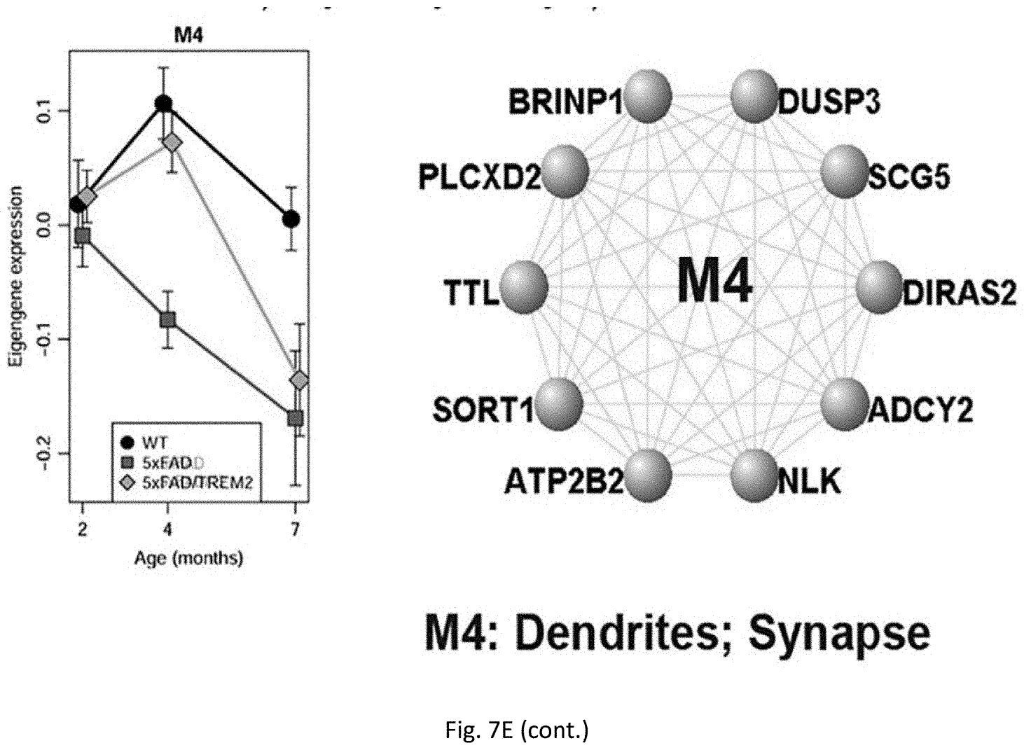

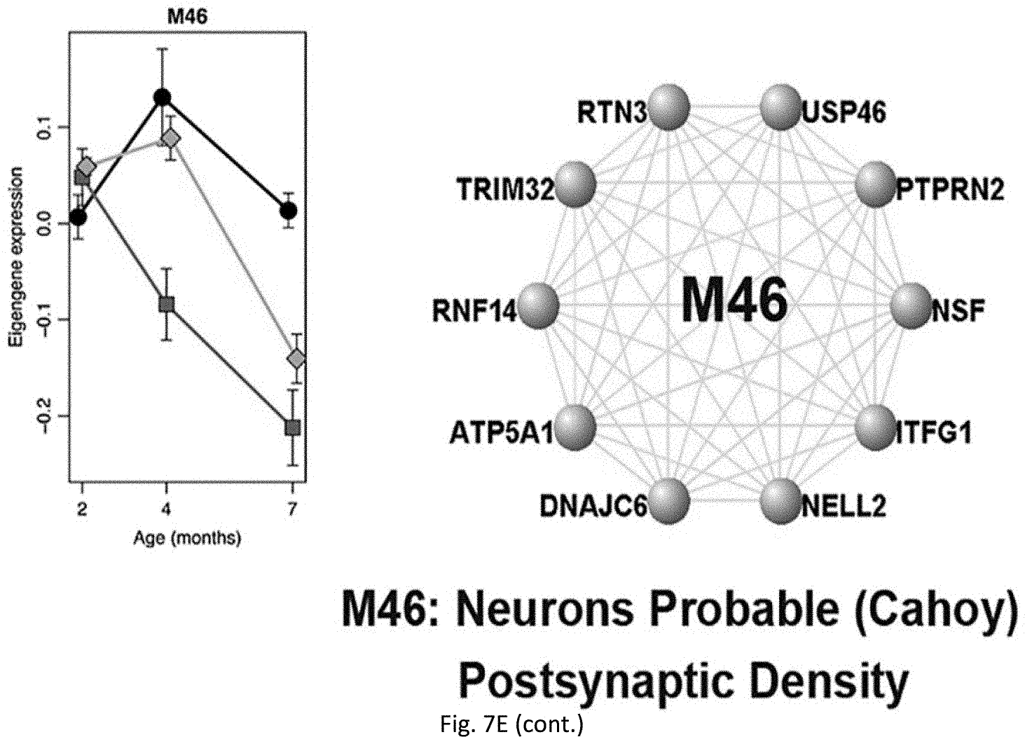

Genes with significant differential expression (DE) in the cortex were then examined among different genotypes across all three ages. As shown in FIG. 7A, very few genes were significantly differentially expressed (false discovery rate [FDR]<0.1) between BAC-TREM2 and WT at all ages, suggesting that the BAC-TREM2 transgene does not elicit a detectable molecular effect in the normal mouse brains. In 5×FAD mice, over 1,000 DE genes were observed at 4 and 7 months, but not at 2 months, consistent with the progression of cortical pathology in this aggressive amyloidosis model of AD (Oakley et al., 2006, J. Neurosci., 26:10129-10140). Interestingly, 5×FAD/TREM2 mice had only 161 DE genes at 4 months, and 916 genes at 7 months compared to WT, both of which are fewer than those in 5×FAD littermates. Finally, 5×FAD/TREM2 mice had 44 and 54 DE genes when compared to 5×FAD mice at 4 and 7 months of age, respectively.

Enrichment analyses of DE genes was performed using both public gene sets (e.g., GO and MSigDB) and an internal gene set collection (Langfelder et al., 2016, Nat. Neurosci., 19:623-633). Consistent with the disease process in 5×FAD mice and AD patients, the top enrichment terms for downregulated genes in 5×FAD versus WT were “neurons” (p=7.07E−55) and “synapses” (p=6.95E−19) at both 4 and 7 months, and the top enrichment for upregulated genes in 5×FAD mice were “top microglia genes” (p=3.19E−194) and “immune system process” (p=9.51E−83). The few DE genes between 5×FAD/TREM2 and 5×FAD were highly enriched in microglia and immune cell activation annotations (Galatro et al., 2017, Nat. Neurosci., 20:1162-1171).

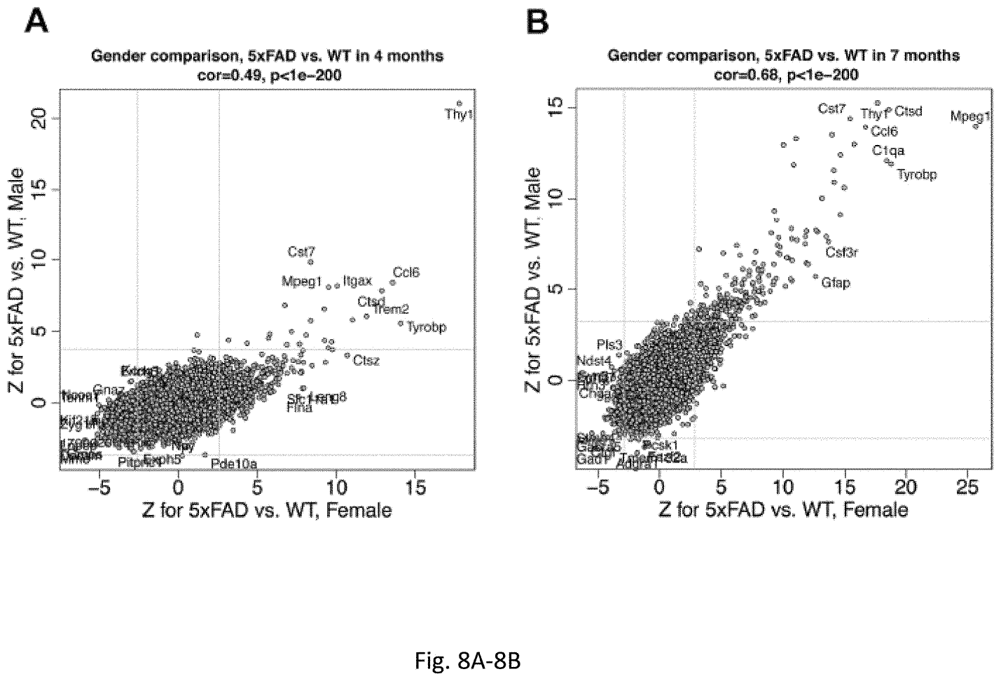

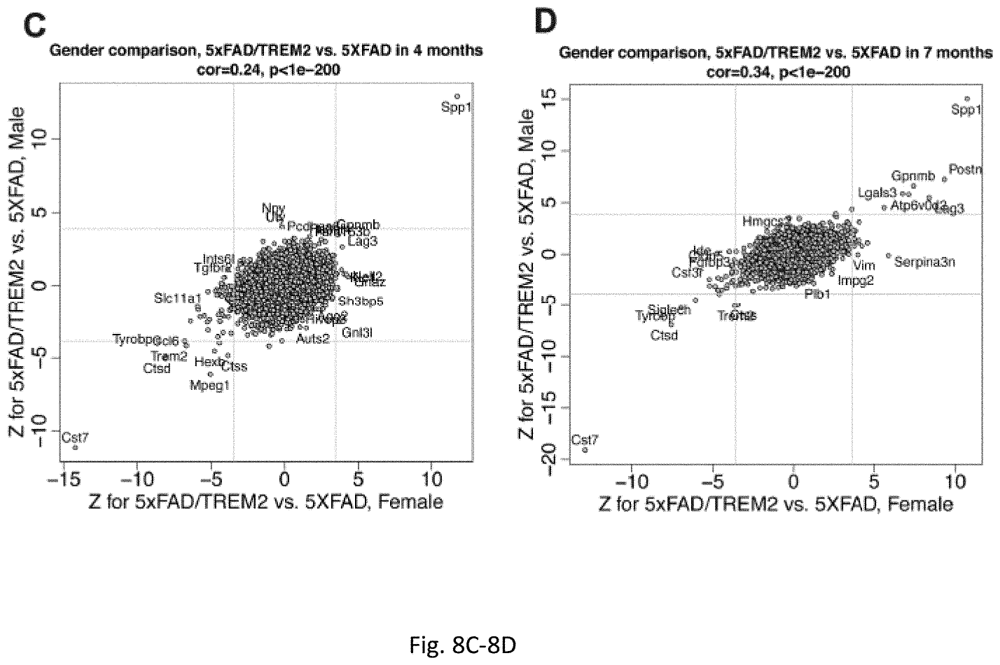

Prior studies of APP transgenic models including 5×FAD showed earlier and more severe disease-related phenotypes such as amyloid plaque pathology in female compared to male mice (Oakley et al., 2006, J. Neurosci., 26:10129-10140, Sadleir et al., 2015, Mol. Neurodegener., 10:1), which is reminiscent of sex differences in AD (Mazure and Swendsen, 2016, Lancet Neurol., 15:451-452). However, sex differences in the transcriptomic effects in 5×FAD mouse brains have not been reported. In the RNA-seq study, notwithstanding the limited sample size (n=3 per sex/genotype/age group; except n=2 for 5×FAD females at 7 months), more DE genes were observed in female than in male 5×FAD mice at both 4 and 7 months (FIGS. 6D-6I). Similarly, more DE genes were detected comparing 5×FAD/TREM2 to 5×FAD in female than male transgenic mice at these ages. These findings are consistent with the earlier and more severe cortical pathology in female compared to male 5×FAD mice (Oakley et al., 2006, J. Neurosci., 26:10129-10140, Sadleir et al., 2015, Mol. Neurodegener., 10:1). The correlation between DE statistics observed in male versus female animals when comparing (1) 5×FAD versus WT and (2) 5×FAD/TREM2 versus 5×FAD animals, at both 4 and 7 months (FIG. 8) was examined. This correlation between fold changes is positive and highly significant in all cases, supporting the notion that the two sexes behave similarly in all comparisons. Hence, although female 5×FAD mice have more significant DE genes when compared to WT or 5×FAD/TREM2, the transcriptional changes in the male mice are trending in the same direction. This is further supported by formal statistical testing for interactions of genotype and sex, which showed only few sex-dependent, significant DE genes across genotypes (FIGS. 6D-6F). Thus, it was concluded that transcriptomic changes occur earlier and more robustly in female than male 5×FAD mice, and that the effect of increased TREM2 gene dosage is also more apparent in female than male 5×FAD mice. However, without being bound by theory, but because of the strong correlation of fold changes between male and female mice in all comparisons, and the lack of strong statistical genotype/sex interaction effects, it was reasoned that a combined analysis of both sexes is a reasonable approach to identify the most consistent DE genes in 5×FAD, and those that are modulated by BAC-TREM2 in 5×FAD.

Partial Rescue of Transcriptional Dysregulation by Increasing TREM2 Gene Dosage in 5×FAD/TREM2 Mice

Next it was investigated whether increased TREM2 gene dosage in 5×FAD/TREM2 mice leads to overall rescuing or exacerbating effects on the transcriptional dysregulation observed in 5×FAD mice (compared to WT). The Z statistics for 5×FAD versus 5×FAD/TREM2 were compared against those for 5×FAD versus WT in a transcriptome-wide “rescue/exacerbation plot” (FIGS. 7B and 7C). A positive correlation can be interpreted as an overall transcriptome-wide “rescue effect,” and, conversely, a negative correlation would indicate an exacerbation effect. In both 4- and 7-month-old mice, the plots show a highly significant positive correlation (FIGS. 7B and 7C), demonstrating an overall transcriptome-wide rescuing effect in 5×FAD/TREM2 mice.

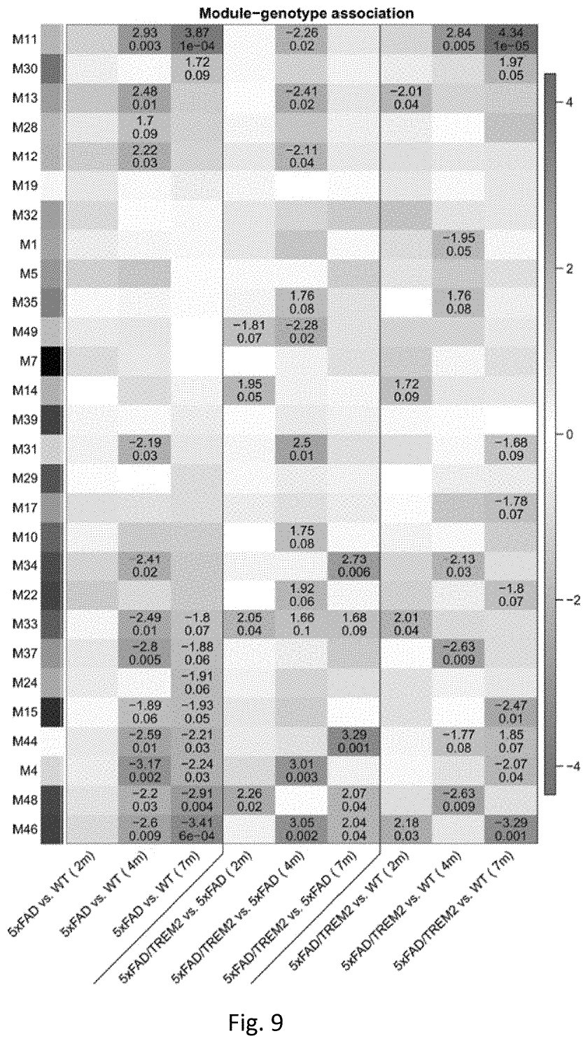

To further identify molecular networks that may be selectively targeted by increased TREM2 gene dosage in 5×FAD/TREM2 mouse brains, consensus weighted gene co-expression network analyses (WGCNA; Langfelder and Horvath, 2008, BMC Bioinformatics, 9:559) was performed. The WGCNA approach has been used previously to identify coherent gene modules dysregulated in AD mouse and patient brains (Matarin et al., 2015, Cell Rep., 10:633-644; Miller et al., 2008, J. Neurosci., 28:1410-1420). The consensus WGCNA analysis identified 28 co-expression modules (FIG. 9). By relating the eigengene (a single representative expression profile of a module, Horvath and Dong, 2008, PLoS Comput. Biol., 4:e1000117) to genotypes, five modules (M11, M12, M13, M28, and M30) were significantly (p<0.05) upregulated in 5×FAD versus WT mice at 4 or 7 months, and 9 modules (M4, M15, M24, M31, M33, M37, M44, M46, and M48) were downregulated in 5×FAD mice. Importantly, three of the five upregulated 5×FAD modules (M11, M12, and M13) and five of the nine downregulated AD modules (M4, M31, M44, M46, and M48) are partially rescued in 5×FAD/BAC-TREM2 mice (FIG. 7D).

Enrichment analyses of these network modules revealed that two of the three modules that were upregulated in 5×FAD and partially rescued in 5×FAD/TREM2 were annotated for microglia-enriched (M11) and astrocyte-enriched genes (M12). M11 is a large module (1,232 genes) enriched with terms such as “immune system processes” and “damage-associated microglia” (Keren-Shaul et al, 2017, Cell, 169:1276-1290) (FIG. 7E). Top hub genes in M11 module are known to be involved in microglial function (e.g., Grn and C1q). The M11 eigengene was progressively upregulated in 5×FAD compared to WT mice. This elevation was significantly reduced at 4 months in the 5×FAD/TREM2 mice (FIGS. 7D and 7E). M12, a smaller module (119 genes), was enriched with astrocyte genes. The upregulation of genes in M12 was also diminished in 5×FAD/TREM2 compared to 5×FAD mice (FIGS. 7D and 7E). One of the M12 hub genes is Mertk, a phagocytic receptor involved in astrocyte- and microglia-mediated phagocytosis of synapses and neurons (Brown and Neher, 2014, Nat. Rev. Neurosci., 15:209-216, Chung et al., 2015, Nat. Neurosci., 18:1539-1545).

Among the five modules that have decreased expression in the 5×FAD mice and are partially rescued in 5×FAD/TREM2 mice, two are particularly interesting. M4 is significantly enriched in terms including “dendrite” and “synaptic genes” (e.g., Atp2b2, Grik2, Grin1, Mapt), and M46 (347 genes) is enriched in “neuronal genes” and “genes downregulated in the hippocampi of AD patients” (FIGS. 7D and 7E). Increased TREM2 gene dosage in 5×FAD/TREM2 mice significantly improved these modules at 4 months, and for M46 such effect appeared to be extended to 7 months (FIG. 7E). Thus, increased TREM2 expression in microglia may exert non-cell-autonomous effects to partially restore neuronal gene expression in the 5×FAD mice.

Reprogramming Disease-Associated Microglia Gene Expression Signatures in 5×FAD Mice by TREM2 Gene-Dosage Increase

Systems biology has played a powerful role in the unbiased discovery of microglia function and dysfunction in the brain (Galatro et al., 2017, Nat. Neurosci., 20:1162-1171, Gosselin et al., 2017, Science, 356:eaal3222, Zhang et al., 2013, Cell, 153:707-720). In 5×FAD mice, Trem2 deficiency greatly impaired the overall transcriptional response of reactive microglia (Wang et al., 2015, Cell, 160:1061-1071). Two recent studies examined microglial molecular signatures that are associated with disease progression in mouse models of neurodegenerative disorders, including AD and amyotrophic lateral sclerosis (ALS) (Keren-Shaul et al, 2017, Cell, 169:1276-1290; Krasemann et al., 2017, Immunity, 47:566-581). These studies identified overlapping microglial gene sets termed damage-associated microglia (DAM) genes (Keren-Shaul et al, 2017, Cell, 169:1276-1290) or molecular signatures of disease-associated microglia (MGnD) (Krasemann et al., 2017, Immunity, 47:566-581). Importantly, both studies showed a critical set of microglial genes that are dependent on Trem2 for their disease-associated upregulation. In this study, the effects of increased TREM2 gene dosage on these known disease-associated microglial molecular signatures in the AD mice was investigated.

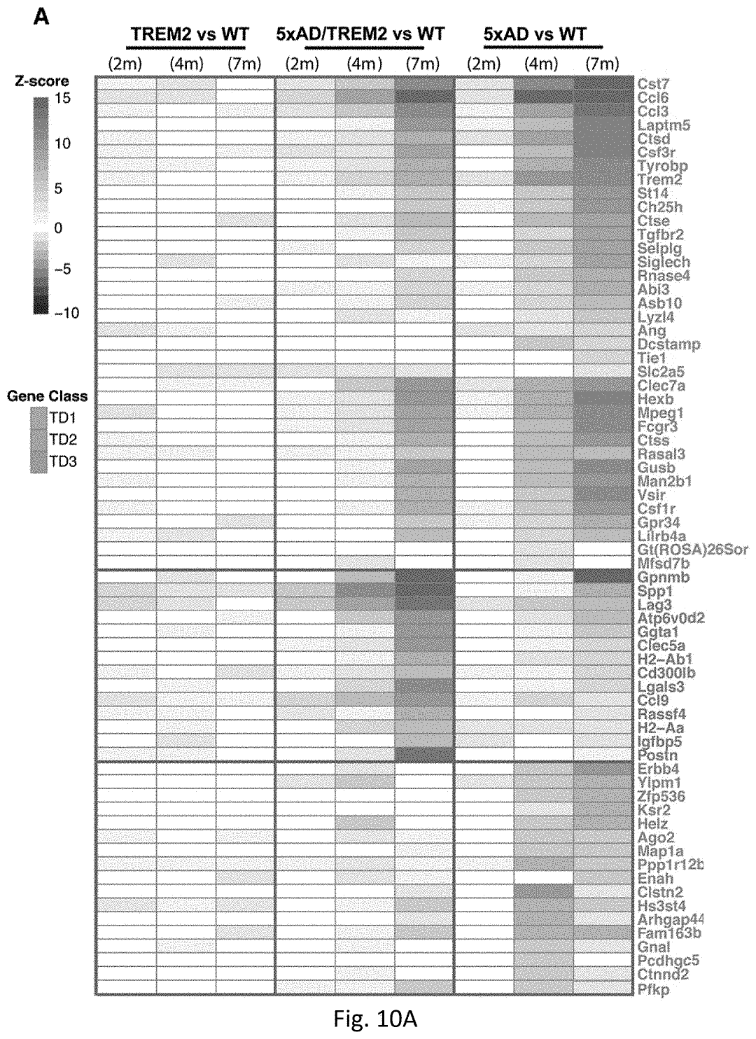

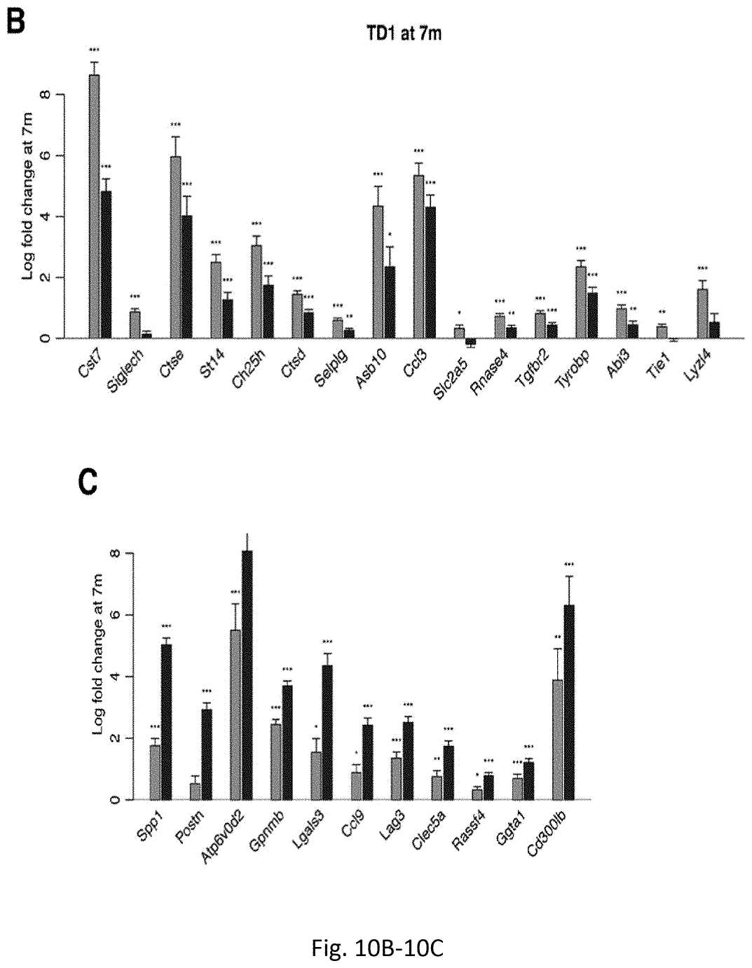

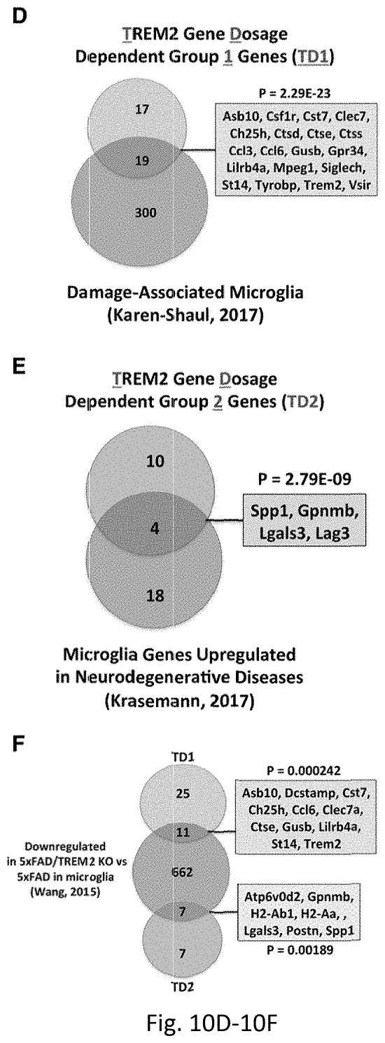

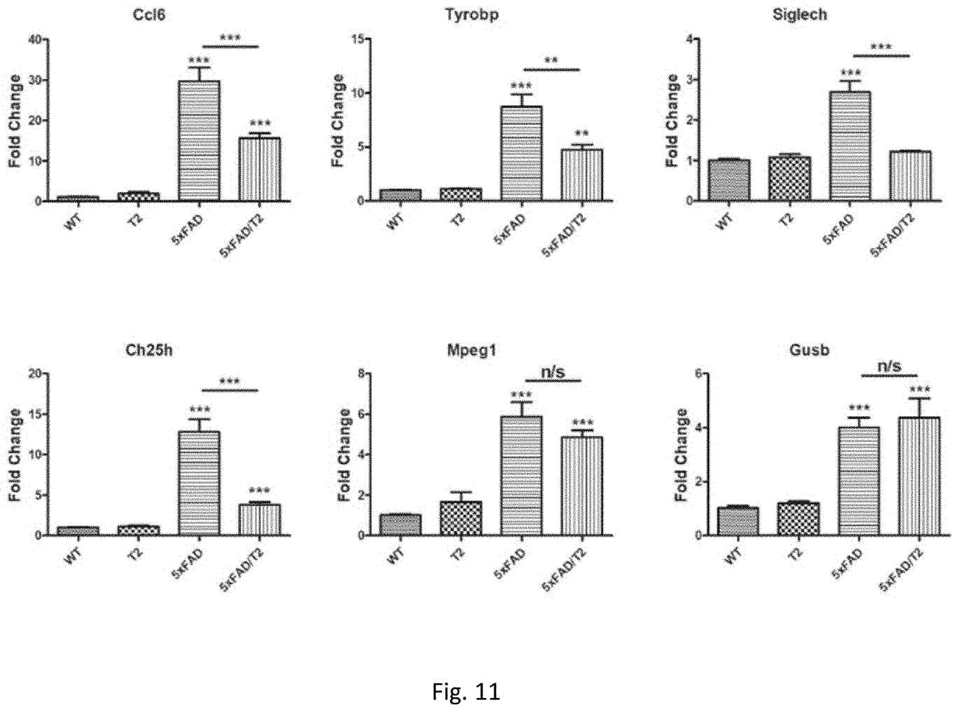

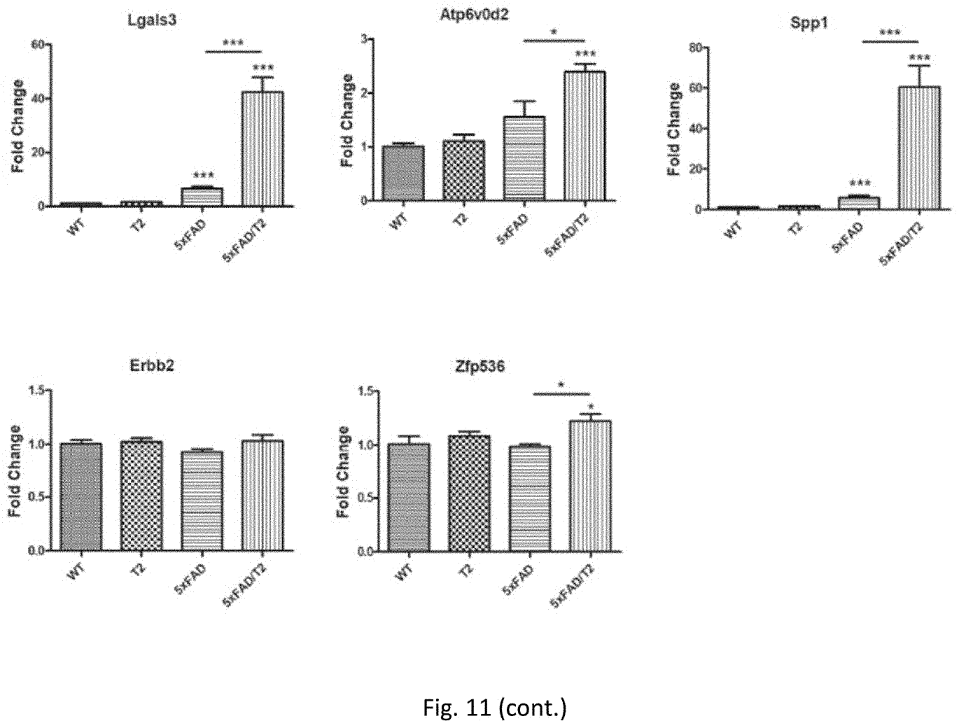

Based on the transcriptome-wide rescue/exacerbation plots (FIGS. 7B and 7C), three sub-groups of DE genes were defined between 5×FAD/TREM2 and 5×FAD (termed TD1-TD3, for TREM2 dosage-dependent genes; FIG. 10A). First, DE genes between 5×FAD/TREM2 and 5×FAD (FDR<0.1) at either 4 or 7 months were selected. Among these genes, TD1 genes are those with expression levels significantly upregulated in 5×FAD compared to WT (Z>2), but are significantly downregulated in 5×FAD/TREM2 compared to 5×FAD (Z<−3). The TD1 genes are those located in the upper-right quadrant of the plots (FIGS. 7B and 7C). TD2 genes are those significantly upregulated in 5×FAD mice versus WT (Z>2) and further upregulated in 5×FAD/TREM2 versus 5×FAD (Z>3). These are genes located in the lower-right quadrant of the plots (FIGS. 7B and 7C). Finally, TD3 genes are in the lower-left quadrant of the rescue/exacerbation plots and are significantly downregulated in 5×FAD versus WT (Z<−2) but were significantly upregulated in 5×FAD/TREM2 versus 5×FAD (Z>3). No transcripts were observed that are downregulated in 5×FAD mice and are further decreased in 5×FAD/TREM2 mice. Importantly, real-time RT-PCR was applied to validate a critical subset of TD1-TD3 genes for their appropriate DE profiles in 5×FAD and 5×FAD/TREM2 cortices (FIG. 11).

Next enrichment analyses of TD1-3 was performed. The top enrichment terms for TD1 genes are “Upregulated in damage associated microglia” (p=2.29E−23; Keren-Shaul et al, 2017, Cell, 169:1276-1290), “top human microglia-specific genes” (p=2.15E−16; Galatro et al., 2017, Nat. Neurosci., 20:1162-1171), “Autophagy lysosome” (p=4.90E−09), “genes upregulated with age in hippocampus” (p=2.89E−08), and “immune system process” (p=0.000142). These annotations suggest TD1 genes are key microglial genes normally involved in microglia activation in diseased brain and are significantly reversed with increased TREM2 gene dosage. Despite the relatively small number of genes in TD1 group, they were among the top upregulated, DAM genes in 5×FAD mice (based on Z statistics; FIGS. 7B, 7C, and 10D). The 19 DAM genes (Keren-Shaul et al, 2017, Cell, 169:1276-1290; FIG. 10D) that are partially, but significantly, rescued in 5×FAD/TREM2 mice, including Cst7 (the top upregulated microglial gene in 5×FAD brain), several cathepsins (Ctsd, Ctse, Ctss), chemokines (Ccl3, Ccl6) and their receptors (Csf1r), and established AD-associated genes (Trem2 and Abi3) (Sims et al., 2017, Nat. Genet., 49:1373-1384).

The TD2 genes constitute possibly the most interesting group, despite its relatively small number (14 genes). The top enrichment term in this group is “Microglia genes upregulated in neurodegenerative diseases” (p=2.79E−09; Krasemann et al., 2017, Immunity, 47:566-581). Four genes belonging to this group (Spp1, Gpnmb, Lgals3, and Lag3) were significantly further upregulated in 5×FAD/TREM2 versus 5×FAD mice at 4 and 7 months (FIGS. 10A and 10E). Importantly, the other top enrichment terms for TD2 genes were GO terms such as “Negative regulation of T cell activation” (p=4.96E−09) and “Innate immune response” (p=8.87E−07). TD2 genes were well characterized for regulation of phagocytosis and microglial activation (e.g., Lgals3; Rotshenker, 2009, J. Mol. Neurosci., 39:99-103), microglial survival (e.g., Spp1; Rabenstein et al., 2016, J. Neuroimmunol., 299:130-138), alternative M2 activation of microglia (Postn; Zhou et al., 2015, Nat. Cell Biol., 17:170-182), and lysosomal proton pump (Atp6v0d2). These analyses suggest increasing TREM2 gene dosage selectively upregulates an interesting subset of “disease-associated microglial genes” to promote certain aspects of microglial function, such as phagocytosis and suppression of over-activation of the innate immune response in the AD mouse brain.

Next, it was asked what genes altered in 5×FAD/TREM2 mice overlap with those altered in 5×FAD/Trem2^{-/-} (Wang et al., 2015, Cell, 160:1061-1071). A total of 11 TD1 and 7 TD2 genes overlapping with the downregulated genes in 5×FAD/Trem2^{-/-}microglia (FIG. 10F) were identified. Of these genes, the seven TD2 genes are potentially interesting, as they were downregulated in 5×FAD mice in the absence of Trem2 but upregulated in 5×FAD mice with increased TREM2 gene dosage. They may constitute transcriptional targets of Trem2 gene-dosage-dependent signaling in the context of disease-associated microglia.

Finally, the TD3 group is only modestly enriched for postsynaptic density genes, consistent with the results of network analyses showing that a subset of neuronal and synaptic genes downregulated in 5×FAD mice were partially normalized with TREM2 overexpression.

Increased TREM2 Gene Dosage Alters Microglial Interaction with Amyloid Plaques

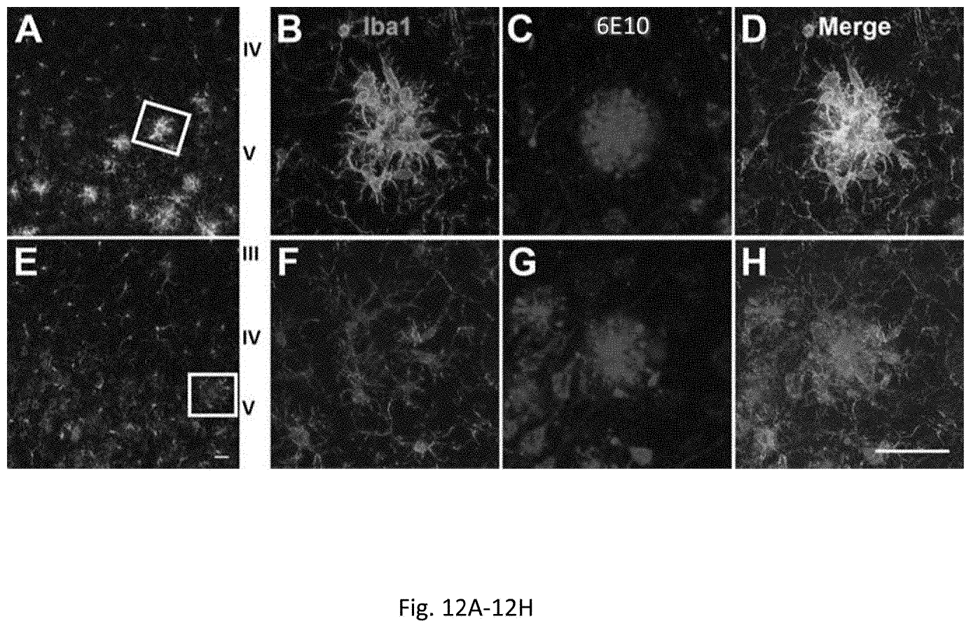

A primary feature of reactive microgliosis is morphological transformation (Stence et al., 2001, Glia, 33:256-266). In 5×FAD mice, plaque-associated microglia exhibited shortened and thickened processes with hypertrophic amoeboid shape, representing canonical reactive microglia (FIGS. 12A-12D). However, these phenotypes were markedly attenuated in 5×FAD/TREM2 mice (FIGS. 12E-12H). Unlike the Trem2-deficient microglia in AD mice (Jay et al., 2015, J. Exp. Med., 212:287-295; Wang et al., 2015, Cell, 160:1061-1071), the plaque-associated microglia in 5×FAD/TREM2 mice show more elongated and ramified processes than those in the 5×FAD mice (FIGS. 12E-12H). Without being bound by theory, these findings were interpreted as evidence for reprogrammed microglial response in 5×FAD/TREM2 mice, so that morphologically less activated (i.e. more ramified) microglia surrounding the plaque are still able to function effectively in limiting the size and diffusion of the amyloid plaque in 5×FAD cortices.

Increased TREM2 Gene Dosage Enhances Phagocytic Microglia Markers In Vivo and Phagocytic Activity In Vitro

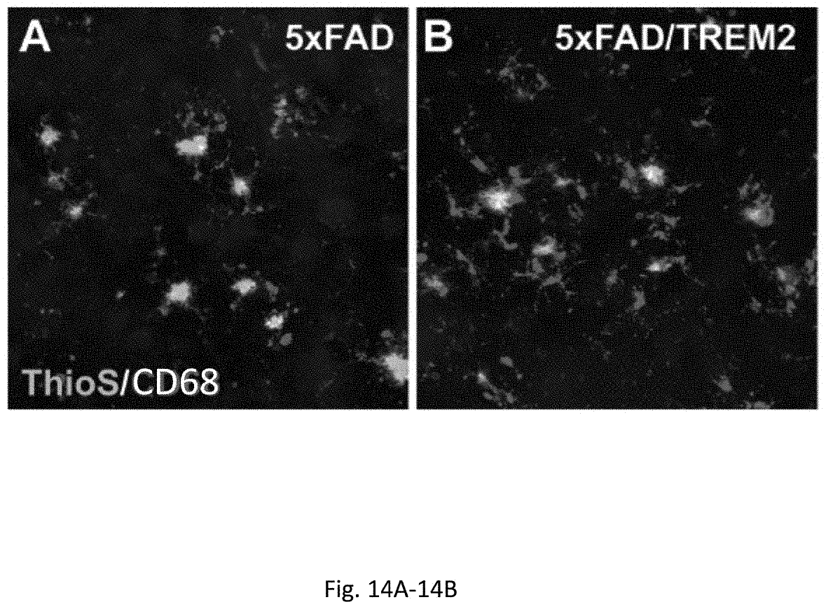

Based on the reduced amyloid burden and the upregulation of several phagocytosis-related TD2 genes in the 5×FAD/TREM2 mice, without being bound by theory, it was hypothesized that phagocytic activity of microglia might be enhanced by increased TREM2gene dosage in AD mouse brains. CD68 protein expression, a marker for phagocytosis shown to be upregulated in plaque-associated microglia in AD brains (Yuan et al., 2016, Neuron, 90:724-739) was assessed. A significant increase of anti-CD68 staining was found in the plaque-associated microglia in 5×FAD/TREM2 cortices compared to those in 5×FAD cortices at 7 months of age (FIGS. 13A-13C and 14). This result suggests that a post-transcriptional mechanism may be involved in the upregulation of CD68 in 5×FAD/TREM2 microglia surrounding the plaque, as transcript amounts are comparable between 5×FAD and 5×FAD/TREM2 at this age. Further, the level of a second microglial phagocytosis related protein, Lgals3 (Rotshenker, 2009, J. Mol. Neurosci., 39:99-103), which is one of the TD2 genes was assessed. Very little staining for Lgals3 was detected in WT mouse brains at this age. In the 5×FAD/TREM2 mice, a significant increase in the number of Lgals3⁺ microglia surrounding the amyloid plaque was observed compared to the 5×FAD mice (FIGS. 13F and 13D); hence supporting the concept that upregulation of phagocytic markers is part of the reprogrammed microglia response in 5×FAD/TREM2 mice.

To functionally evaluate the phagocytic capacity of microglia in the models, at least in vitro, a phagocytosis assay was performed with primary microglia isolated from neonatal mice. Such an in vitro assay provided a model to evaluate the stress-induced phagocytic activity in the microglia (Gosselin et al., 2017, Science, 356:eaal3222). Polystyrene microbeads were used to assess the general phagocytic activity, and also to ensure phagocytosis rather than pinocytosis was being measured. The results showed a significant enhancement of phagocytic activity in BAC-TREM2 microglia compared to WT microglia, while Trem2^{-/-}microglia showed a significant decrease in phagocytic activity (FIG. 13G). Importantly, such a deficit in Trem2-deficient microglia can be rescued by the BAC-TREM2 transgene. These results suggest that Trem2 level is a rate-limiting factor in regulating the phagocytic activities of microglia in vitro. Furthermore, the rescue assay showed that Trem2 function in regulating microglia phagocytosis is preserved and elevated in the BAC-TREM2 transgene. In summary, these studies showed that enhanced phagocytic activity could be another key component of reprogrammed microglial function by BAC-TREM2 in AD mouse brains.

Upregulation of TREM2 in Microglia Ameliorates Cognitive Deficit

Previous studies reported the presence of neurite dystrophy in close association with Aβ deposits in AD patients and mouse models (Masliah et al., 1996, J. Neurosci., 16:5795-5811; Nixon, 2007, J. Cell Sci., 120:4081-4091), and this phenotype is exacerbated with AD risk-associated TREM2 variants (Wang et al., 2016, J. Exp. Med., 213:667-675; Yuan et al., 2016, Neuron, 90:724-739). The transcriptomic analyses suggest that the downregulated neuronal genes may partially be recovered in the 5×FAD/TREM2 mice.

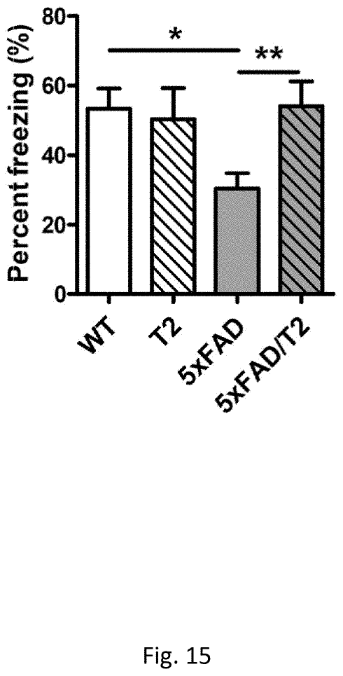

To evaluate whether the neuroprotective phenotypes observed in 5×FAD/TREM2 mice may correspond to a behavioral improvement, the contextual fear-conditioning test, a hippocampus-dependent memory task that is compromised in 5×FAD mice (Kimura and Ohno, 2009, Neurobiol. Dis., 33:229-235) was performed. Impressively, unlike the 5×FAD mice that exhibited a robust deficit in this task, the performance of 5×FAD/TREM2 mice is comparable to that of WT controls (FIG. 15). Moreover, BAC-TREM2 mice were not significantly different from WT mice, suggesting the BAC-TREM2 transgene alone does not affect this memory task. Thus, it was concluded that the BAC-TREM2 transgene is improving both the neuritic pathology and cognitive performance in an amyloid mouse model of AD.

Increased TREM2 Gene Dosage Alters Plaque-Associated Microglia Morphology and Ameliorates Behavioral Deficit in a Second Mouse Model of AD

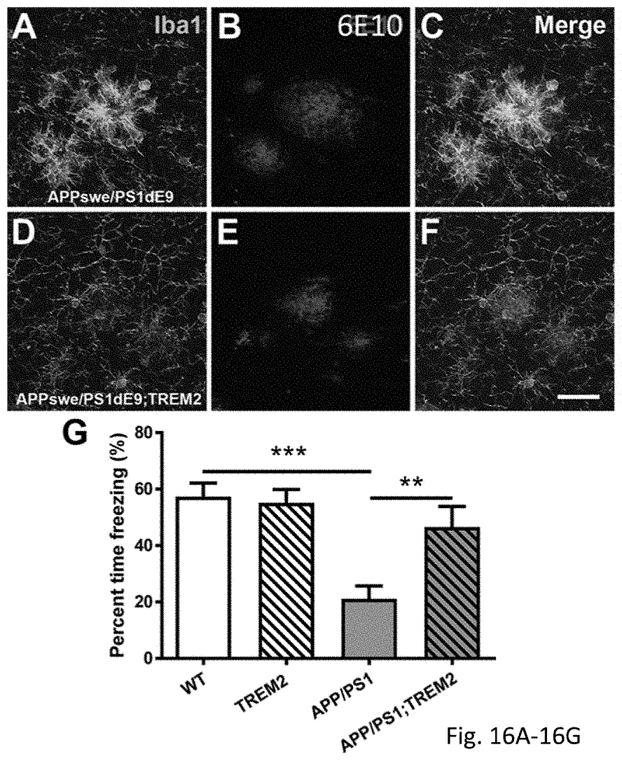

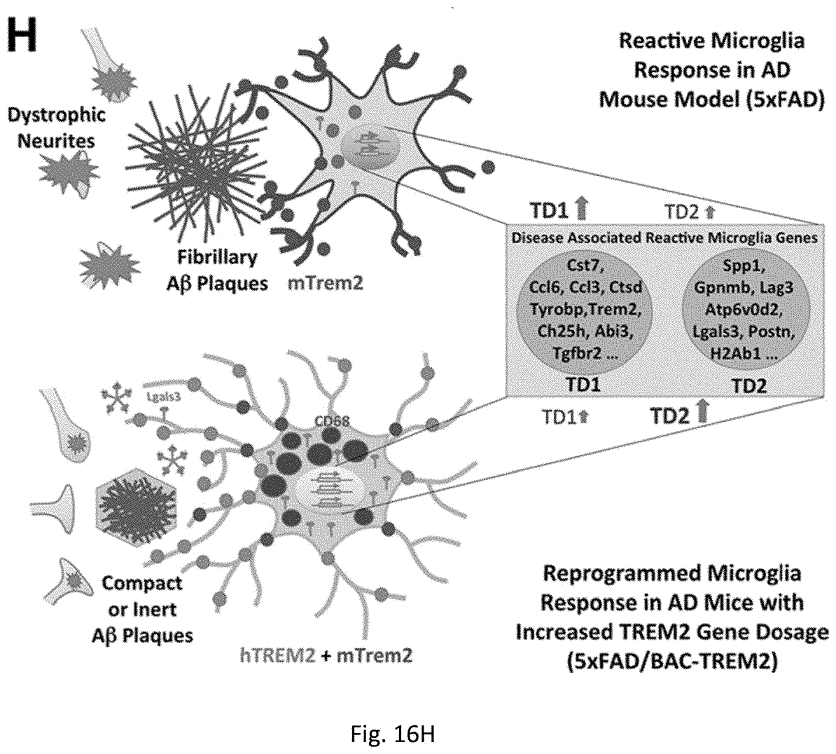

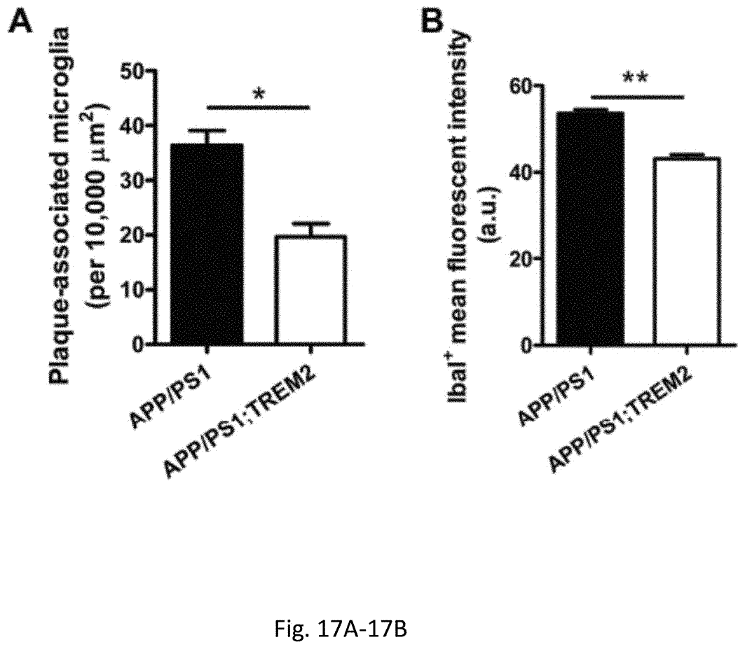

To validate and extend some of the findings in 5×FAD/TREM2 mice in another amyloid mouse model of AD, BAC-TREM2 mice were crossed with APPswe/PS1dE9 (APP/PS1) mice, another commonly used mouse model of AD (Jankowsky et al., 2004, Hum. Mol. Genet., 13:159-170). This model has slower onset of amyloid pathology that affects hippocampus as well as the cortex. At 11 months of age, a marked alteration of reactive microglia morphology surrounding the plaques (i.e., more elongated processes) in the hippocampus of the APP/PS1; BAC-TREM2 (APP/PS1; TREM2) mice (FIGS. 16A-16F) was observed. There was also marked reduction of Iba1 immunostaining in the double transgenic mice compared to APP/PS1 mice, suggesting altered microglial reactivity (FIGS. 16A-16F and FIG. 17).

Similar to 5×FAD mice, APP/PS1 mice have been reported to show deficits in contextual fear conditioning (Knafo et al., 2009, J. Pathol., 219:41-51). Here, it was observed that, at 11 months of age, APP/PS1 mice exhibited a robust contextual fear conditioning deficit compared to WT mice, and this phenotype was abolished in the APP/PS1; TREM2 mice (FIG. 16G). The results suggest that microglial expression of the BAC-TREM2 transgene reprograms microglial responsivity and ameliorates the disease-associated behavioral impairment in a second AD mouse model.

### Example 2

### Engineered Mammalian Animal and Cell Models to Enable the Identification of Molecules that Target Microglia and Myeloid Cell Function

One emerging theme in AD GWAS studies are that the genes significantly modifying the risk of late-onset AD seem to have known function in microglia, and many of them are selectively expressed in the microglia in the brain (Hansen et al., 2018, J Cell Biol, 217:459-472; Efthymiou and Goate, 2017, Mol Neurodegener, 12:43; Wes et al., 2016, Glia, 64:1710-1732). There are about two dozen AD GWAS significant genes with relatively enriched expression in microglia compared to many other brain cell types in human and mouse brains, i.e. ABCA7, PTK2B, RIN3, SORL1, SPI1, ZCWPW1, CR1, NME8, CD33, MS4A4A, MS4A6A, MS4A4E, MS4A6E, MS4A2, IL1RAP, INPP5D, PLCG2, PICALM, HLA-DRB1, TREM2, TREM1, TREML1, ABI3, CASS4, CD2AP, EPHA1, MEF2C (Hansen et al., 2018, J Cell Biol, 217:459-472; Efthymiou and Goate, 2017, Mol Neurodegener, 12:43; Wes et al., 2016, Glia, 64:1710-1732). Moreover, a number of these microglia-enriched, and AD-associated genes also have genetic, physical or functional interactions with TREM2, and appear to function in a broad TREM2 signaling pathway (Hansen et al., 2018, J Cell Biol, 217:459-472). For example, APOE (with its E4 variant being #1 AD GWAS genes in terms of effect size and prevalence in the population) and CLU (APOJ) directly binds to TREM2 (Yeh et al., 2017, Trends Mol. Med., 23:512-533). Genetic analyses reveal potential influence of CD33 on TREM2 expression (Chan et al., 2015, Nat Neurosci, 18:1556-1558), and a protective allele in AD is resided in PLCG2, which functions downstream of TREM2 signaling (Sims et al., 2017, Nat. Genet., 49:1373-1384; Efthymiou and Goate, 2017, Mol Neurodegener, 12:43). Moreover, common variants in the MS4A gene cluster (MS4A4A, MS4A6A, and MS4A4E) are shown to be genome-wide significantly associated with the soluble TREM2 (sTREM2) levels in the cerebrospinal fluid (CSF) of AD patients and are also significantly associated with TREM2 mRNA levels in multiple tissues (Demning et al, 2018, bioRxiv,352179). Furthermore, MS4A4A is shown in cell models to be involved in the trafficking of TREM2 to the cell membrane. Thus, converging evidence suggest the microglial-specific genes, particularly those revealed as GWAS significant in AD, could be key molecular targets to modify microglial function and to treat age-dependent neurodegenerative disorders including AD.

TREM2 Gene-Dosage Increase in Two AD Mouse Models

Human genetic studies suggest a large subset of GWAS significant SNPs are more likely to be expression quantitative trait locus (eQTLs), which modify the expression level of the adjacent genes and lead to modification of disease phenotypes (Nicolae et al., 2010, PLoS Genet, 6:e1000888). In AD, the GWAS alleles are shown to modify the expression of a subset of AD risk genes in the brain (Karch et al., 2014, Neuron 83:11-26) or in the peripheral monocytes (Chan et al., 2015, Nat Neurosci, 18:1556-1558). Hence, without being bound by theory, it is hypothesized that modulating the expression level of AD-associated, microglia-enriched genes at both RNA and protein levels and in appropriate cell types will modify microglial function and prevent or modify AD and potentially other brain diseases.

Analysis of the Direction of Expression Levels of Microglial-Expressed AD GWAS Genes that Could be Beneficial in Modifying AD or Other Disease Phenotypes

A key question is to which direction one should modulate the microglial enriched AD GWAS genes that could result in a therapeutic benefit in AD and other brain disorders. The answer to this question should be a combination of human studies (GWAS, eQTL, etc) and studies in animal models (expression, genetics). The examples of some answers to this question including the following:

TREM2 Upregulation:

It is suggested elsewhere herein that upregulation of TREM2 levels prior to the amyloid deposition and microglial activation could reprogram microglial responsivity, ameliorate neuronal injury and improve cognitive performance in AD mouse models. On the other hand, in several AD models and AD patient brains, loss of function of TREM2 or patient-associated TREM2 variants resulted in worsening of the barrier function of TREM2 and exacerbation of amyloid plaque load and neuronal toxicities (Wang et al., 2015, Cell, 160:1061-1071; Yuan et al., 2016, Neuron, 90:724-739; Wang et al., 2016, J. Exp. Med., 213:667-675; Xiang et al., 2018; Jay et al., 2017, J. Neurosci., 37:637-647; Cheng-Hathaway et al., 2018, Mol Neurodegener, 13:29). Hence, the directionality of TREM2 level regulation that could be therapeutically beneficial in AD and related brain disorders should be the upregulation in the microglia.

CD33 Down-Regulation:

The GWAS AD protective SNP (rs3865444A) for CD33 corresponds to lower expression level of CD33 at RNA and protein levels (Bradshaw et al., 2013, Nat Neurosci, 16:848-850; Schwarz et al, 2016, Proc Natl Acad Sci USA., 113:74-79) and reduction of amyloid beta 42 (Aβ42) levels in AD brain (Griciuc et al., 2013, Neuron, 78:631-643). Interestingly, this CD33 protective SNP variant is only found in human (with an allele frequency of 0.21), and not in other mammalian species (Schwarz et al, 2016, Proc Natl Acad Sci USA., 113:74-79). Moreover, CD33 impairs microglia uptake and clearance of Abeta 42, and deletion of murine CD33 reduces abeta pathology in the APPswe/PS1dE9 mouse model of AD (Griciuc et al., 2013, Neuron, 78:631-643). Thus, available evidence suggests downregulation of CD33 in microglia (and possibly other myeloid cells) could be beneficial in improving microglial function and reducing disease phenotypes in AD and other related brain disorders.

Developing Mammalian Cell and Animal Models to Study the Regulation of Microglial-Expressed AD GWAS Gene Expression Under Human Genomic Regulation.

Cell and animal models are developed that allow the discovery of molecules that can alter the expression levels of human ME-AD genes. The expression of such genes is evaluated under the human genomic regulatory context in cells and in vivo since all the mechanisms related to the expression of ME-AD genes, including but not limited to transcriptional regulation, RNA splicing, transport and metabolism, could have human-specific regulation and are preserved in human genomic DNA based reporters. There are many examples of human-specific context at DNA, RNA or protein levels that are important to human disease manifestation. For example, alpha Synuclein (aSyn) A53T is pathogenic variant for familial PD but normal variants in rodents. And interestingly, human but murine aSyn carrying the same variant is pathogenic in transgenic mice, suggesting the human aSyn context is critical for the neurotoxicity of aSyn variants (Sreeganga et al., 2005). There are several other examples of pathogenic variants in human are found as normal variants in other mammalian species (a phenomena termed cis-suppression of human disease mutations; Jordan et al., 2015, Nature, 524:225-229). A recent example demonstrates the ME-AD gene, TREM2, also show differential response in the human vs mouse gneomic context (Xiang et al., 2018, Mol Neurodegener, 13:49; Ma et al., 2016, Mol Neurodegener, 11:72; Cheng-Hathaway et al., 2018, Mol Neurodegener, 11:72). The pathogenic R47H variant impairs splicing and reduce mRNA levels only in the mouse but not human genomic context (ibid), underscoring the importance of studying human disease genes and their variants in the human genomic context. Together, these studies show the critical importance of the regulation of human ME-AD genes expressed from a genomic context of human genes.

Engineering Large Human Genomic Transgenic (HGT) DNA Constructs (e.g. BAC) to Drive the Expression of ME-AD Genes in Mammalian Animal and Cell Models.

The advantage of engineering large HGT is that is can be used to develop both cell models and intact mammalian animal models that express ME-AD reporters that are in endogenous-like patterns and driven by human genomic regulatory elements.

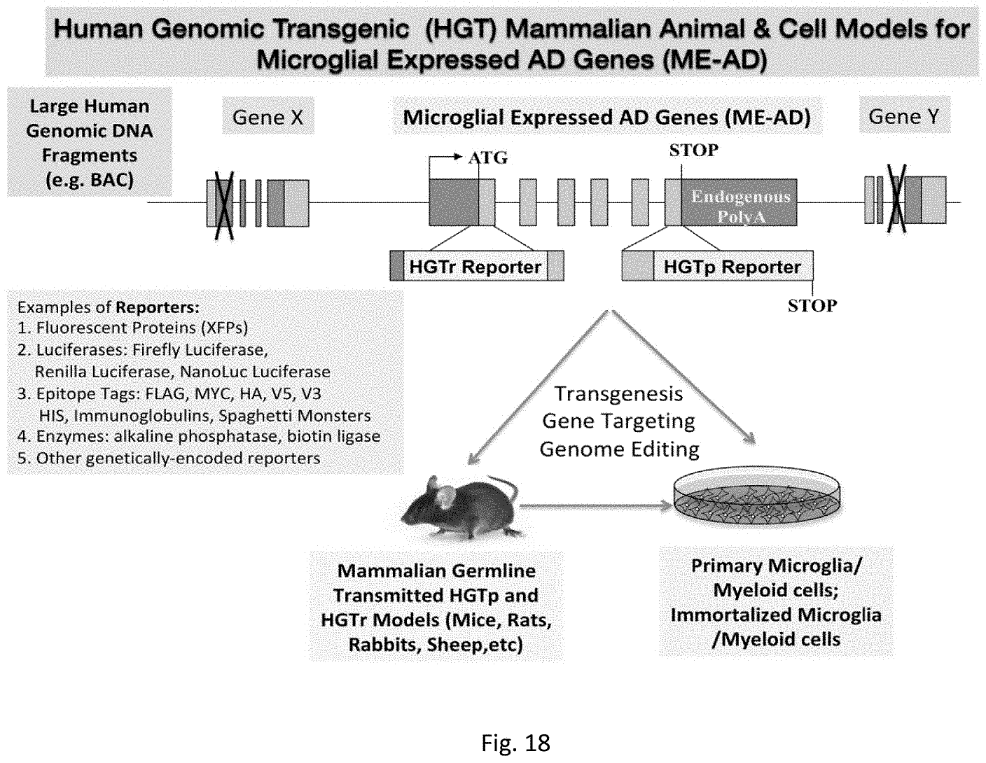

This strategy employs reporter models to express ME-AD genes under human genomic regulation using human genomic DNA fragments that are sufficiently large to contain all the key regulatory elements (e.g. a Bacterial Artificial Chromosome or BAC) for the ME-AD genes (FIG. 18). Example 1 demonstrates that a TREM2 BAC can properly drive the expression of the transgene in microglia, and shows proper disease-associated upregulation of TREM2 in AD mouse brains. This is consistent with prior large-scale BAC transgenesis studies showing the vast majority of BAC transgenes, if the BAC is selected properly, can confer endogenous-like transgene expression for genes located centrally on the BAC (Gong et al., 2003, Nature, 425:917-925). Moreover, another study showed an aneuploid mouse strain carrying human chromosome 21 recapitulated human-specific transcription patterns, suggesting the context of the human genomic transgenes is sufficient to drive human-like gene expression in mice (Wilson et al., 2008). Thus, a properly chosen large human genomic construct, such as a BAC, is sufficient to drive accurate expression of ME-AD genes in relevant mammalian cell types and in genetically-engineered animal models.

The human genomic DNA fragment (e.g. BAC propagated in the bacteria) is engineered using homologous recombination to insert specific reporters into the specific genomic DNA location on the DNA fragment (e.g. Yang et al., 1997, Nat. Biotechnol., 15:859-865; Gong et al., 2002, Genome Res., 12:1992-1998).

At least one essential coding exons of other genes on a given human genomic DNA construct, other than the ME-AD gene of interest, are deleted, to avoid potential confounding factors of overexpression of other genes (non ME-AD genes of interest) on the BAC. Homologous recombination is performed in the bacteria (e.g. Yang et al., 1997, Nat. Biotechnol., 15:859-865; Gong et al., 2002, Genome Res., 12:1992-1998; Warming et al., 2005, Nucleic Acids Res, 33:e36) to delete key exons for all the other genes on the BAC.

The engineered BAC described in Example 1, that had deleted all the other TREM-like genes on the TREM2 BAC, showed selective expression of TREM2 in the microglia at the baseline and proper disease-associated upregulation in AD mouse brains. Therefore, the engineered BAC was further modified for the purposes of expression from a human genomic DNA transgene.

The ME-AD human genomic transgene is inserted into the germline of a mammalian organism through pronuclear injections to develop stably propagated transgenic animal lines (Gong and Yang, 2005, Curr. Protoc. Neurosci., Chapter 5). The random insertion of one or multiple copies of HGT DNA (e.g. BAC) into the genome has been shown to generally not alter the ability of the transgene to confer proper, endogenous-like expression pattern (Gong et al., 2003, Nature, 425:917-925; Lee et al., 2018, Neuron, 97:1032-1048). Alternatively, the transgene is inserted as a single copy into a safe-harbor locus in the genome (Heaney et al., 2004), or is inserted into the endogenous genomic region of the homologous genes through gene targeting (Wallace et al., 2007, Cell, 128, 197-209) or CRISPR/Cas mediated knockin (Yoshimi et al., 2016, Nat Commun, 7:10431).

The human genomic DNA transgene approach is broadly applicable to generate germline HGT reporter mammals in any species that has been used for developing large DNA transgenic animals (e.g. mice, rats, rabbits, sheep, cows).

Cell models are developed by culturing primary microglia or by immortalization of the microglia into microglial-like cell lines that carry the human genomic ME-AD reporter transgene. The microglial immortalization protocols have been established with the expression of oncogenes (e.g. v-myc, SV40) and/or telomerase (e.g. Blasi et al., 1990; Olson et al., 2003; Garcia-Mesa et al., 2017).

The large genomic DNA transgene is used to generate transgenic microglial and myeloid cell lines.

Insertion of Reporters into the Endogenous ME-AD Loci in Human Cell Models (Human Genomic Knock-In or HGKI Models) Using Gene-Targeting or Gene Editing

There is increasing availability of human cell models, including induced pluripotent stem cells or myeloid cells, which can be differentiated into microglial-like or myeloid-like cells (Ryan et al., 2017, Sci Transl Med, 9(421); Pocock and Piers, 2018, Nat Rev Neurosci, 19:445-452). Moreover, there are also transformed human cell lines that exhibit molecular and phenotypic features of microglia or other myeloid cells, and express ME-AD genes. Finally, there are other human cell lines, which may or may not be myeloid-like, but express substantial levels of certain ME-AD genes. Together, these cell models can be used to develop ME-AD reporter cell lines (see more details below) via a gene targeting approach at the endogenous ME-AD genomic locus. The tools for gene targeting can be traditional homologous recombination or the Crispr/Cas-mediated genome editing (i.e. homology-directed repair).

One specific approach uses the human myeloid cell line THP-1, which shows relatively high-level expression of TREM2 as well as its signaling partner DAP12 (TYROBP), to develop human genomic reporter lines for TREM2 (see proteinatlas.org/ENSG00000095970-TREM2/cell; and proteinatlas.org/ENSG00000011600-TYROBP/cell).

Another specific approach uses myeloid cell lines such as HEL, HL-60, HMC-1, NB-4, THP-1, and U-937, to develop human genomic reporter lines for CD33.

Development of ME-AD Protein Reporters in Mammalian Cell and Animal Models Using Human Genomic DNA Constructs

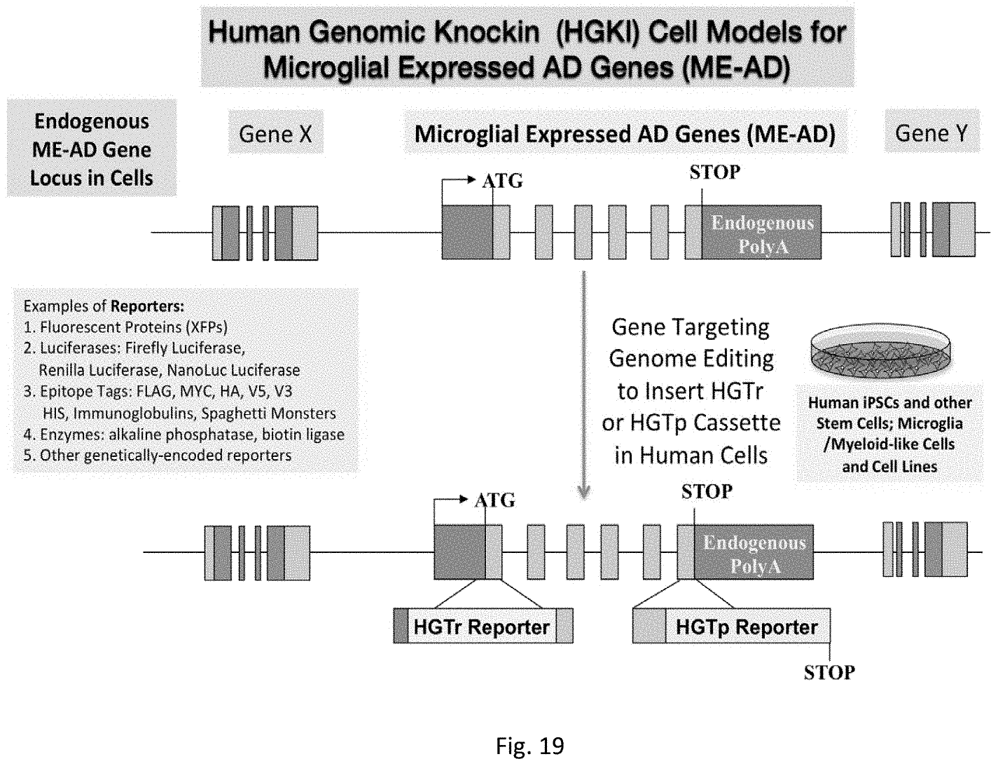

The coding sequence of a Reporter gene was genetically fused with the N- or C-terminal coding DNA sequence of an ME-AD gene on one of the human genomic DNA constructs (i.e. HGT or HGKI; see FIG. 18 and FIG. 19).

A Reporter is defined as protein sequence that can be attached to a cognate protein (e.g. ME-AD protein) and then can be easily detected through direct fluorescence (e.g. XFPs, Giepmans et al., 2006), bioluminescence (e.g. various luciferase such as firefly luciferase and nanoLuc; England et al., 2016, Bioconjug Chem, 27:1175-1187), epitope tags (e.g. FLAG, Myc, His, V5, HA, immunoglobulin tags), or any other protein tags that enables quantitative measurements (e.g. enzymatic reactions such as alkaline phosphatase).

The open reading frame of the Reporter gene can be used in-frame with any of the amino acid near or at the N- or C-terminal coding sequence of the ME-AD gene on the HGT or HGKI genomic DNA. The fusion can be seamless or can be linked by one or more amino acid linker sequence. Moreover, the fusion can result in the loss of one or more amino acid on the ME-AD coding exons on the HGT or HGKI genomic DNA.

The resulting constructs are referred to as HGTp/HGKIp dependent on the genomic construct used. Moreover, the specific reporters can also be denoted, and its relative location of the Reporter to the ME-AD protein can be denoted as “pn” or “pc”. For example, a C-terminal GFP fusion protein on a TREM2 BAC (Example 1), is denoted as “HGTpc-TREM2-GFP.”

For HGTp mammalian animal models, established genetic engineering tools such as homologous recombination based BAC modification (Yang et al., 1997, Nat. Biotechnol., 15:859-865; Gong et al., 2002, Genome Res., 12:1992-1998) are used to precisely engineer the Reporters into an HGT DNA construct propagated in cells. Furthermore, pronuclear injections into fertilized embryos (Gong and Yang, 2005, Curr. Protoc. Neurosci., Chapter 5), gene targeting in embryonic stem cells (Wallace et al., 2007, Cell, 128, 197-209) or CRISPR/Cas mediated knock-in in zygotes (Yoshimi et al., 2016, Nat Commun, 7:10431) are used to develop germline transmitted mammalian models carrying the HGTp reporters either at endogenous or random genomic loci. The proper expression of the HGTp reporters in microglia (as well as other cell types) can be determined by established methods to examine the expression of Reporter RNA (in situ hybridization), protein (immunostaining), or if appropriate, its enzymatic activities. Moreover, the responsivity of the HGTp reporter animal models to disease state (e.g. crossing with other models of disease such as AD) or environmental perturbations (e.g. injury, small molecules) can also be readily evaluated.

HGTp Mammalian Cell Models

HGTp mammalian cell models are developed by immortalization of the microglia or myeloid cells derived from the HGTp animal models; or direct use of the HGTp DNA construct to transfect into microglial or myeloid cell lines or stem cells that could differentiate into microglial or myeloid cell-like cells, and selection of the transfected cells for stable integration of the HGTp constructs. The proper expression of reporters at baseline and in response to perturbation (chemicals, injuries) in the HGTp mammalian cell models can be assessed using RNA, protein, or enzymatic assays as listed above.

HGTpc-TREM2-GFP Transgenic Mice (Also Known as BAC-TREM2-GFP Mice)

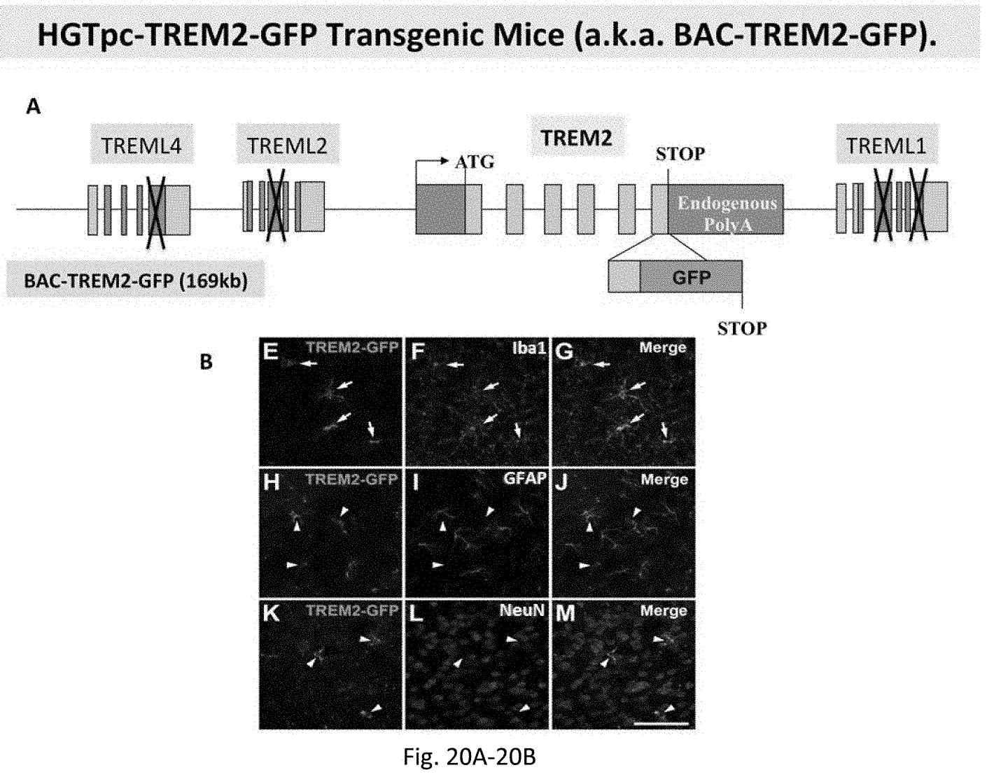

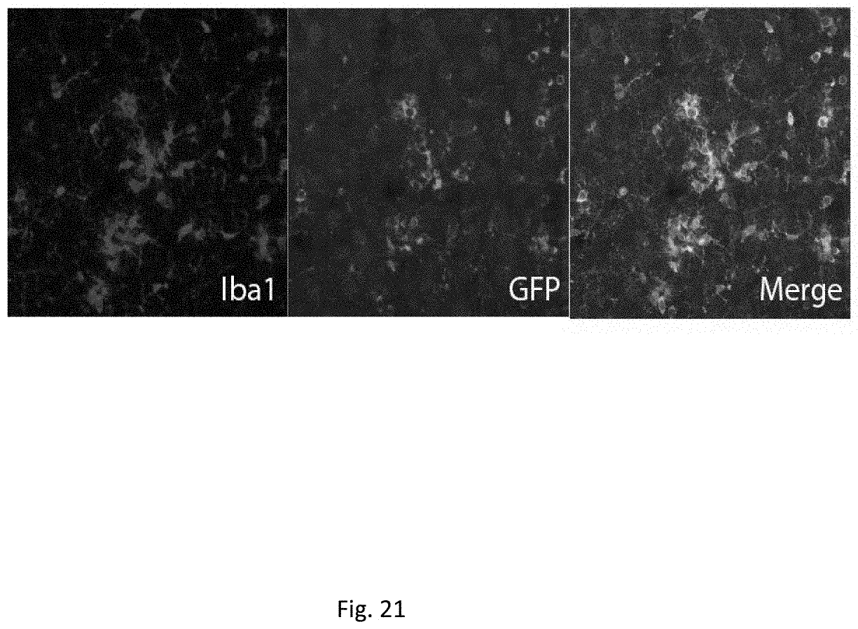

Example 1 demonstrates that a modified human TREM2 BAC with an in frame GFP was used to generate BAC-TREM2-GFP transgenic mice, which showed selective expression of GFP reporter protein (fused with TREM2) only in a subset of microglia at the baseline (about 5.8% in the cortex and 8.7% in the hippocampus), but not in the neurons or astrocytes (FIG. 20). Moreover, double transgenic mice carrying the 5×FAD and BAC-TREM2-GFP transgene have been generated. Importantly, in the reactive Iba1+ microglia in this amyloid model of AD, there is a proper upregulation of the GFP signals in the plaque-associated microglia in the 5×FAD/BAC-TREM2-GFP mice (FIG. 21). This result, and the finding that the human TREM2 transcripts are upregulated in the mid- and late disease stages, show the proper expression of the TREM2 or TREM2-GFP reporters from the BAC transgenic models. In the BAC-TREM2-GFP model, an antibody against GFP can be used to detect the level of TREM2-GFP proteins expressed from the human genomic transgene either in the mouse brain, or in microglia or microglial cell line derived from the BAC-TREM2-GFP mice. Such tools can be used to test molecular therapeutics that can modify the TREM2 protein levels in cells or in vivo.

HGTpc-TREM2-NLuc Transgenic Mice

TREM2 BAC was engineered to insert, in frame and right after the final coding amino acid of TREM2 on the BAC, the coding sequence for nanoLuc (NLuc). NLuc is a modified small luciferase subunit from deep sea shrimp (Oplophorus gracilirostris) and optimized and sold by Promega (Hall et al., 2012, ACS Chem Biol, 7:1848-1857). Compared to other bioluminescent system, NLuc has the advantage of being very small in size (171 amino acid, 19 kDa), having high sensitivity due to >150 fold increase in luminescence (when using the optimal substrate Furimazine), and having high physical stability (Hall et al., 2012, ACS Chem Biol, 7:1848-1857; England et al., 2016, Bioconjug Chem, 27:1175-1187). The BAC-TREM2-NLuc construct was injected into fertilized mouse embryos to generate BAC transgenic mouse lines expressing TREM2-NLuc fusion proteins from the human TREM2 genomic transgenes. These mouse lines are called HGTpc-TREM2-NLuc (or BAC-TREM2-NLuc).

Using the HGTpc-TREM2-NLuc mouse models, primary microglia or generate immortalized microglial cell lines are obtained as cell-based model systems to identify molecular therapeutics. The system can be adapted for high-throughput screening format (e.g. 96 well, 384 well) and use NLuc luminescence assay by adding Furimazine (i.e. Nano-Glo from Promega; England et al., 2016, Bioconjug Chem, 27:1175-1187; Hall et al., 2012, ACS Chem Biol, 7:1848-1857).

Although Furimazine cannot directly across the blood brain barrier (BBB), the HGTpc-TREM2-NLuc facilitates the test of molecular therapeutics that modify the TREM2-NLuc levels in the mammalian brain context. Organotypic brain slices are obtained from the HGTpc-TREM2-NLuc mice and the ability of bioluminescence of the activities is tested ex vivo after Furimazine addition. Alternatively, NLuc activities can be imaged in vivo, as NLuc can be used for imaging in living animals and is shown to provide more sustained luminescence than some other luciferase, e.g. Faussia luciferase (Stacer et al., 2013, Mol Imaging, 12:1-13). The NLuc substrate is injected into the retroorbital space (Birkner et al., 2014, Proc SPIE Int Soc Opt Eng. 8928: 89282F; Caine et al., 2017, J Virol, 91:e01759-16) or delivered into the brain through a cannula (Berglund et al., 2016, Proc Natl Acad Sci USA, 113:E358-67). The detection of luminescence from living animals is then performed using established methods (Mezzanotte et al., 2017, Trends Biotechnol. 35:640-652).

Development of ME-AD RNA Reporters in Mammalian Cell and Animal Models Using Human Genomic DNA Constructs

A Reporter gene is inserted into an exon of the ME-AD gene on a human genomic DNA construct (i.e. HGT or HGKI). The construct enables the expression of the Reporter gene controlled by the genomic transcriptional regulatory elements on the HGT or HGKI construct, and when possible, by the RNA regulatory elements for the cognate ME-AD gene on the genomic construct, however, the ME-AD protein (or the majority of its protein fragments) are not expressed at all. This type of ME-AD messenger RNA reporter construct is referred to as HGTr or HGKIr. The Reporter can be any fluorescent proteins (e.g. XFPs, Giepmans et al., 2006, Science, 312:217-24), bioluminescent enzymes (e.g. luciferase such as firefly luciferase and nanoLuc; England et al., 2016, Bioconjug Chem, 27:1175-1187) immune-epitope tags (e.g. FLAG, Myc, His, V5, HA, immunoglobulin tags), or any other protein-based tags that enables quantitative measurements (e.g. enzymatic reactions such as alkaline phosphatase).

The most common design for such a construct is to insert the Reporter into a 5′ untranslated exon of ME-AD gene, ideally in the exon 1 of ME-AD gene on the genomic construct without a polyadenylation signal (FIG. 18 and FIG. 19), or in a 5′ UTR containing exon other than exon 1 of ME-AD gene on the genomic construct but include an exogenous polyadenylation signal (Gong and Yang, 2005, Curr. Protoc. Neurosci., Chapter 5). The former construct has the advantage that the splicing and 3′ UTR regulatory region of the ME-AD RNA are preserved in the transcript while only the Reporter protein is translated. Hence, it can be considered as a ME-AD RNA reporter that capture all the RNA metabolic events from transcription, splicing, transport, to degradation. The second construct with Reporter inserted into exons other than Exon1 often require the addition of an exogenous polyA signal (Gong and Yang, 2005, Curr. Protoc. Neurosci., Chapter 5), hence such a reporter construct can be considered to report mostly the transcription of the ME-AD RNA, but not other RNA regulatory signals such as splicing, transport, and degradation.

For HGTr mammalian animal models, established genetic engineering tools such as homologous recombination-based BAC modification (Yang et al., 1997, Nat. Biotechnol., 15:859-865; Gong et al., 2002, Genome Res., 12:1992-1998) are used to precisely engineer the Reporters into an HGT DNA construct propagated in cells. Furthermore, pronuclear injections into fertilized embryos (Gong and Yang, 2005, Curr. Protoc. Neurosci., Chapter 5), gene targeting in embryonic stem cells (Wallace et al., 2007, Cell, 128, 197-209) or CRISPR/Cas mediated knock-in in zygotes (Yoshimi et al., 2016, Nat Commun, 7:10431) are used to develop germline transmitted mammalian models carrying the HGTp reporters either at endogenous or random genomic loci. The proper expression of the HGTr reporters in microglia (as well as other cell types) can be determined by established methods to examine the expression of Reporter RNA (in situ hybridization), protein (immunostaining), or if appropriate, its enzymatic activities. Moreover, the responsivity of the HGTr reporter animal models to disease state (e.g. crossing with other models of disease such as AD) or environmental perturbations (e.g. injury, small molecules) can also be readily evaluated.

HGTr Mammalian Cell Models

HGTr mammalian cell models are developed by immortalization of the microglia or myeloid cells derived from the HGTr animal models; or direct use of the HGTr DNA construct to transfect into microglial or myeloid cell lines or stem cells that could differentiate into microglial or myeloid cell-like cells, and selection of the transfected cells for stable integration of the HGTr constructs. The proper expression of reporters at baseline and in response to perturbation (chemicals, injuries) in the HGTr mammalian cell models can be assessed using RNA, protein, or enzymatic assays as listed above.

HGTr-TREM2-NLuc Transgenic Mice.

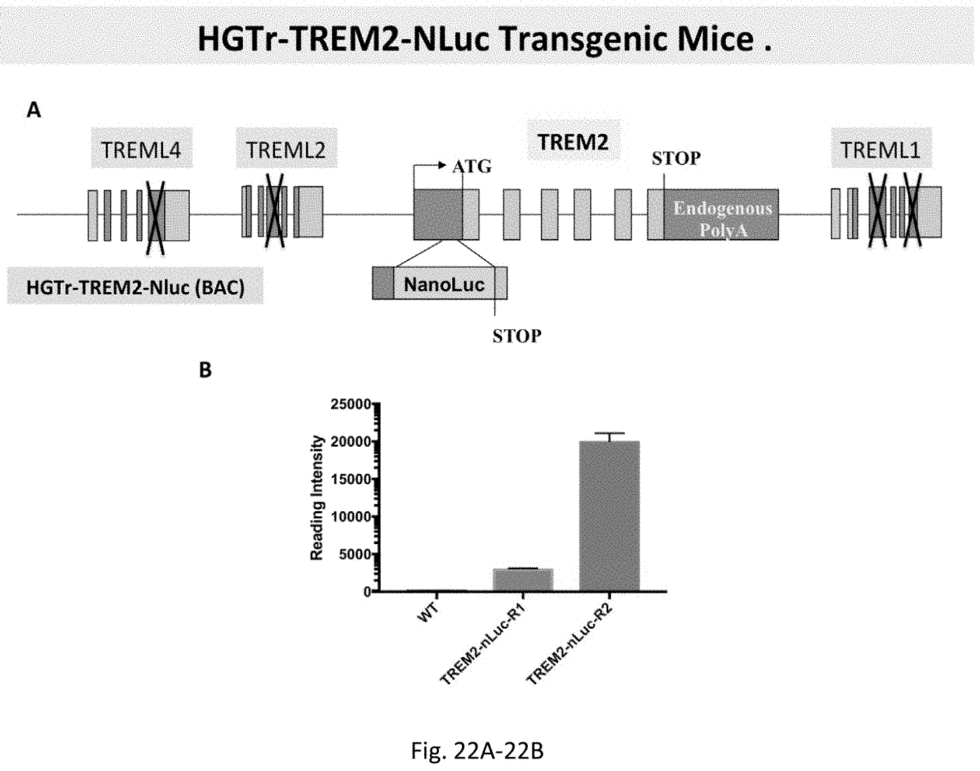

The modified human TREM2 BAC was further modified to insert the coding sequence for NLuc into exon1 of TREM2 on the BAC. The BAC-TREM2-NLuc construct was then injected into fertilized mouse embryos to generate BAC transgenic mouse lines expressing NLuc reporter protein from the human TREM2 genomic transgenes. The genomic construct will express NLuc under the transcriptional regulatory elements of TREM2 on the BAC, and it also preserves all the splicing regions as well as 3′ UTR of TREM2, hence can report other RNA regulatory mechanisms for TREM2 (e.g. splicing, transport, stability). These mouse lines are called HGTr-TREM2-NLuc (or BAC-TREM2r-NLuc). Importantly, brain extracts derived from the brain of two transgenic mouse lines exhibit high NLuc activities at P5 (FIG. 22, the age that TREM2 is expected to be expressed at high levels in the brain (Chertoff et al., 2013, PLoS One, 8:e72083).

With the HGTr-TREM2-NLuc mouse models, primary microglia or generate immortalized microglial cell lines can be obtained as a cell-based model system to identify molecular therapeutics. The system can be adapted for high-throughput screening format (e.g. 96 well, 384 well) and use NLuc luminescence assay by adding Furimazine, as described in detail above.

High-Throughput Screening of Molecules that Can Modulate the Levels of ME-AD RNA or Protein Ex Vivo Using HGTp, HGKIp, HGTr and HGKIr Cell Models.

Cell models carrying human ME-AD genomic Reporters are used to screen for molecules that can modulate the protein or RNA levels of ME-AD genes expressed from the human genomic reporters. Primary microglia or myeloid cells purified from mammalian HGTp and HGTr animal models are plated at appropriate densities onto plates or microplates (e.g. 96 wells, 384 wells, or 1536 wells). Candidate molecular reagents to be tested are added into each well and the cells are incubated. The level of reporters expressed in each well, which indicates ME-AD RNA or protein levels, is assayed with established Reporter-specific methods (e.g. fluorescent signals or bioluminescent signals).

One example of this approach uses primary microglia derived from three BAC transgenic mouse models: HGTpc-TREM2-GFP, HGTpc-TREM2-NLuc, and HFTr-TREM2-NLuc for high-throughput screening. The detection of GFP is done with anti-GFP antibody. The detection of NLuc is done with luminescence upon addition of NLuc substrate, Furimizine.

In another example, immortalized microglia or myeloid cell lines from HGTp and HGTr mammalian animal models are used. The methods for immortalization of microglia or myeloid cells have been published by others, e.g. with the expression of oncogenes (e.g. v-myc, SV40) and/or telomerase (e.g. Blasi et al., 1990; Olson et al., 2003; Garcia-Mesa et al., 2017). Immortalized cell lines are first be assayed for the proper expression of HGTp or HGTr Reporters, and other microglia/myeloid cell specific markers. Subsequently, the established cell lines are plated onto microplates (e.g. 96 wells, 384 wells, or 1536 wells). Candidate molecular reagents to be tested are added into each well and incubated for a specific period of time (e.g. 24 hours.). The level of reporters expressed in each well, which indicates ME-AD RNA or protein levels, is then assayed with established Reporter-specific methods (e.g. fluorescent signals or bioluminescent signals).

In yet another example, the HGKIp and HGKIr cell lines as described elsewhere herein are used. The human cells used to develop the HGKI reporters are immortalized microglial or myeloid cell lines (e.g. THP-1), or microglia-like cells differentiated from iPSCs or myeloid cell (Ryan et al., 2017; Pocok and Piers, 2018). The Reporters are inserted by gene targeting or CRISPR/Cas-dependent homology-directed repair to the endogenous ME-AD loci in these cells. Next the HGKIp or HGKIr cells are characterized for the proper expression of Reporters, and other microglia/myeloid cell specific markers. Subsequently, the established cell lines can be plated onto microplates (e.g. 96 wells, 384 wells, or 1536 wells). Candidate molecular reagents to be tested can be added into each well and incubate for a period of time. The level of reporters expressed in each well, which indicates ME-AD RNA or protein levels, are then assayed with established Reporter-specific methods (e.g. fluorescent signals or bioluminescent signals).

The Molecules can be added to the aforementioned ME-AD cell models in a low-, medium- or high-throughput screening. Molecules are defined as small molecules (e.g. known target libraries, chemically-diverse small molecule libraries), natural compounds, large molecules (antibodies, aptmers), nucleic acid related molecules (RNA, DNA, oligonucleotides), or other complex molecules.

Scalable Testing of Molecules that Can Modulate the Levels of ME-AD RNA or Protein In Vivo Using HGTp and HGTr Mammalian Animal Models.

The HGTp and HGTr in vivo mammalian animal models are used to test the ability of Molecules to modulate the expression of ME-AD RNA or protein that are expressed from these reporters either in the brain (e.g. microglia) or in the periphery cells (e.g. myeloid cells).

The assay of the Reporters in HGTp or HGTr can be performed in intact tissues (brain, periphery) when the Reporters uses an enzymatic activity (e.g. luciferase) or emits fluorescent signals (e.g. XFPs) that can be quantitatively assayed in intact animals. For example, HGTp and HGTr that express firefly luciferase (fLuc) or NLuc in a transgenic animal. For fLuc, D-luciferin is injected intraperitonially into the HGTP or HGTr reporter mice, and the luciferase reporter activities (bioluminescence) are obtained using a CCD camera. For NLuc, the substrate Fumirazine is either delivered into the appropriate brain regions via a cannula or by retroorbital injections 10-15 minutes prior to imaging with CCD camera. Molecules to be tested for modulation of ME-AD Reporter expression can be delivered PO, IP or IV if the molecule is known to cross BBB, or alternatively it can be delivered into the CNS space via direct cannula infusion, retroorbital injections, or injection into the intrathecal space.

In another example, one or more test molecules are injected into HGTp or HGTr reporter animals via one of the injection routs mentioned above (IP, IV, PO, retroorbital or direct delivery into the CNS space, see above), and subsequently after a period of incubation time, the brains are dissected and the Reporter activity is assayed either in the whole brain tissue, in tissues from a specific brain region, or in microglia or myeloid cells purified from a specific tissue.

The disclosures of each and every patent, patent application, and publication cited herein are hereby incorporated herein by reference in their entirety. While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a microglial or myeloid expressed Alzheimer's disease associated (ME-AD) gene reporter construct comprising at least one selected from the group consisting of:
a) a genomic regulatory element of a ME-AD gene operably linked to at least one sequence encoding a reporter molecule; and
b) a ME-AD gene operably linked to at least one sequence encoding a reporter molecule.

2. (canceled)

3. The composition of claim 1, wherein the genomic regulatory element is selected from the group consisting of a promoter, a transcriptional enhancer, a transcriptional repressor, a locus control region, a splicing regulatory element, a mRNA polyadenylation site, a trafficking element and a stability regulatory element.

4. The composition of claim 1, comprising a ME-AD gene operably linked to at least one sequence encoding a reporter molecule.

5. The composition of claim 1, wherein the ME-AD gene is selected from the group consisting of TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN and MEF2C.

6. The composition of claim 5, wherein the ME-AD gene is TREM2.

7. The composition of claim 1, wherein the reporter molecule is luciferase.

8. The composition of claim 1, wherein the reporter construct is a bacterial artificial chromosome (BAC).

9. The composition of claim 1, wherein the reporter construct is integrated into the genome of a cell.

10. A cell comprising a ME-AD reporter construct of claim 1.

11. A germline-transmitted genome engineered animal comprising a ME-AD reporter construct of claim 1.

12. A method of screening for a modulator of a ME-AD gene, the method comprising:
a) contacting a cell comprising at least one ME-AD reporter construct with an agent,
b) measuring the expression level of at least one reporter molecule, and
c) comparing the expression level of at least one reporter molecule to the level of a comparator control.

13. The method of claim 12, wherein the ME-AD reporter construct comprises at least one selected from the group consisting of a ME-AD gene and a genomic regulatory element of a ME-AD gene operably linked to at least one sequence encoding a reporter molecule.

14. The method of claim 13, wherein the genomic regulatory element is selected from the group consisting of a promoter, a transcriptional enhancer, a transcriptional repressor, a locus control region, a splicing regulatory element, a mRNA polyadenylation site, a trafficking element and a stability regulatory element.

15. The method of claim 12, wherein the ME-AD gene is selected from the group consisting of TREM2, DAP12 (TYROBP), APOE, CD33, PLCG2, SPI1, ABI3, ABCA7, PTK2B, RIN3, SORL1, ZCWPW1, CR1, NME8, BIN1, MS4A4A, MS4A6A, MS4A4E, MS4A6E, MS4A2, IL1RAP, INPP5D, PICALM, HLA-DRB1, CASS4, CD2AP, EPHA1, GRN and MEF2C.

16. The method of claim 15, wherein the ME-AD gene is TREM2.

17. The method of claim 12, wherein the reporter molecule is luciferase.

18. The method of claim 12, wherein the reporter construct is integrated into the genome of a cell line.

19. The method of claim 12, wherein the reporter construct is on a BAC.

20. The method of claim 12, wherein the comparator control is at least one selected from the group consisting of: a positive control, a negative control, a historical control, a historical norm, and the level of a reference molecule in the biological sample.

21. The method of claim 12, wherein the agent is selected from the group consisting of a small interfering RNA (siRNA), a small guide RNA (gRNA), a microRNA, an antisense or sense nucleic acid, a ribozyme, an expression vector encoding a transdominant negative mutant, an antibody, a peptide, a chemical compound and a small molecule.
